(19) United States
(12) Patent Application Publication
(10) Pub. No.: US 2025/0393466 A1
(43) Pub. Date: Dec. 25, 2025

(54) LIGHT-EMITTING DEVICE, LIGHT-EMITTING APPARATUS, LIGHT-EMITTING MODULE, ELECTRONIC APPARATUS, LIGHTING APPARATUS, ORGANOMETALLIC COMPLEX, LIGHT-EMITTING MATERIAL, ORGANIC COMPOUND, AND DINUCLEAR COMPLEX

(71) Applicant: Semiconductor Energy Laboratory Co., Ltd., Atsugi-shi (JP)

(72) Inventors: Satoshi SEO, Sagamihara (JP); Tomoya YAMAGUCHI, Atsugi (JP); Hideko YOSHIZUMI, Atsugi (JP); Takeyoshi WATABE, Atsugi (JP)

(21) Appl. No.: 19/307,231

(22) Filed: Aug. 22, 2025

Related U.S. Application Data
(63) Continuation of application No. 17/264,456, filed on Jan. 29, 2021, now abandoned.

(30) Foreign Application Priority Data
Sep. 27, 2018 (JP) 2018-182792
Jan. 30, 2019 (JP) 2019-014156

(51) Int. Cl. H10K 85/30

(52) U.S. Cl. CPC H10K 85/342; H10K 85/361

(57) ABSTRACT

The emission efficiency of a light-emitting device that emits near-infrared light is increased. The reliability of a light-emitting device that emits near-infrared light is increased. A light-emitting device using an organic compound that emits light having a maximum peak wavelength greater than or equal to 760 nm and less than or equal to 900 nm is provided. An organometallic complex represented by General Formula (G1) is provided. In the formula, each of R1 to R11 independently represents hydrogen or an alkyl group having 1 to 6 carbon atoms; at least two of R1 to R4 and at least two of R5 to R9 represent an alkyl group having 1 to 6 carbon atoms; X represents a substituted or unsubstituted benzene ring or naphthalene ring; n is 2 or 3; and L represents a monoanionic ligand.

### TECHNICAL FIELD

One embodiment of the present invention relates to a light-emitting device, a light-emitting apparatus, a light-emitting module, an electronic apparatus, a lighting apparatus, an organometallic complex, a light-emitting material, an organic compound, and a dinuclear complex. One embodiment of the present invention relates to a light-emitting device, a light-emitting apparatus, a light-emitting module, an electronic apparatus, a lighting apparatus, an organometallic complex, a light-emitting material, an organic compound, and a dinuclear complex, each of which emits near-infrared light.

Note that one embodiment of the present invention is not limited to the above technical field. Examples of the technical field of one embodiment of the present invention include a semiconductor apparatus, a display apparatus, a light-emitting apparatus, a power storage apparatus, a memory apparatus, an electronic apparatus, a lighting apparatus, an input apparatus (e.g., a touch sensor), an input/output apparatus (e.g., a touch panel), a driving method thereof, and a manufacturing method thereof.

### BACKGROUND ART

Research and development have been actively conducted on light-emitting devices using organic electroluminescence (EL) phenomenon (also referred to as organic EL devices or organic EL elements). In a basic structure of an organic EL device, a layer containing a light-emitting organic compound (hereinafter also referred to as a light-emitting layer) is sandwiched between a pair of electrodes. By application of voltage to the organic EL device, light emitted from the light-emitting organic compound can be obtained.

An example of the light-emitting organic compound is a compound capable of converting a triplet excited state into light (also referred to as a phosphorescent compound or a phosphorescent material). As a phosphorescent material, Patent Document 1 discloses an organometallic complex that contains iridium or the like as a central metal.

Image sensors have been used in a variety of applications such as personal authentication, defect analysis, medical diagnosis, and security. The wavelength of light sources used for image sensors is different depending on applications. Light having a variety of wavelengths, for example, light having a short wavelength, such as visible light and X-rays, and light having a long wavelength, such as near-infrared light, is used for image sensors.

Light-emitting devices have been considered to be applied to light sources of image sensors such as the above in addition to display apparatuses.

### REFERENCE

### Patent Document

[Patent Document 1] Japanese Published Patent Application No. 2007-137872

### SUMMARY OF THE INVENTION

### Problems to be Solved by the Invention

An object of one embodiment of the present invention is to increase the emission efficiency of a light-emitting device that emits near-infrared light. An object of one embodiment of the present invention is to increase the reliability of a light-emitting device that emits near-infrared light. An object of one embodiment of the present invention is to increase the lifetime of a light-emitting device that emits near-infrared light.

An object of one embodiment of the present invention is to provide an organometallic complex having high emission efficiency. An object of one embodiment of the present invention is to provide an organometallic complex having high chemical stability. An object of one embodiment of the present invention is to provide a novel organometallic complex that emits near-infrared light. An object of one embodiment of the present invention is to provide a novel organometallic complex that can be used in an EL layer of a light-emitting device.

Note that the description of these objects does not preclude the existence of other objects. One embodiment of the present invention does not need to achieve all these objects. Other objects can be derived from the descriptions of the specification, the drawings, and the claims.

### Means for Solving the Problems

One embodiment of the present invention is a light-emitting device (also referred to as a light-emitting element) including a light-emitting layer. The light-emitting layer includes a light-emitting organic compound. The maximum peak wavelength (also referred to as wavelength at which the peak intensity is the highest) of light emitted from the light-emitting organic compound is greater than or equal to 760 nm and less than or equal to 900 nm.

One embodiment of the present invention is a light-emitting device including a first electrode, a second electrode, and a light-emitting layer. The light-emitting layer is positioned between the first electrode and the second electrode. The light-emitting layer includes a light-emitting organic compound. The maximum peak wavelength of light emitted from the light-emitting organic compound is greater than or equal to 760 nm and less than or equal to 900 nm.

The maximum peak wavelength of light emitted from the light-emitting organic compound is preferably greater than or equal to 780 nm. In addition, the maximum peak wavelength of light emitted from the light-emitting organic compound is preferably less than or equal to 880 nm.

The light-emitting organic compound is preferably an organometallic complex having a metal-carbon bond. In particular, the light-emitting organic compound is further preferably a cyclometalated complex. Moreover, the light-emitting organic compound is preferably an orthometalated complex. In addition, the light-emitting organic compound is preferably an iridium complex. When the light-emitting organic compound is an organometallic complex having a metal-carbon bond, it is preferred that the organometallic complex include a condensed heteroaromatic ring including 2 to 5 rings and the condensed heteroaromatic ring be coordinated to a metal. The condensed heteroaromatic ring preferably includes 3 or more rings. Moreover, the condensed heteroaromatic ring preferably includes 4 or less rings.

One embodiment of the present invention is a light-emitting apparatus that includes the light-emitting device having any of the above-described structures, and one or both of a transistor and a substrate.

One embodiment of the present invention is a light-emitting module including the above-described light-emitting apparatus, where a connector such as a flexible printed circuit (hereinafter referred to as FPC) or a TCP (Tape Carrier Package) is attached or an integrated circuit (IC) is mounted by a COG (Chip On Glass) method, a COF (Chip On Film) method, or the like. Note that the light-emitting module of one embodiment of the present invention may include only one of a connector and an IC or may include both of them.

One embodiment of the present invention is an electronic apparatus including the above-described light-emitting module and at least one of an antenna, a battery, a housing, a camera, a speaker, a microphone, and an operation button.

One embodiment of the present invention is a lighting apparatus including the above-described light-emitting apparatus and at least one of a housing, a cover, and a support.

One embodiment of the present invention is an organometallic complex represented by General Formula (G1). Another embodiment of the present invention is a light-emitting material represented by General Formula (G1). Another embodiment of the present invention is a light-emitting device material represented by General Formula (G1).

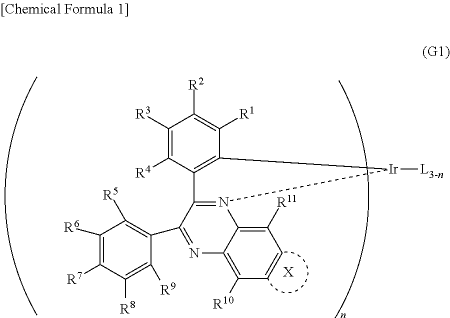

In General Formula (G1), each of R¹ to R¹¹ independently represents hydrogen or an alkyl group having 1 to 6 carbon atoms; at least two of R¹ to R⁴ represent an alkyl group having 1 to 6 carbon atoms; at least two of R⁵ to R⁹ represent an alkyl group having 1 to 6 carbon atoms; X represents a substituted or unsubstituted benzene ring or naphthalene ring; n is 2 or 3; and L represents a monoanionic ligand.

One embodiment of the present invention is an organometallic complex represented by General Formula (G2). Another embodiment of the present invention is a light-emitting material represented by General Formula (G2). Another embodiment of the present invention is a light-emitting device material represented by General Formula (G2).

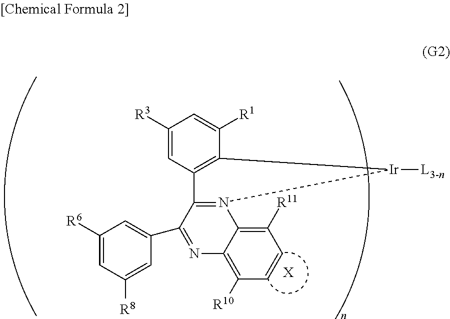

In General Formula (G2), each of R¹, R³, R⁶, and R⁸ independently represents an alkyl group having 1 to 6 carbon atoms; each of R¹⁰ and R¹¹ independently represents hydrogen or an alkyl group having 1 to 6 carbon atoms; X represents a substituted or unsubstituted benzene ring or naphthalene ring; n is 2 or 3; and L represents a monoanionic ligand.

The maximum peak wavelength of light emitted from the organometallic complex, the light-emitting material, or the light-emitting device material of one embodiment of the present invention is preferably greater than or equal to 760 nm and less than or equal to 900 nm.

One embodiment of the present invention is a light-emitting device including a light-emitting layer. The light-emitting layer includes the organometallic complex, the light-emitting material, or the light-emitting device material, each of which has any of the above-described structures. The light-emitting device has a function of emitting light having a maximum peak wavelength greater than or equal to 760 nm and less than or equal to 900 nm.

One embodiment of the present invention is an organic compound represented by General Formula (G0).

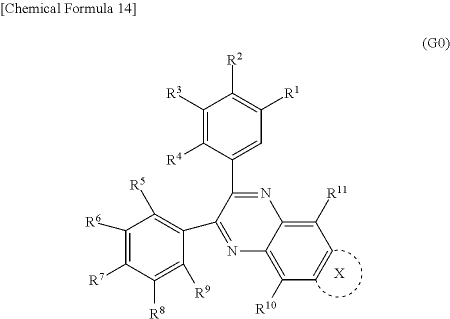

In General Formula (G0), each of R¹ to R¹¹ independently represents hydrogen or an alkyl group having 1 to 6 carbon atoms; at least two of R¹ to R⁴ represent an alkyl group having 1 to 6 carbon atoms; at least two of R⁵ to R⁹ represent an alkyl group having 1 to 6 carbon atoms; and X represents a substituted or unsubstituted benzene ring or naphthalene ring.

One embodiment of the present invention is an organic compound represented by Structural Formula (200).

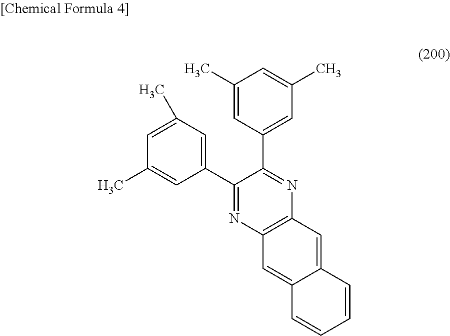 [SMILES: Cc1cc(C)cc(-c2nc3cc4ccccc4cc3nc2-c2cc(C)cc(C)c2)c1]

One embodiment of the present invention is a dinuclear complex represented by General Formula (B).

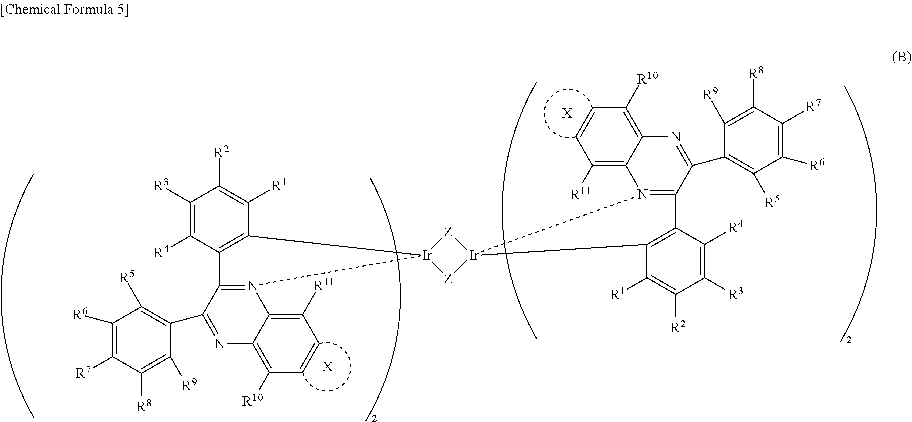

In General Formula (B), Z represents a halogen; each of R¹ to R¹¹ independently represents hydrogen or an alkyl group having 1 to 6 carbon atoms; at least two of R¹ to R⁴ represent an alkyl group having 1 to 6 carbon atoms; at least two of R⁵ to R⁹ represent an alkyl group having 1 to 6 carbon atoms; and X represents a substituted or unsubstituted benzene ring or naphthalene ring.

One embodiment of the present invention is a dinuclear complex represented by Structural Formula (210).

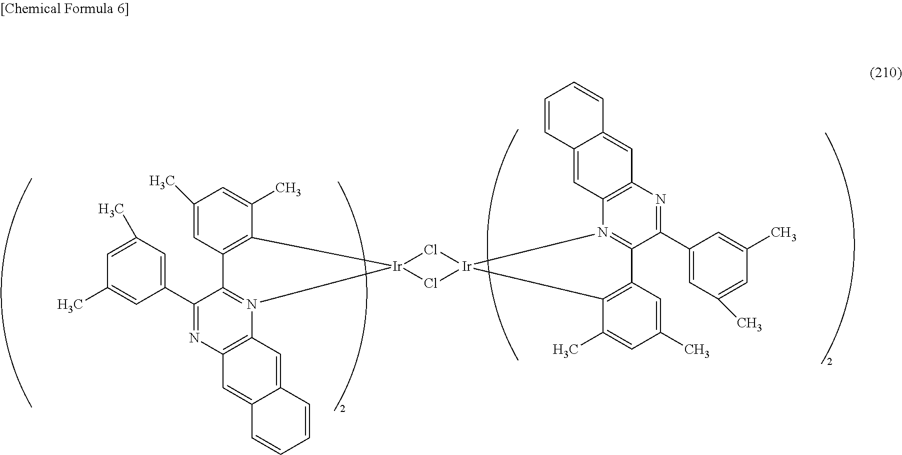

### Effect of the Invention

According to one embodiment of the present invention, the emission efficiency of a light-emitting device that emits near-infrared light can be increased. According to one embodiment of the present invention, the reliability of a light-emitting device that emits near-infrared light can be increased. According to one embodiment of the present invention, the lifetime of a light-emitting device that emits near-infrared light can be increased.

According to one embodiment of the present invention, an organometallic complex having high emission efficiency can be provided. According to one embodiment of the present invention, an organometallic complex having high chemical stability can be provided. According to one embodiment of the present invention, a novel organometallic complex that emits near-infrared light can be provided. According to one embodiment of the present invention, a novel organometallic complex that can be used in an EL layer of a light-emitting device can be provided.

Note that the description of these effects does not preclude the existence of other effects. One embodiment of the present invention does not need to have all these effects. Other effects can be derived from the descriptions of the specification, the drawings, and the claims.

### BRIEF DESCRIPTION OF THE DRAWINGS

FIG. 1A, FIG. 1B, and FIG. 1C are cross-sectional views illustrating examples of light-emitting devices.

FIG. 2A is a top view illustrating an example of a light-emitting apparatus. FIG. 2B and FIG. 2C are cross-sectional views illustrating examples of the light-emitting apparatus.

FIG. 3A is a top view illustrating an example of a light-emitting apparatus. FIG. 3B is a cross-sectional view illustrating an example of the light-emitting apparatus.

FIG. 4A to FIG. 4E are diagrams illustrating examples of electronic apparatuses.

FIG. 5 is a ¹H-NMR chart of the organic compound represented by Structural Formula (200).

FIG. 6 is a ¹H-NMR chart of the organometallic complex represented by Structural Formula (100).

FIG. 7 shows an ultraviolet-visible absorption spectrum and an emission spectrum of the organometallic complex represented by Structural Formula (100).

FIG. 8 is a graph showing the weight change rate of the organometallic complex represented by Structural Formula (100).

FIG. 9A and FIG. 9B are cross-sectional views illustrating light-emitting devices in Examples.

FIG. 10 is a diagram showing the current density-radiant emittance characteristics of a light-emitting device 1.

FIG. 11 is a diagram showing the voltage-current density characteristics of the light-emitting device 1.

FIG. 12 is a diagram showing the current density-radiant flux characteristics of the light-emitting device 1.

FIG. 13 is a diagram showing the voltage-radiant emittance characteristics of the light-emitting device 1.

FIG. 14 is a diagram showing the current density-external quantum efficiency characteristics of the light-emitting device 1.

FIG. 15 is a diagram showing the emission spectrum of the light-emitting device 1.

FIG. 16 is a diagram showing reliability test results of the light-emitting device 1.

FIG. 17 is a diagram showing the current density-radiant emittance characteristics of a light-emitting device 2.

FIG. 18 is a diagram showing the voltage-current density characteristics of the light-emitting device 2.

FIG. 19 is a diagram showing the current density-radiant flux characteristics of the light-emitting device 2.

FIG. 20 is a diagram showing the voltage-radiant emittance characteristics of the light-emitting device 2.

FIG. 21 is a diagram showing the current density-external quantum efficiency characteristics of the light-emitting device 2.

FIG. 22 is a diagram showing the emission spectrum of the light-emitting device 2.

FIG. 23 is a diagram showing reliability test results of the light-emitting device 2.

FIG. 24 is a diagram showing the current density-radiant emittance characteristics of a light-emitting device 3.

FIG. 25 is a diagram showing the voltage-current density characteristics of the light-emitting device 3.

FIG. 26 is a diagram showing the current density-radiant flux characteristics of the light-emitting device 3.

FIG. 27 is a diagram showing the voltage-radiant emittance characteristics of the light-emitting device 3.

FIG. 28 is a diagram showing the current density-external quantum efficiency characteristics of the light-emitting device 3.

FIG. 29 is a diagram showing the emission spectrum of the light-emitting device 3.

FIG. 30 is a diagram showing reliability test results of the light-emitting device 3.

FIG. 31 is a diagram showing angle dependence of the relative intensity of the light-emitting device 3.

FIG. 32 is a diagram showing angle dependence of the normalized photon intensity of the light-emitting device 3.

### MODE FOR CARRYING OUT THE INVENTION

Embodiments will be described in detail with reference to the drawings. Note that the present invention is not limited to the following description, and it will be readily appreciated by those skilled in the art that modes and details of the present invention can be modified in various ways without departing from the spirit and scope of the present invention. Thus, the present invention should not be construed as being limited to the description in the following embodiments.

Note that in the structures of the present invention described below, the same portions or portions having similar functions are denoted by the same reference numerals in different drawings, and description thereof is not repeated. Furthermore, the same hatch pattern is used for portions having similar functions, and the portions are not especially denoted by reference numerals in some cases.

The position, size, range, or the like of each component illustrated in drawings does not represent the actual position, size, range, or the like in some cases for easy understanding. Therefore, the disclosed invention is not necessarily limited to the position, size, range, or the like disclosed in the drawings.

Note that the term “film” and the term “layer” can be interchanged with each other depending on the case or circumstances. For example, the term “conductive layer” can be changed into the term “conductive film”. As another example, the term “insulating film” can be changed into the term “insulating layer”.

### Embodiment 1

In this embodiment, an organometallic complex of one embodiment of the present invention will be described.

### Structure of Organometallic Complex of One Embodiment of the Present Invention

In the organometallic complex of one embodiment of the present invention, a ligand having a benzoquinoxaline skeleton or a naphthoquinoxaline skeleton is coordinated to iridium that is a central metal. Specifically, one embodiment of the present invention is an organometallic complex represented by General Formula (G1). Another embodiment of the present invention is a light-emitting material represented by General Formula (G1). Another embodiment of the present invention is a light-emitting device material represented by General Formula (G1).

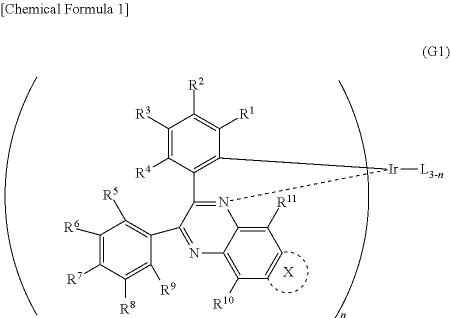

In General Formula (G1), each of R¹ to R¹¹ independently represents hydrogen or an alkyl group having 1 to 6 carbon atoms; at least two of R¹ to R⁴ represent an alkyl group having 1 to 6 carbon atoms; at least two of R⁵ to R⁹ represent an alkyl group having 1 to 6 carbon atoms; X represents a substituted or unsubstituted benzene ring or naphthalene ring; n is 2 or 3; and L represents a monoanionic ligand.

In General Formula (G1), X is a substituted or unsubstituted benzene ring or naphthalene ring, that is, a benzene ring or a naphthalene ring is fused to quinoxaline, whereby a x-conjugated system can be extended, the lowest unoccupied molecular orbital level (LUMO level) can be deepened, and energetic stability is obtained; hence, the emission wavelength can be a long wavelength. Thus, an organometallic complex that emits near-infrared light can be obtained.

One embodiment of the present invention is an organometallic complex represented by General Formula (G2). Another embodiment of the present invention is a light-emitting material represented by General Formula (G2). Another embodiment of the present invention is a light-emitting device material represented by General Formula (G2).

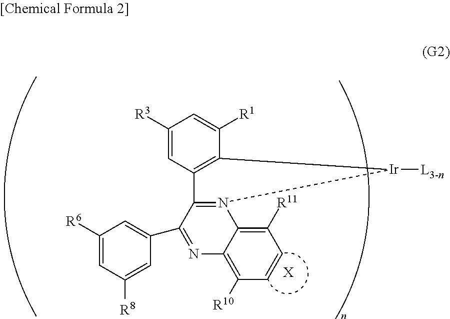

In General Formula (G2), each of R¹, R³, R⁶, and R⁸ independently represents an alkyl group having 1 to 6 carbon atoms; each of R¹⁰ and R¹¹ independently represents hydrogen or an alkyl group having 1 to 6 carbon atoms; X represents a substituted or unsubstituted benzene ring or naphthalene ring; n is 2 or 3; and L represents a monoanionic ligand.

In General Formula (G2), X is a substituted or unsubstituted benzene ring or naphthalene ring, that is, a benzene ring or a naphthalene ring is fused to quinoxaline, whereby a x-conjugated system can be extended, the LUMO level can be deepened, and energetic stability is obtained; hence, the emission wavelength can be a long wavelength. Thus, an organometallic complex that emits near-infrared light can be obtained.

It is preferred that R¹, R³, R⁶, and R⁸ be each an alkyl group having 1 to 6 carbon atoms, in which case the sublimability of the organometallic complex increases and the sublimation temperature can be lowered, as compared to the case where R¹, R³, R⁶, and R⁸ are hydrogen. In particular, each of R¹, R³, R⁶, and R⁸ is preferably a methyl group. That is, all of R¹, R³, R⁶, and R⁸ are preferably methyl groups.

In one embodiment of the present invention, X is a substituted or unsubstituted benzene ring or naphthalene ring; hence, the sublimability of the organometallic complex is likely to be low as compared to the case where X is not a condensed ring. However, since R¹, R³, R⁶, and R⁸ are each an alkyl group having 1 to 6 carbon atoms, the sublimability of the organometallic complex can be increased. Thus, an organometallic complex that has high sublimability and emits near-infrared light can be obtained.

Since R¹ and R³ are each an alkyl group having 1 to 6 carbon atoms, the dihedral angle of the benzene ring bonded to iridium can be increased. Consequently, the secondary peak in the emission spectrum of the organometallic complex can be theoretically reduced, so that the half width can be reduced. Thus, light with a desired wavelength can be obtained efficiently.

Specific examples of the alkyl group having 1 to 6 carbon atoms in General Formula (G1) and General Formula (G2) include a methyl group, an ethyl group, a propyl group, an isopropyl group, a butyl group, a sec-butyl group, an isobutyl group, a tert-butyl group, a pentyl group, an isopentyl group, a sec-pentyl group, a tert-pentyl group, a neopentyl group, a hexyl group, an isohexyl group, a 3-methylpentyl group, a 2-methylpentyl group, a 2-ethylbutyl group, a 1,2-dimethylbutyl group, and a 2,3-dimethylbutyl group.

When the benzene ring or the naphthalene ring has a substituent in General Formula (G1) and General Formula (G2), the substituent can be an alkyl group having 1 to 6 carbon atoms. The above description can be referred to for the alkyl group having 1 to 6 carbon atoms.

Examples of the monoanionic ligand include a monoanionic bidentate chelate ligand having a β-diketone structure, a monoanionic bidentate chelate ligand having a carboxyl group, a monoanionic bidentate chelate ligand having a phenolic hydroxyl group, a monoanionic bidentate chelate ligand in which two ligand elements are both nitrogen, and a bidentate ligand that forms a metal-carbon bond with iridium by cyclometalation.

The monoanionic ligand is preferably any one of General Formulae (L1) to (L7). In particular, the use of the ligand represented by General Formula (L1) is preferable, in which case the sublimability increases. Furthermore, the ligand represented by General Formula (L8) (dipivaloyl methane), which is an example of the ligand represented by General Formula (L1), and the ligand having a benzoquinoxaline skeleton or a naphthoquinoxaline skeleton form a suitable combination, which is preferable because the sublimability of the organometallic complex of one embodiment of the present invention increases and the sublimation temperature can be lowered.

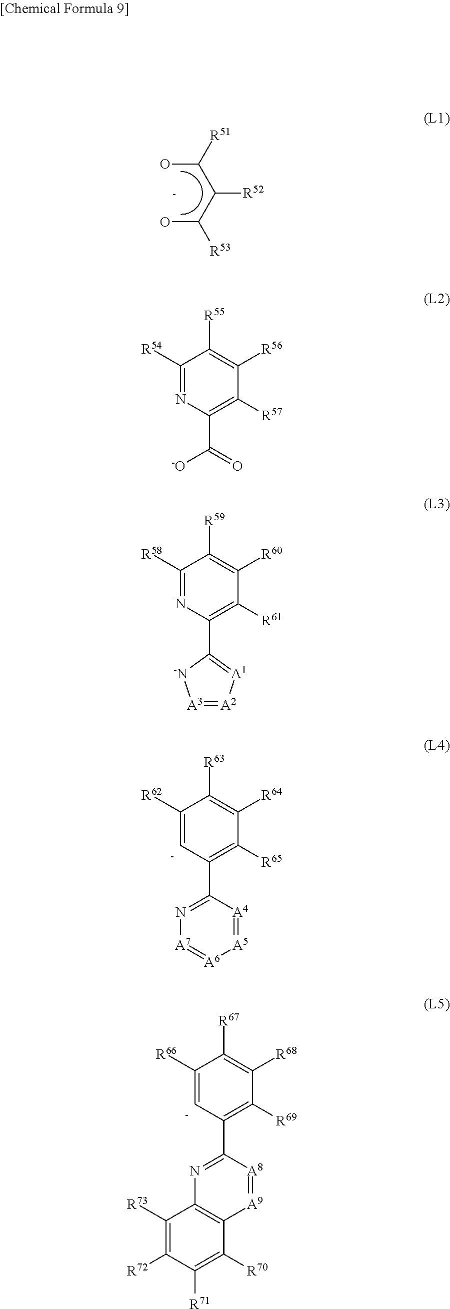

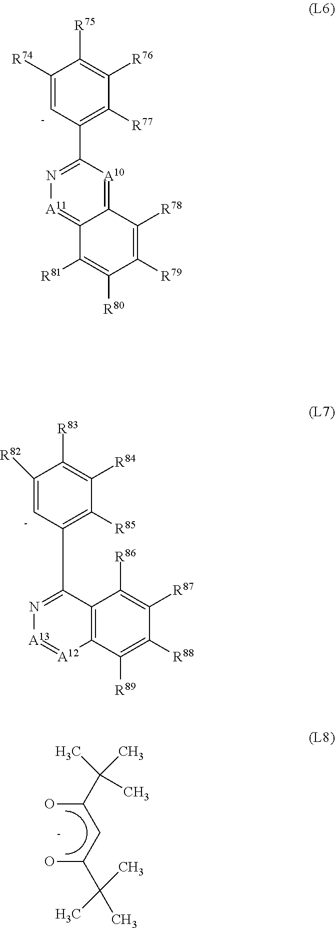

In General Formulae (L1) to (L7), each of R⁵¹ to R⁸⁹ independently represents hydrogen, a substituted or unsubstituted alkyl group having 1 to 6 carbon atoms, a halogeno group, a vinyl group, a substituted or unsubstituted haloalkyl group having 1 to 6 carbon atoms, a substituted or unsubstituted alkoxy group having 1 to 6 carbon atoms, a substituted or unsubstituted alkylthio group having 1 to 6 carbon atoms, or a substituted or unsubstituted aryl group having 6 to 13 carbon atoms; each of A¹ to A¹³ independently represents nitrogen, sp² hybridized carbon bonded to hydrogen, or sp² hybridized carbon having a substituent; and the substituent represents any of an alkyl group having 1 to 6 carbon atoms, a halogeno group, a haloalkyl group having 1 to 6 carbon atoms, and a phenyl group.

The maximum peak wavelength (i.e., the wavelength at which the peak intensity is the highest) of light emitted from the organometallic complex of one embodiment of the present invention is preferably greater than or equal to 760 nm and less than or equal to 900 nm. The wavelength is particularly preferably greater than or equal to 780 nm. Moreover, the wavelength is preferably less than or equal to 880 nm.

Specific examples of the organometallic complex of one embodiment of the present invention include organometallic complexes represented by Structural Formula (100) to Structural Formula (111). Note that the present invention is not limited thereto.

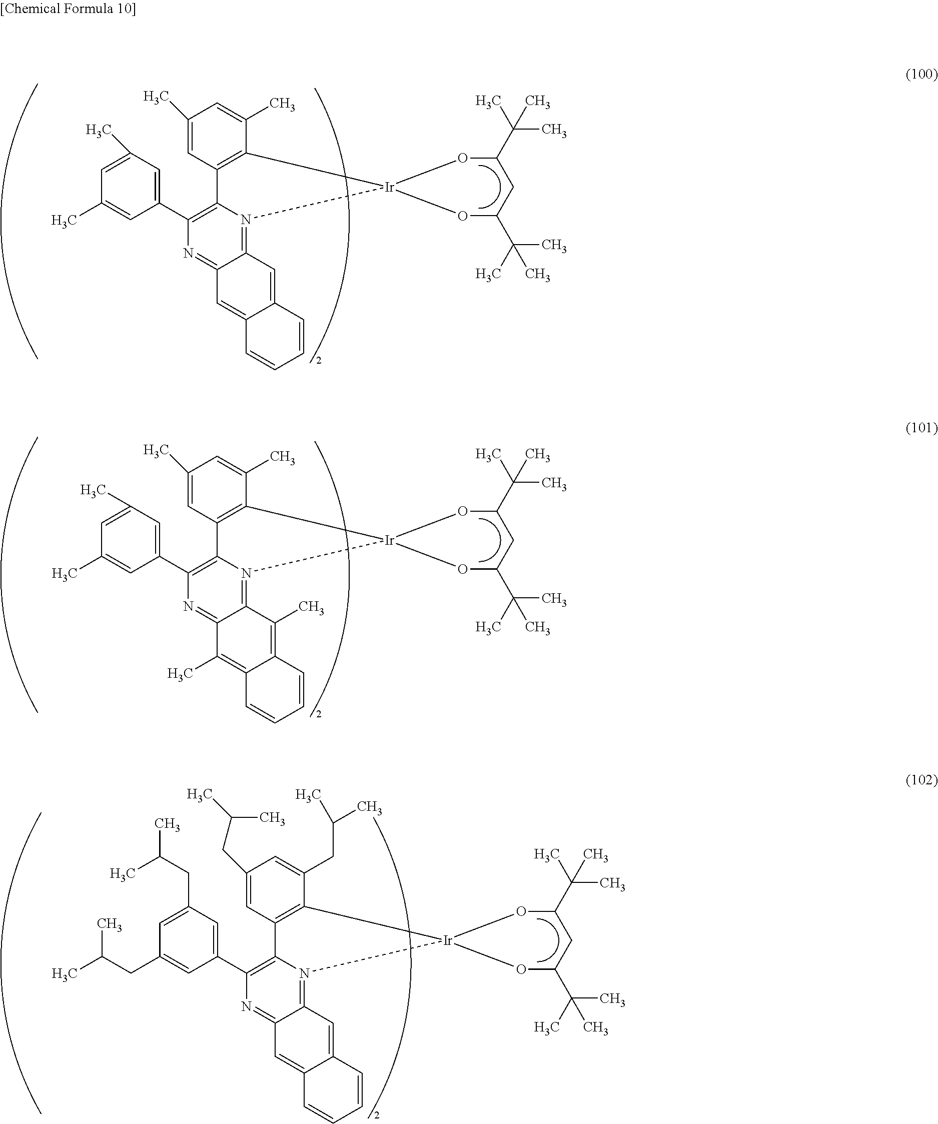

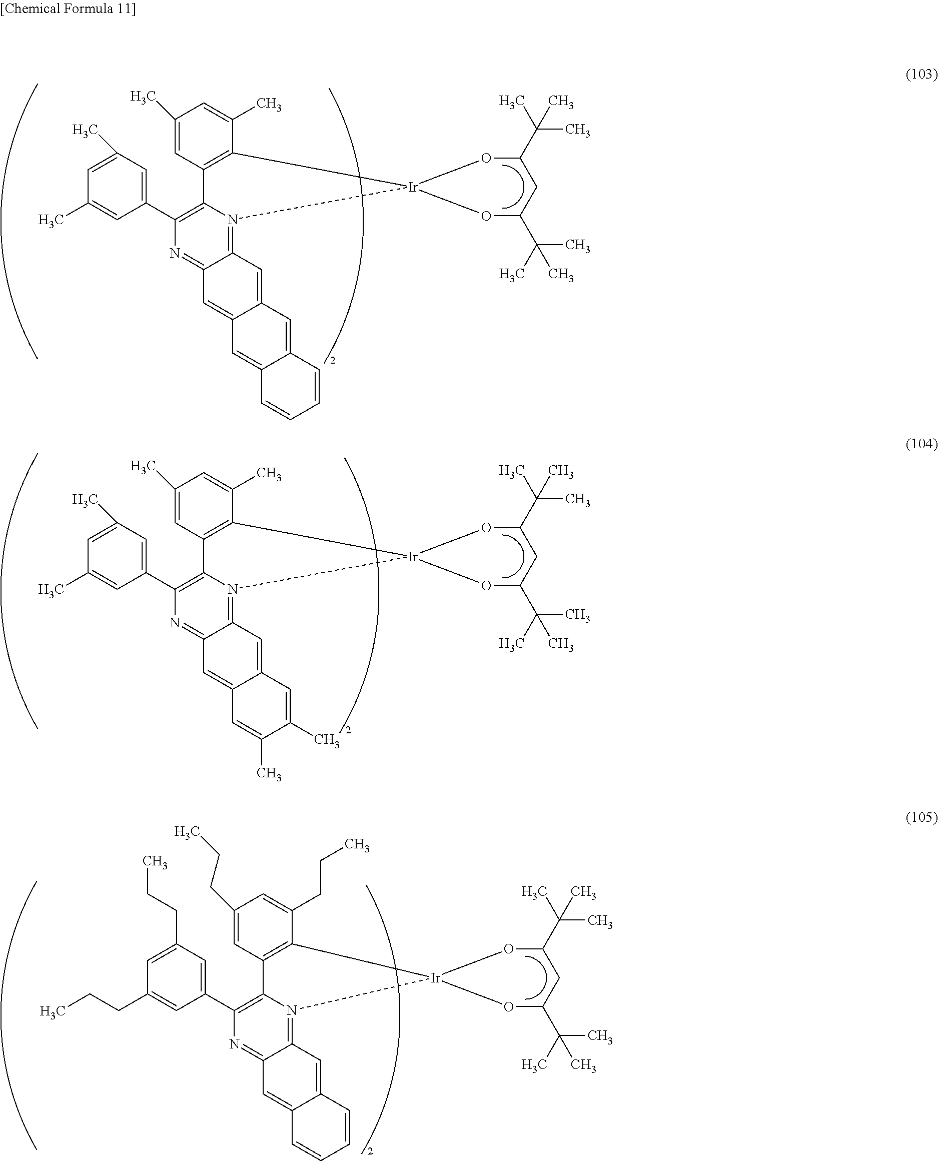

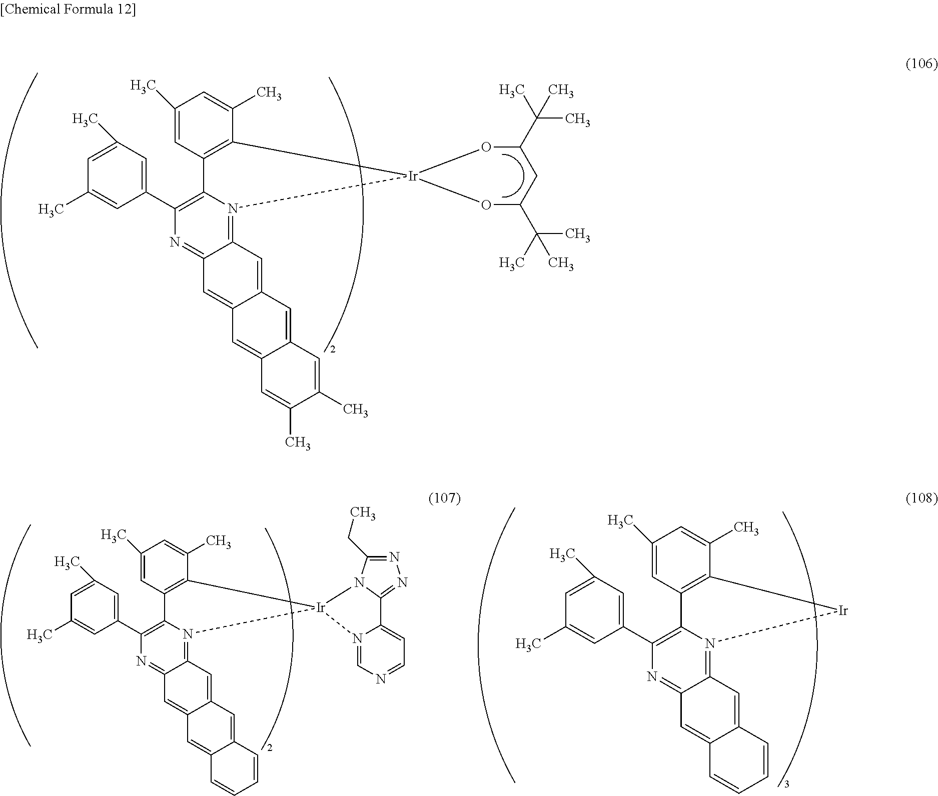

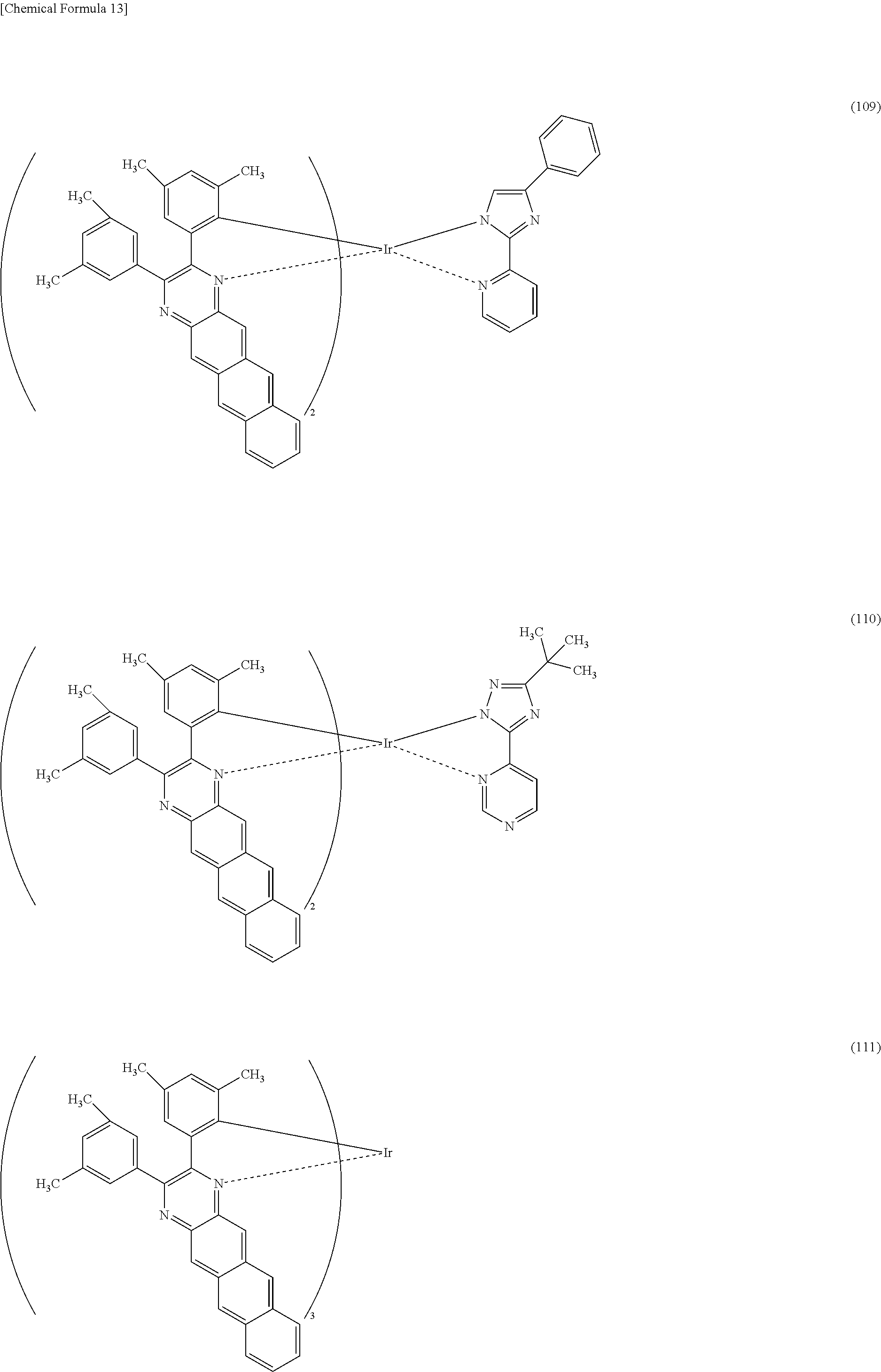

### Method for Synthesizing Organometallic Complex of One Embodiment of the Present Invention

A variety of reactions can be employed as a method for synthesizing the organometallic complex of one embodiment of the present invention. An example of a method for synthesizing the organometallic complex represented by General Formula (G1) is described below.

First, an example of a method for synthesizing the organic compound represented by General Formula (G0) is described, and then, a method for synthesizing the organometallic complex represented by General Formula (G1) with the use of the organic compound represented by General Formula (G0) is described. Note that the case where n in General Formula (G1) is 2 (the organometallic complex represented by General Formula (G1-1)) and the case where n in General Formula (G1) is 3 (the organometallic complex represented by General Formula (G1-2)) are separately described below. Note that the method for synthesizing the organometallic complex of one embodiment of the present invention is not limited to the synthesis methods below.

### Example of Method for Synthesizing Organic Compound Represented by General Formula (G0)

The organic compound represented by General Formula (G0) is a type of quinoxaline derivative and is the organic compound of one embodiment of the present invention. The organic compound represented by General Formula (G0) can be synthesized by any one of three Synthesis Schemes (A-1), (A-1′), and (A-1″) shown below, for example.

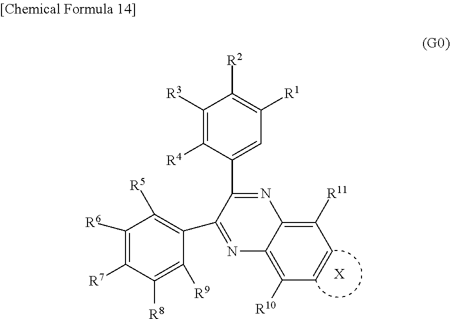

In General Formula (G0) and Synthesis Schemes (A-1), (A-1′), and (A-1″) described below, each of R¹ to R¹¹ independently represents hydrogen or an alkyl group having 1 to 6 carbon atoms; at least two of R¹ to R⁴ represent an alkyl group having 1 to 6 carbon atoms; at least two of R⁵ to R⁹ represent an alkyl group having 1 to 6 carbon atoms; and X represents a substituted or unsubstituted benzene ring or naphthalene ring.

For example, the organic compound represented by General Formula (G0) can be obtained in such a manner that a halogenated benzene derivative (A1) is lithiated with alkyllithium or the like and is reacted with a quinoxaline derivative (A2), as shown in Synthesis Scheme (A-1). In Synthesis Scheme (A-1), Z¹ represents a halogen.

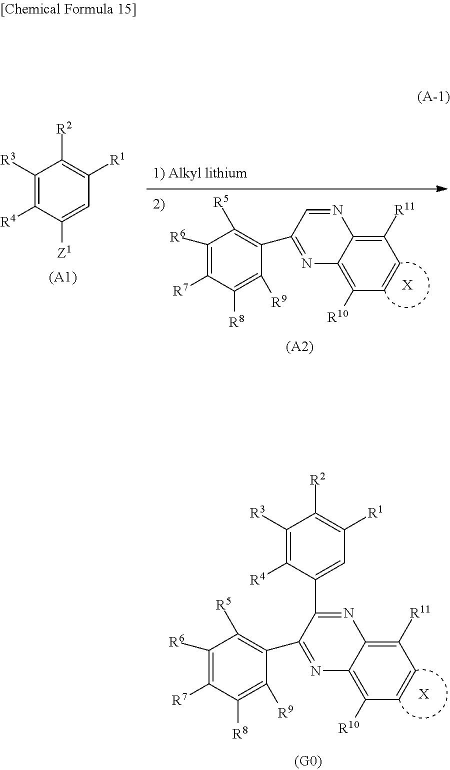

Alternatively, the organic compound represented by General Formula (G0) can be obtained by coupling of a boronic acid (A1′) of a benzene derivative and a halide (A^{2′}) of quinoxaline, as shown in Synthesis Scheme (A-1′). In Synthesis Scheme (A-1′), Z² represents a halogen.

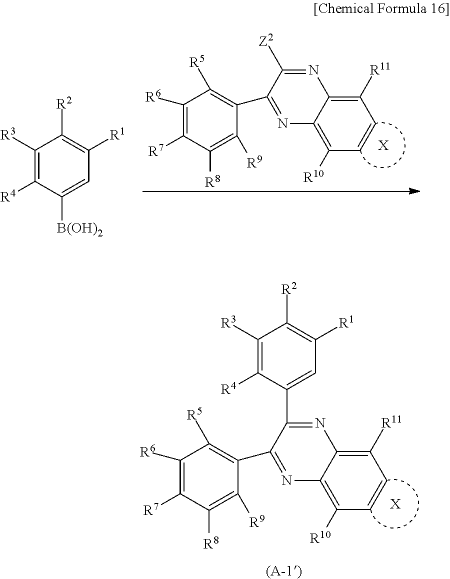

(A¹) (A^{2′}) (G0)

Alternatively, the organic compound represented by General Formula (G0) can be obtained by reacting diketone having benzene derivatives as substituents (A1″) with diamine (A2″), as shown in Synthesis Scheme (A-1″).

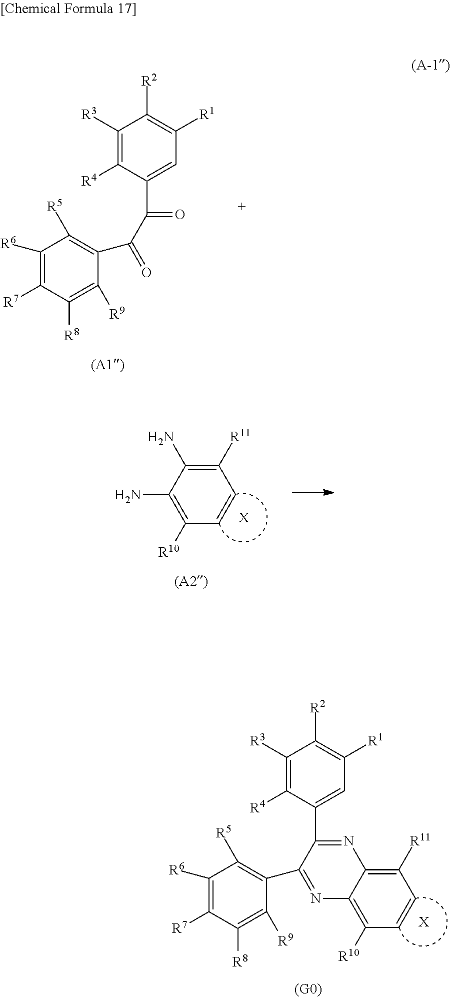

The method for synthesizing the organic compound represented by General Formula (G0) is not limited to the above-described three synthesis methods, and another method may be employed.

Since a variety of the above compounds (A1), (A2), (A1′), (A2′), (A1″), and (A2″) are commercially available or can be obtained by synthesis, various types of the organic compound represented by General Formula (G0) can be synthesized. Thus, the organometallic complex of one embodiment of the present invention is characterized by having numerous variations of ligands.

### Method for Synthesizing Organometallic Complex Represented by General Formula (G1-1)

Next, an example of a method for synthesizing the organometallic complex represented by General Formula (G1-1) is described. The organometallic complex represented by General Formula (G1-1) is the organometallic complex of one embodiment of the present invention and corresponds to the case where n in General Formula (G1) is 2.

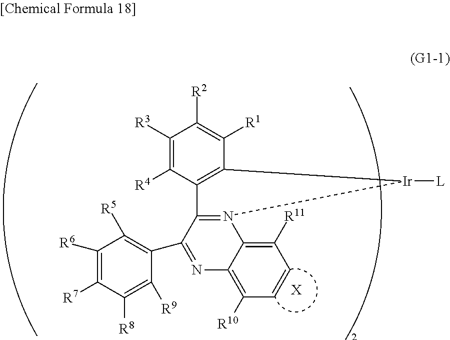

In General Formula (G1-1) and Synthesis Schemes (A-2) and (A-3) described below, each of R¹ to R¹¹ independently represents hydrogen or an alkyl group having 1 to 6 carbon atoms; at least two of R¹ to R⁴ represent an alkyl group having 1 to 6 carbon atoms; at least two of R⁵ to R⁹ represent an alkyl group having 1 to 6 carbon atoms; X represents a substituted or unsubstituted benzene ring or naphthalene ring; and L represents a monoanionic ligand.

First, as shown in Synthesis Scheme (A-2), the organic compound represented by General Formula (G0) and an iridium compound containing a halogen (e.g., iridium chloride, iridium bromide, or iridium iodide) are heated in an inert gas atmosphere using no solvent, an alcohol-based solvent (e.g., glycerol, ethylene glycol, 2-methoxyethanol, or 2-ethoxyethanol) alone, or a mixed solvent of water and one or more of the alcohol-based solvents, whereby the dinuclear complex represented by General Formula (B) can be obtained. There is no particular limitation on a heating means, and an oil bath, a sand bath, or an aluminum block may be used. Alternatively, microwaves can be used as the heating means.

The dinuclear complex represented by General Formula (B) is a type of organometallic complex having a halogen-bridged structure and is a dinuclear complex of one embodiment of the present invention.

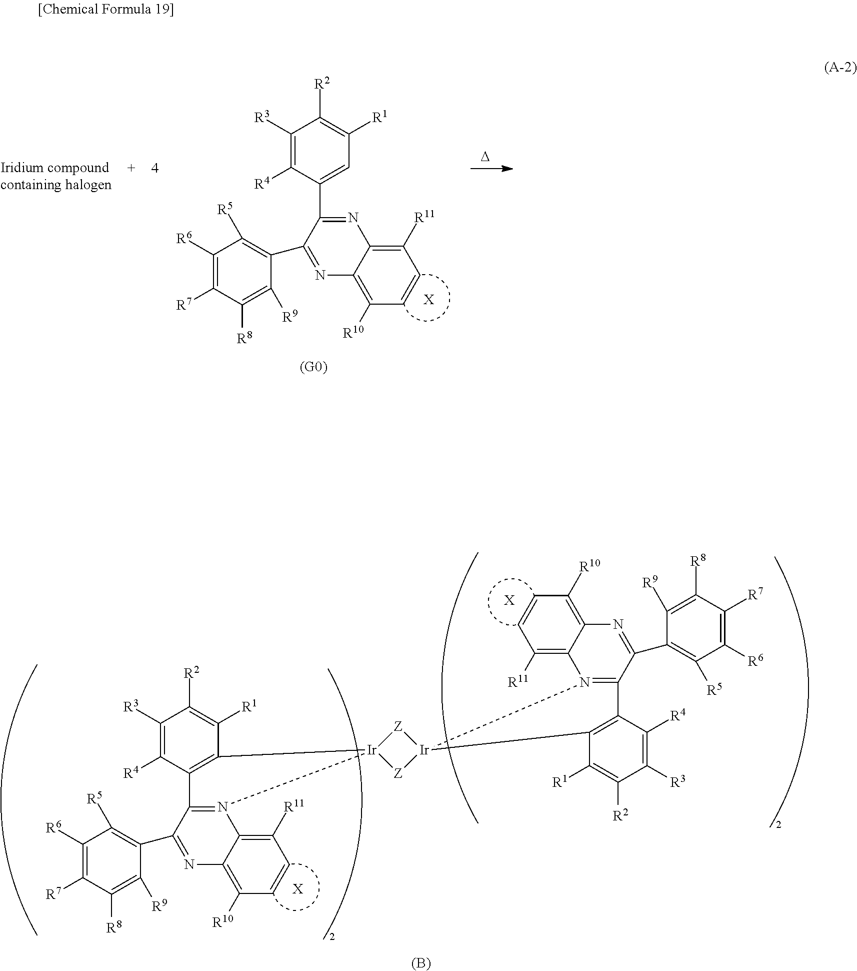

Furthermore, as shown in Synthesis Scheme (A-3), the dinuclear complex represented by General Formula (B) and HL that is a material of the monoanionic ligand are reacted in an inert gas atmosphere, whereby a proton of HL is removed and L coordinates to the central metal (Ir); thus, the organometallic complex represented by General Formula (G1-1) can be obtained. There is no particular limitation on a heating means, and an oil bath, a sand bath, or an aluminum block may be used. Alternatively, microwaves can be used as the heating means.

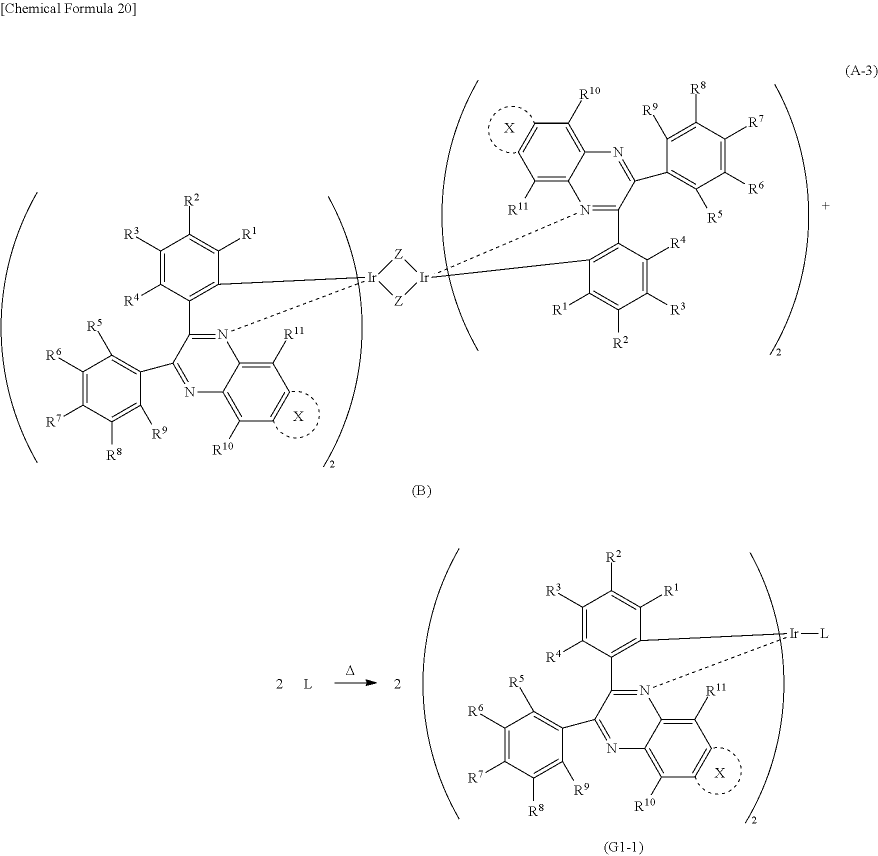

In Synthesis Schemes (A-2) and (A-3), Z represents a halogen.

### Method for Synthesizing Organometallic Complex Represented by General Formula (G1-2)

Next, an example of a method for synthesizing the organometallic complex represented by General Formula (G1-2) is described. The organometallic complex represented by General Formula (G1-2) is the organometallic complex of one embodiment of the present invention and corresponds to the case where n in General Formula (G1) is 3.

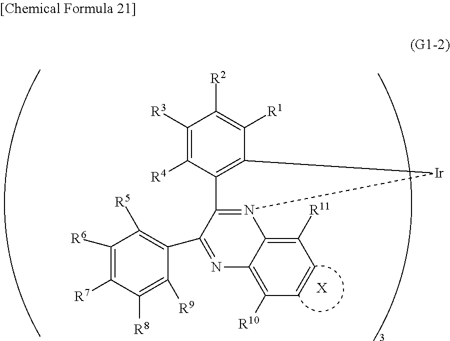

In General Formula (G1-2) and Synthesis Scheme (A-4) described below, each of R¹ to R¹¹ independently represents hydrogen or an alkyl group having 1 to 6 carbon atoms; at least two of R¹ to R⁴ represent an alkyl group having 1 to 6 carbon atoms; at least two of R⁵ to R⁹ represent an alkyl group having 1 to 6 carbon atoms; and X represents a substituted or unsubstituted benzene ring or naphthalene ring.

The organometallic complex represented by General Formula (G1-2) can be obtained in such a manner that, as shown in Synthesis Scheme (A-4), an iridium compound containing a halogen (e.g., iridium chloride hydrate, iridium bromide, iridium iodide, iridium acetate, or ammonium hexachloroiridate) or an organoiridium complex (e.g., an acetylacetonato complex, a diethyl sulfide complex, a di-μ-chloro-bridged dinuclear complex, or a di-μ-hydroxo-bridged dinuclear complex) is mixed with the organic compound represented by General Formula (G0), the mixture is dissolved in an alcohol-based solvent (e.g., glycerol, ethylene glycol, 2-methoxyethanol, or 2-ethoxyethanol) or not dissolved in any solvent, and then heating is performed.

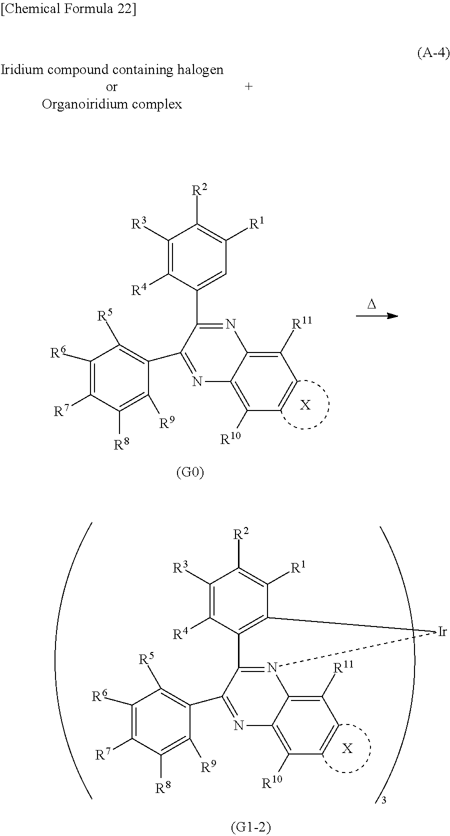

Although the method for synthesizing the organometallic complex of one embodiment of the present invention is described above, the present invention is not limited thereto and synthesis may be performed by any other synthesis method.

As above, the organometallic complex of one embodiment of the present invention emits near-infrared light and has high sublimability, and thus is suitable for a light-emitting material and a light-emitting device material that emit near-infrared light. The use of the organometallic complex of one embodiment of the present invention can increase the emission efficiency of a light-emitting device that emits near-infrared light. Moreover, the use of the organometallic complex of one embodiment of the present invention can increase the reliability of a light-emitting device that emits near-infrared light.

This embodiment can be combined with the other embodiments as appropriate. In this specification, in the case where a plurality of structure examples are shown in one embodiment, the structure examples can be combined as appropriate.

### Embodiment 2

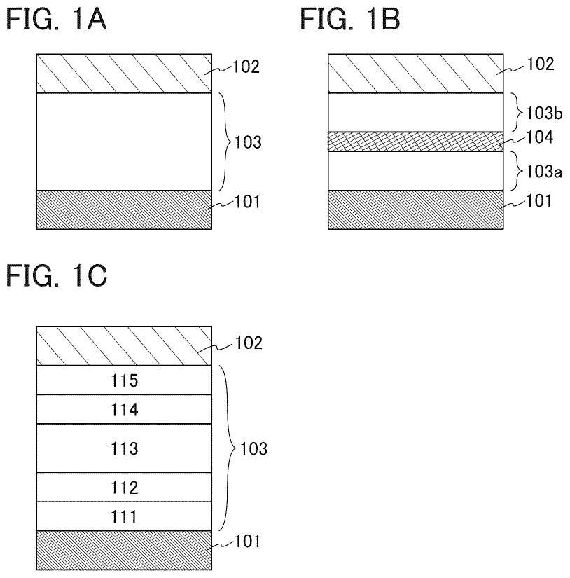

In this embodiment, a light-emitting device of one embodiment of the present invention and a light-emitting apparatus of one embodiment of the present invention will be described with reference to FIG. 1 to FIG. 3.

The light-emitting device of one embodiment of the present invention contains a light-emitting organic compound in a light-emitting layer. The maximum peak wavelength (also referred to as wavelength at which the peak intensity is the highest) of light emitted from the light-emitting organic compound is greater than or equal to 760 nm and less than or equal to 900 nm, preferably greater than or equal to 780 nm and preferably less than or equal to 880 nm.

The light-emitting device of one embodiment of the present invention can be formed into a film shape and is easily increased in area, and thus can be used as a planar light source that emits near-infrared light.

The light-emitting organic compound preferably exhibits phosphorescence, in which case the emission efficiency of the light-emitting device can be increased. The light-emitting organic compound is particularly preferably an organometallic complex having a metal-carbon bond. In particular, the light-emitting organic compound is further preferably a cyclometalated complex. Furthermore, the light-emitting organic compound is preferably an orthometalated complex. These organic compounds are likely to exhibit phosphorescence, and thus can increase the emission efficiency of the light-emitting device. Consequently, the light-emitting device of one embodiment of the present invention is preferably a phosphorescent device that exhibits phosphorescence.

The organometallic complex having a metal-carbon bond is suitable for the light-emitting organic compound because of its higher emission efficiency and higher chemical stability than a porphyrin-based compound and the like.

In the case where the light-emitting organic compound is used as a guest material and another organic compound is used as a host material in a light-emitting layer, when a deep trough appears (a portion with a low intensity appears) in the absorption spectrum of the light-emitting organic compound, energy is not transferred smoothly from the host material to the guest material and the energy transfer efficiency is lowered in some cases that depend on the value of excitation energy of the host material. Here, in the absorption spectrum of the organometallic complex having a metal-carbon bond, many absorption bands, such as an absorption band derived from triplet MLCT (Metal to Ligand Charge Transfer) transition, an absorption band derived from singlet MLCT transition, and an absorption band derived from triplet π-π* transition, overlap each other; hence, a deep trough is less likely to appear in the absorption spectrum. Thus, the range of the value of excitation energy of the material that can be used as the host material can be widened, and the range of choices for the host material can be widened.

In addition, the light-emitting organic compound is preferably an iridium complex. For example, the light-emitting organic compound is preferably a cyclometalated complex using iridium as the central metal. Since the iridium complex has higher chemical stability than a platinum complex and the like, the use of the iridium complex as the light-emitting organic compound can increase the reliability of the light-emitting device. In terms of such stability, a cyclometalated complex of iridium is preferable, and an orthometalated complex of iridium is further preferable.

From the viewpoint of obtaining near-infrared light emission, the ligand of the above organometallic complex preferably has a structure in which a condensed heteroaromatic ring including 2 to 5 rings is coordinated to a metal. The condensed heteroaromatic ring preferably includes 3 or more rings. Moreover, the condensed heteroaromatic ring preferably includes 4 or less rings. As the number of rings included in the condensed heteroaromatic ring increases, the LUMO level can be lower and the wavelength of light emitted from the organometallic complex can be longer. Meanwhile, as the number of condensed heteroaromatic rings decreases, the sublimability can be increased. Consequently, by employing a condensed heteroaromatic ring including 2 to 5 rings, the LUMO level of the ligand is adequately lowered, and the wavelength of light that is emitted from the organometallic complex and derived from the (triplet) MLCT transition can be increased to the near-infrared wavelength while high sublimability is maintained. In addition, as the number of nitrogen atoms (N) included in the condensed heteroaromatic ring increases, the LUMO level can be lower. Therefore, the number of nitrogen atoms (N) included in the condensed heteroaromatic ring is preferably two or more, particularly preferably two.

### Structure Example of Light-Emitting Device

### Basic Structure of Light-Emitting Device

FIG. 1A to FIG. 1C illustrate examples of light-emitting devices including an EL layer between a pair of electrodes.

The light-emitting device illustrated in FIG. 1A has a structure in which an EL layer 103 is provided between a first electrode 101 and a second electrode 102 (a single structure). The EL layer 103 includes at least a light-emitting layer.

A light-emitting device may include a plurality of EL layers between a pair of electrodes. FIG. 1B illustrates a light-emitting device having a tandem structure in which two EL layers (an EL layer 103a and an EL layer 103b) are provided between a pair of electrodes and a charge-generation layer 104 is provided between the two EL layers. The light-emitting device having a tandem structure can be driven at low voltage and have low power consumption.

The charge-generation layer 104 has a function of injecting electrons into one of the EL layer 103a and the EL layer 103b and injecting holes into the other of the EL layers when voltage is applied to the first electrode 101 and the second electrode 102. Thus, when voltage is applied in FIG. 1B such that the potential of the first electrode 101 is higher than that of the second electrode 102, the charge-generation layer 104 injects electrons into the EL layer 103a and injects holes into the EL layer 103b.

Note that in terms of light extraction efficiency, the charge-generation layer 104 preferably transmits near-infrared light (specifically, the near-infrared light transmittance of the charge-generation layer 104 is preferably 40% or higher). Furthermore, the charge-generation layer 104 functions even when having lower conductivity than the first electrode 101 and the second electrode 102.

FIG. 1C illustrates an example of a stacked-layer structure of the EL layer 103. In this embodiment, the case where the first electrode 101 functions as an anode and the second electrode 102 functions as a cathode is described as an example. The EL layer 103 has a structure in which a hole-injection layer 111, a hole-transport layer 112, a light-emitting layer 113, an electron-transport layer 114, and an electron-injection layer 115 are stacked in this order over the first electrode 101. Each of the hole-injection layer 111, the hole-transport layer 112, the light-emitting layer 113, the electron-transport layer 114, and the electron-injection layer 115 may have a single-layer structure or a stacked-layer structure. Note that in the case where a plurality of EL layers are provided as in the tandem structure illustrated in FIG. 1B, each of the EL layers can have a stacked-layer structure similar to that of the EL layer 103 illustrated in FIG. 1C. When the first electrode 101 serves as a cathode and the second electrode 102 serves as an anode, the stacking order is reversed.

The light-emitting layer 113 contains a light-emitting substance and a plurality of substances in appropriate combination, whereby fluorescence or phosphorescence with a desired wavelength can be obtained. Furthermore, the light-emitting layer 113 may be a stack of layers having different emission wavelengths. In that case, the light-emitting substance and other substances are different between the stacked light-emitting layers. The EL layer 103a and the EL layer 103b illustrated in FIG. 1B may be configured to exhibit different wavelengths. Also in that case, the light-emitting substance and other substances are different between the light-emitting layers.

The light-emitting device of one embodiment of the present invention may be configured such that light obtained from the EL layer is resonated between the pair of electrodes in order to intensify the light. For example, when the first electrode 101 is formed as a reflective electrode and the second electrode 102 is formed as a transflective electrode (an electrode having properties of transmitting and reflecting near-infrared light) in FIG. 1C to form a micro optical resonator (microcavity) structure, light obtained from the EL layer 103 can be intensified.

Note that in the case where the first electrode 101 of the light-emitting device is a reflective electrode having a stacked-layer structure of a conductive film having a property of reflecting near-infrared light and a conductive film having a property of transmitting near-infrared light, optical adjustment can be performed by controlling the thicknesses of the conductive film having the transmitting property. Specifically, when the wavelength of light obtained from the light-emitting layer 113 is λ, the distance between the first electrode 101 and the second electrode 102 is preferably adjusted to around mλ/2 (m is a natural number).

To amplify desired light (wavelength: λ) obtained from the light-emitting layer 113, the optical distance from the first electrode 101 to a region where the desired light is obtained in the light-emitting layer 113 (a light-emitting region) and the optical distance from the second electrode 102 to the region where the desired light is obtained in the light-emitting layer 113 (the light-emitting region) are preferably adjusted to around (2m′+1)λ/4 (m′ is a natural number). Here, the light-emitting region refers to a region where holes and electrons are recombined in the light-emitting layer 113.

By performing such optical adjustment, the spectrum of light obtained from the light-emitting layer 113 can be narrowed, and light with a desired wavelength can be obtained.

Note that in the above case, the optical distance between the first electrode 101 and the second electrode 102 is, to be exact, the total thickness from a reflective region in the first electrode 101 to a reflective region in the second electrode 102. However, it is difficult to precisely determine the reflective regions in the first electrode 101 and the second electrode 102; thus, it is assumed that the above effect can be sufficiently obtained with given positions in the first electrode 101 and the second electrode 102 being supposed to be reflective regions. Furthermore, the optical distance between the first electrode 101 and the light-emitting layer from which the desired light is obtained is, to be exact, the optical distance between the reflective region in the first electrode 101 and the light-emitting region in the light-emitting layer from which the desired light is obtained. However, it is difficult to precisely determine the reflective region in the first electrode 101 and the light-emitting region in the light-emitting layer from which the desired light is obtained; thus, it is assumed that the above effect can be sufficiently obtained with a given position in the first electrode 101 being supposed to be the reflective region and a given position in the light-emitting layer from which the desired light is obtained being supposed to be the light-emitting region.

At least one of the first electrode 101 and the second electrode 102 has a property of transmitting near-infrared light. The transmissivity of near-infrared light of the electrode having a property of transmitting near-infrared light is higher than or equal to 40%. In the case where the electrode having a property of transmitting near-infrared light is the above-described transflective electrode, the near-infrared light reflectance of the electrode is higher than or equal to 20%, preferably higher than or equal to 40% and lower than 100%, preferably lower than or equal to 95%, and may be lower than or equal to 80% or lower than or equal to 70%. For example, the near-infrared light reflectance of the electrode is higher than or equal to 20% and lower than or equal to 80%, preferably higher than or equal to 40% and lower than or equal to 70%. The electrode preferably has a resistivity of 1×10⁻² Ωcm or less.

When the first electrode 101 or the second electrode 102 is an electrode having a property of reflecting near-infrared light (a reflective electrode), the near-infrared light reflectance of the reflective electrode is higher than or equal to 40% and lower than or equal to 100%, preferably higher than or equal to 70% and lower than or equal to 100%. This electrode preferably has a resistivity of 1×10⁻² Ωcm or less.

### Specific Structure and Fabrication Method of Light-Emitting Device

Next, a specific structure and a fabrication method of the light-emitting device will be described. Here, the light-emitting device having the single structure illustrated in FIG. 1C is used for the description.

### First Electrode and Second Electrode

As materials for forming the first electrode 101 and the second electrode 102, any of the following materials can be used in an appropriate combination as long as the functions of the electrodes described above can be fulfilled. For example, a metal, an alloy, an electrically conductive compound, a mixture of these, and the like can be used as appropriate. Specific examples include In-Sn oxide (also referred to as ITO), In—Si—Sn oxide (also referred to as ITSO), In—Zn oxide, and In—W—Zn oxide. In addition, it is possible to use a metal such as aluminum (Al), titanium (Ti), chromium (Cr), manganese (Mn), iron (Fe), cobalt (Co), nickel (Ni), copper (Cu), gallium (Ga), zinc (Zn), indium (In), tin (Sn), molybdenum (Mo), tantalum (Ta), tungsten (W), palladium (Pd), gold (Au), platinum (Pt), silver (Ag), yttrium (Y), or neodymium (Nd) or an alloy containing an appropriate combination of any of these metals. It is also possible to use a Group 1 element or a Group 2 element in the periodic table, which is not described above (e.g., lithium (Li), cesium (Cs), calcium (Ca), or strontium (Sr)), a rare earth metal such as europium (Eu) or ytterbium (Yb), an alloy containing an appropriate combination of any of these, graphene, or the like.

Note that when a light-emitting device having a microcavity structure is formed, the first electrode 101 is formed as a reflective electrode and the second electrode 102 is formed as a transflective electrode. Thus, a single layer or stacked layers can be formed using one or more desired conductive materials. Note that the second electrode 102 is formed after formation of the EL layer 103, with the use of a material selected as described above. For fabrication of these electrodes, a sputtering method or a vacuum evaporation method can be used.

When the first electrode 101 is an anode in the light-emitting device illustrated in FIG. 1C, the hole-injection layer 111 and the hole-transport layer 112 are sequentially stacked over the first electrode 101 by a vacuum evaporation method.

### Hole-Injection Layer and Hole-Transport Layer

The hole-injection layer 111 is a layer injecting holes from the first electrode 101 serving as the anode to the EL layer 103, and is a layer containing a material with a high hole-injection property.

As the material with a high hole-injection property, a transition metal oxide such as molybdenum oxide, vanadium oxide, ruthenium oxide, tungsten oxide, or manganese oxide or a phthalocyanine-based compound such as phthalocyanine (abbreviation: H₂Pc) or copper(II) phthalocyanine (abbreviation: CuPc) can be used, for example.

As the material with a high hole-injection property, it is possible to use, for example, an aromatic amine compound such as 4,4′,4″-tris(N,N-diphenylamino)triphenylamine (abbreviation: TDATA), 4,4′,4″-tris[N-(3-methylphenyl)-N-phenylamino]triphenylamine (abbreviation: MTDATA), 4,4′-bis[N-(4-diphenylaminophenyl)-N-phenylamino]biphenyl (abbreviation: DPAB), 4,4′-bis(N-{4-[N-(3-methylphenyl)-N′-phenylamino]phenyl}-N-phenylamino) biphenyl (abbreviation: DNTPD), 1,3,5-tris[N-(4-diphenylaminophenyl)-N-phenylamino]benzene (abbreviation: DPA3B), 3-[N-(9-phenylcarbazol-3-yl)-N-phenylamino]-9-phenylcarbazole (abbreviation: PCzPCA1), 3,6-bis[N-(9-phenylcarbazol-3-yl)-N-phenylamino]-9-phenylcarbazole (abbreviation: PCzPCA2), or 3-[N-(1-naphthyl)-N-(9-phenylcarbazol-3-yl)amino]-9-phenylcarbazole (abbreviation: PCzPCN1).

As the material with a high hole-injection property, it is possible to use, for example, poly(N-vinylcarbazole) (abbreviation: PVK), poly(4-vinyltriphenylamine) (abbreviation: PVTPA), poly[N-(4-{N′-[4-(4-diphenylamino)phenyl]phenyl-N′-phenylamino}phenyl)methacrylamide] (abbreviation: PTPDMA), or poly[N,N′-bis(4-butylphenyl)-N,′N-bis(phenyl)benzidine] (abbreviation: Poly-TPD). Alternatively, it is also possible to use, for example, a high molecular compound to which acid is added, such as poly (3,4-ethylenedioxythiophene)/poly(styrenesulfonic acid) (abbreviation: PEDOT/PSS) or polyaniline/poly(styrenesulfonic acid) (PAni/PSS).

As the material with a high hole-injection property, a composite material containing a hole-transport material and an acceptor material (an electron-accepting material) can also be used. In this case, the acceptor material extracts electrons from the hole-transport material, so that holes are generated in the hole-injection layer 111 and the holes are injected into the light-emitting layer 113 through the hole-transport layer 112. Note that the hole-injection layer 111 may be formed using a single layer of a composite material containing a hole-transport material and an acceptor material, or may be formed using a stack including a layer of a hole-transport material and a layer of an acceptor material.

The hole-transport layer 112 is a layer transporting holes, which are injected from the first electrode 101 by the hole-injection layer 111, to the light-emitting layer 113. The hole-transport layer 112 is a layer containing a hole-transport material. It is particularly preferable that the highest occupied molecular orbital level (HOMO level) of the hole-transport material used in the hole-transport layer 112 be the same as or close to the HOMO level of the hole-injection layer 111.

As the acceptor material used for the hole-injection layer 111, an oxide of a metal belonging to any of Group 4 to Group 8 of the periodic table can be used. Specific examples include molybdenum oxide, vanadium oxide, niobium oxide, tantalum oxide, chromium oxide, tungsten oxide, manganese oxide, and rhenium oxide. Among these, molybdenum oxide is particularly preferable since it is stable in the air, has a low hygroscopic property, and is easy to handle. Alternatively, organic acceptors such as a quinodimethane derivative, a chloranil derivative, and a hexaazatriphenylene derivative can be used. Examples of materials having an electron-withdrawing group (halogen group or cyano group) include 7,7,8,8-tetracyano-2,3,5,6-tetrafluoroquinodimethane (abbreviation: F₄-TCNQ), chloranil, 2,3,6,7,10,11-hexacyano-1,4,5,8,9,12-hexaazatriphenylene (abbreviation: HAT-CN), and 1,3,4,5,7,8-hexafluorotetracyano-naphthoquinodimethane (abbreviation: F6-TCNNQ). A compound in which electron-withdrawing groups are bonded to a condensed aromatic ring having a plurality of hetero atoms, such as HAT-CN, is particularly preferable because it is thermally stable. A [3]radialene derivative including an electron-withdrawing group (in particular, a cyano group or a halogen group such as a fluoro group) has a very high electron-accepting property and thus is preferred; specific examples include α,α′,α″-1,2,3-cyclopropanetriylidenetris[4-cyano-2,3,5,6-tetrafluorobenzeneacetonitrile], α,α′,α″-1,2,3-cyclopropanetriylidenetris[2,6-dichloro-3,5-difluoro-4-(trifluoromethyl)benzeneacetonitrile], and α,α′,α″-1,2,3-cyclopropanetriylidenetris[2,3,4,5,6-pentafluorobenzeneacetonitrile].

The hole-transport materials used for the hole-injection layer 111 and the hole-transport layer 112 are preferably substances with a hole mobility of greater than or equal to 10⁻⁶ cm²/Vs. Note that other substances can also be used as long as they have a property of transporting more holes than electrons.

As the hole-transport material, materials having a high hole-transport property, such as a π-electron rich heteroaromatic compound (e.g., a carbazole derivative, a thiophene derivative, and a furan derivative) and an aromatic amine (a compound having an aromatic amine skeleton), are preferable.

Examples of the carbazole derivative (a compound having a carbazole skeleton) include a bicarbazole derivative (e.g., a 3,3′-bicarbazole derivative) and an aromatic amine having a carbazolyl group.

Specific examples of the bicarbazole derivative (e.g., a 3,3′-bicarbazole derivative) include 3,3′-bis(9-phenyl-9H-carbazole) (abbreviation: PCCP), 9,9′-bis(1,1′-biphenyl-4-yl)-3,3′-bi-9H-carbazole, 9,9′-bis(1,1′-biphenyl-3-yl)-3,3′-bi-9H-carbazole, 9-(1,1′-biphenyl-3-yl)-9′-(1,1′-biphenyl-4-yl)-9H,9′H-3,3′-bicarbazole (abbreviation: mBPCCBP), and 9-(2-naphthyl)-9′-phenyl-9H,9′H-3,3′-bicarbazole (abbreviation: βNCCP).

Specific examples of the aromatic amine having a carbazolyl group include 4-phenyl-4′-(9-phenyl-9H-carbazol-3-yl)triphenylamine (abbreviation: PCBA1BP), N-(4-biphenyl)-N-(9,9-dimethyl-9H-fluoren-2-yl)-9-phenyl-9H-carbazol-3-amine (abbreviation: PCBiF), N-(1,1′-biphenyl-4-yl)-N-[4-(9-phenyl-9H-carbazol-3-yl)phenyl]-9,9-dimethyl-9H-fluoren-2-amine (abbreviation: PCBBiF), 4,4′-diphenyl-4″-(9-phenyl-9H-carbazol-3-yl)triphenylamine 4-(1-naphthyl)-4′-(9-phenyl-9H-carbazol-3-yl)triphenylamine (abbreviation: PCBBi1BP), (abbreviation: PCBANB), 4,4′-di(1-naphthyl)-4″-(9-phenyl-9H-carbazol-3-yl)triphenylamine (abbreviation: PCBNBB), 4-phenyldiphenyl-(9-phenyl-9H-carbazol-3-yl)amine (abbreviation: PCA1BP), N,N′-bis(9-phenylcarbazol-3-yl)-N,N′-diphenylbenzene-1,3-diamine (abbreviation: PCA2B), N,N′, N″-triphenyl-N,N′,N″-tris(9-phenylcarbazol-3-yl)benzene-1,3,5-triamine (abbreviation: PCA3B), 9,9-dimethyl-N-phenyl-N-[4-(9-phenyl-9H-carbazol-3-yl)phenyl]fluoren-2-amine (abbreviation: PCBAF), N-phenyl-N-[4-(9-phenyl-9H-carbazol-3-yl)phenyl]spiro-9,9′-bifluoren-2-amine (abbreviation: PCBASF), PCzPCA1, PCzPCA2,PCzPCN1, 3-[N-(4-diphenylaminophenyl)-N-phenylamino]-9-phenylcarbazole (abbreviation: PCzDPA1), 3,6-bis[N-(4-diphenylaminophenyl)-N-phenylamino]-9-phenylcarbazole (abbreviation: PCzDPA2), 3,6-bis[N-(4-diphenylaminophenyl)-N-(1-naphthyl)amino]-9-phenylcarbazole (abbreviation: PCzTPN2), 2-[N-(9-phenylcarbazol-3-yl)-N-phenylamino]spiro-9,9′-bifluorene (abbreviation: PCASF), N-[4-(9H-carbazol-9-yl)phenyl]-N-(4-phenyl)phenylaniline (abbreviation: YGA1BP), N,N′-bis[4-(carbazol-9-yl)phenyl]-N,N′-diphenyl-9,9-dimethylfluorene-2,7-diamine (abbreviation: YGA2F), and 4,4′,4″-tris(carbazol-9-yl)triphenylamine (abbreviation: TCTA).

In addition to the above, other examples of the carbazole derivative include 3-[4-(9-phenanthryl)-phenyl]-9-phenyl-9H-carbazole (abbreviation: PCPPn), 3-[4-(1-naphthyl)-phenyl]-9-phenyl-9H-carbazole (abbreviation: PCPN), 1,3-bis(N-carbazolyl)benzene (abbreviation: mCP), 4,4′-di(N-carbazolyl)biphenyl (abbreviation: CBP), 3,6-bis(3,5-diphenylphenyl)-9-phenylcarbazole (abbreviation: CzTP), 1,3,5-tris[4-(N-carbazolyl)phenyl]benzene (abbreviation: TCPB), and 9-[4-(10-phenyl-9-anthracenyl)phenyl]-9H-carbazole (abbreviation: CzPA).

Specific examples of the thiophene derivative (a compound having a thiophene skeleton) and the furan derivative (a compound having a furan skeleton) include compounds having a thiophene skeleton, such as 4,4′,4″-(benzene-1,3,5-triyl)tri(dibenzothiophene) (abbreviation: DBT3P-II), 2,8-diphenyl-4-[4-(9-phenyl-9H-fluoren-9-yl)phenyl]dibenzothiophene (abbreviation: DBTFLP-III), and 4-[4-(9-phenyl-9H-fluoren-9-yl)phenyl]-6-phenyldibenzothiophene (abbreviation: DBTFLP-IV), 4,4′,4″-(benzene-1,3,5-triyl)tri(dibenzofuran) (abbreviation: DBF3P-II), and 4-{3-[3-(9-phenyl-9H-fluoren-9-yl)phenyl]phenyl}dibenzofuran (abbreviation: mmDBFFLBi-II).

Specific examples of the aromatic amine include 4,4′-bis[N-(1-naphthyl)-N-phenylamino]biphenyl (abbreviation: NPB or α-NPD), N,N′-bis(3-methylphenyl)-N,N′-diphenyl-[1,1′-biphenyl]-4,4′-diamine (abbreviation: TPD), 4,4′-bis[N-(spiro-9,9′-bifluoren-2-yl)-N-phenylamino]biphenyl (abbreviation: BSPB), 4-phenyl-4′-(9-phenylfluoren-9-yl)triphenylamine (abbreviation: BPAFLP), 4-phenyl-3′-(9-phenylfluoren-9-yl)triphenylamine (abbreviation: mBPAFLP), N-(9,9-dimethyl-9H-fluoren-2-yl)-N-{9,9-dimethyl-2-[N′-phenyl-N′-(9,9-dimethyl-9H-fluoren-2-yl)amino]-9H-fluoren-7-yl}phenylamine (abbreviation: DFLADFL), N-(9,9-dimethyl-2-diphenylamino-9H-fluoren-7-yl)diphenylamine (abbreviation: DPNF), 2-[N-(4-diphenylaminophenyl)-N-phenylamino]spiro-9,9′-bifluorene (abbreviation: DPASF), 2,7-bis[N-(4-diphenylaminophenyl)-N-phenylamino]spiro-9,9′-bifluorene (abbreviation: DPA2SF), 4,4′,4″-tris[N-(1-naphthyl)-N-phenylamino]triphenylamine (abbreviation: 1′-TNATA), TDATA, m-MTDATA, N,N′-di(p-tolyl)-N,N′-diphenyl-p-phenylenediamine (abbreviation: DTDPPA), DPAB, DNTPD, and DPA3B.

As the hole-transport material, a high molecular compound such as PVK, PVTPA, PTPDMA, or Poly-TPD can also be used.

The hole-transport material is not limited to the above examples, and one of or a combination of various known materials can be used as the hole-transport material in the hole-injection layer 111 and the hole-transport layer 112.

In the light-emitting device illustrated in FIG. 1C, the light-emitting layer 113 is formed over the hole-transport layer 112 by a vacuum evaporation method.

### Light-Emitting Layer

The light-emitting layer 113 is a layer containing a light-emitting substance.

The light-emitting device of one embodiment of the present invention contains a light-emitting organic compound as the light-emitting substance. The light-emitting organic compound emits near-infrared light. Specifically, the maximum peak wavelength of light emitted from the light-emitting organic compound is greater than 780 nm and less than or equal to 900 nm.

As the light-emitting organic compound, any of the organometallic complexes described in Embodiment 1 can be used, for example. Alternatively, as the light-emitting organic compound, any of organometallic complexes that will be described later in Examples can be used.

The light-emitting layer 113 can contain one or more kinds of light-emitting substances.

The light-emitting layer 113 may contain one or more kinds of organic compounds (e.g., a host material and an assist material) in addition to the light-emitting substance (guest material). As the one or more kinds of organic compounds, one or both of the hole-transport material and the electron-transport material described in this embodiment can be used. Alternatively, as the one or more kinds of organic compounds, a bipolar material may be used.

There is no particular limitation on the light-emitting substance that can be used for the light-emitting layer 113, and it is possible to use a light-emitting substance that converts singlet excitation energy into light in the near-infrared light range or a light-emitting substance that converts triplet excitation energy into light in the near-infrared light range.

As an example of the light-emitting substance that converts singlet excitation energy into light, a substance that exhibits fluorescence (a fluorescent material) can be given; examples include a pyrene derivative, an anthracene derivative, a triphenylene derivative, a fluorene derivative, a carbazole derivative, a dibenzothiophene derivative, a dibenzofuran derivative, a dibenzoquinoxaline derivative, a quinoxaline derivative, a pyridine derivative, a pyrimidine derivative, a phenanthrene derivative, and a naphthalene derivative.

Examples of the light-emitting substance that converts triplet excitation energy into light include a substance that exhibits phosphorescence (a phosphorescent material) and a thermally activated delayed fluorescence (TADF) material that exhibits thermally activated delayed fluorescence.

Examples of the phosphorescent material include an organometallic complex (particularly an iridium complex) having a 4H-triazole skeleton, a 1H-triazole skeleton, an imidazole skeleton, a pyrimidine skeleton, a pyrazine skeleton, or a pyridine skeleton; an organometallic complex (particularly an iridium complex) having a phenylpyridine skeleton including an electron-withdrawing group as a ligand; a platinum complex; and a rare earth metal complex.

The light-emitting device of one embodiment of the present invention may contain a light-emitting substance other than the light-emitting substance that emits near-infrared light. For example, the light-emitting device of one embodiment of the present invention may contain a light-emitting substance that emits visible light (of red, blue, green, or the like) in addition to the light-emitting substance that emits near-infrared light.

As the organic compounds (e.g., the host material and the assist material) used in the light-emitting layer 113, one or more kinds of substances having a larger energy gap than the light-emitting substance can be used.

In the case where the light-emitting substance used in the light-emitting layer 113 is a fluorescent material, an organic compound used in combination with the light-emitting substance is preferably an organic compound that has a high energy level in a singlet excited state and has a low energy level in a triplet excited state.

In terms of a preferable combination with the light-emitting substance (the fluorescent material or the phosphorescent material), specific examples of the organic compounds are shown below though some of them overlap the specific examples shown above.

In the case where the light-emitting substance is a fluorescent material, examples of the organic compound that can be used in combination with the light-emitting substance include condensed polycyclic aromatic compounds, such as an anthracene derivative, a tetracene derivative, a phenanthrene derivative, a pyrene derivative, a chrysene derivative, and a dibenzo[g,p]chrysene derivative.

Specific examples of the organic compound (the host material) used in combination with the fluorescent material include 9-phenyl-3-[4-(10-phenyl-9-anthryl)phenyl]-9H-carbazole (abbreviation: PCzPA), 3,6-diphenyl-9-[4-(10-phenyl-9-anthryl)phenyl]-9H-carbazole (abbreviation: DPCzPA), PCPN, 9,10-diphenylanthracene (abbreviation: DPAnth), N,N-diphenyl-9-[4-(10-phenyl-9-anthryl)phenyl]-9H-carbazol-3-amine (abbreviation: CzA1PA), 4-(10-phenyl-9-anthryl)triphenylamine (abbreviation: DPhPA), 4-(9H-carbazol-9-yl)-4′-(10-phenyl-9-anthryl)triphenylamine (abbreviation: YGAPA), N,9-diphenyl-N-[4-(10-phenyl-9-anthryl)phenyl]-9H-carbazol-3-amine (abbreviation: PCAPA), N,9-diphenyl-N-{4-[4-(10-phenyl-9-anthryl)phenyl]phenyl}-9H-carbazol-3-amine (abbreviation: PCAPBA), N-(9,10-diphenyl-2-anthryl)-N,9-diphenyl-9H-carbazol-3-amine (abbreviation: 2PCAPA), 6,12-dimethoxy-5,11-diphenylchrysene, N,N,N′,N′,N″,N″,N′″,N′″-octaphenyldibenzo[g,p]chrysene-2,7,10,15-tetraamine (abbreviation: DBC1), CzPA, 7-[4-(10-phenyl-9-anthryl)phenyl]-7H-dibenzo[c,g]carbazole (abbreviation: cgDBCzPA), 6-[3-(9,10-diphenyl-2-anthryl)phenyl]-benzo[b]naphtho[1,2-d]furan (abbreviation: 2mBnfPPA), 9-phenyl-10-{4-(9-phenyl-9H-fluoren-9-yl)-biphenyl-4′-yl}anthracene (abbreviation: FLPPA), 9,10-bis(3,5-diphenylphenyl)anthracene (abbreviation: DPPA), 9,10-di(2-naphthyl)anthracene (abbreviation: DNA), 2-tert-butyl-9,10-di(2-naphthyl) anthracene (abbreviation: t-BuDNA), 9,9′-bianthryl(abbreviation: BANT), 9,9′-(stilbene-3,3′-diyl)diphenanthrene (abbreviation: DPNS), 9,9′-(stilbene-4,4′-diyl)diphenanthrene (abbreviation: DPNS2), 1,3,5-tri(1-pyrenyl)benzene (abbreviation: TPB3), 5,12-diphenyltetracene, and 5,12-bis(biphenyl-2-yl)tetracene.

In the case where the light-emitting substance is a phosphorescent material, as the organic compound used in combination with the light-emitting substance, an organic compound that has higher triplet excitation energy (energy difference between a ground state and a triplet excited state) than the light-emitting substance is selected.

In the case where a plurality of organic compounds (e.g., a first host material and a second host material (or an assist material)) are used in combination with the light-emitting substance in order to form an exciplex, the plurality of organic compounds are preferably mixed with a phosphorescent material (particularly an organometallic complex).

Such a structure makes it possible to efficiently obtain light emission utilizing ExTET (Exciplex-Triplet Energy Transfer), which is energy transfer from an exciplex to a light-emitting substance. Note that a combination of a plurality of organic compounds that easily forms an exciplex is preferable, and it is particularly preferable to combine a compound that easily accepts holes (a hole-transport material) and a compound that easily accepts electrons (an electron-transport material). As the hole-transport material and the electron-transport material, specifically, any of the materials described in this embodiment can be used. With this structure, high efficiency, low voltage, and a long lifetime of the light-emitting device can be achieved at the same time.

In the case where the light-emitting substance is a phosphorescent material, examples of the organic compounds that can be used in combination with the light-emitting substance include an aromatic amine, a carbazole derivative, a dibenzothiophene derivative, a dibenzofuran derivative, a zinc-or aluminum-based metal complex, an oxadiazole derivative, a triazole derivative, a benzimidazole derivative, a quinoxaline derivative, a dibenzoquinoxaline derivative, a pyrimidine derivative, a triazine derivative, a pyridine derivative, a bipyridine derivative, and a phenanthroline derivative.

Among the above-described compounds, specific examples of the aromatic amine, (a compound having an aromatic amine skeleton), the carbazole derivative, the dibenzothiophene derivative (thiophene derivative), and the dibenzofuran derivative (furan derivative), which are organic compounds having a high hole-transport property, are the same as the compounds given above as specific examples of the hole-transport material.

Specific examples of the zinc-and aluminum-based metal complexes, which are organic compounds having a high electron-transport property, include metal complexes having a quinoline skeleton or a benzoquinoline skeleton, such as tris(8-quinolinolato)aluminum(III) (abbreviation: Alq), tris(4-methyl-8-quinolinolato)aluminum(III) (abbreviation: Almq₃), bis(10-hydroxybenzo[h]quinolinato)beryllium(II) (abbreviation: BeBq₂), bis(2-methyl-8-quinolinolato)(4-phenylphenolato)aluminum(III) (abbreviation: BAlq), and bis(8-quinolinolato)zinc(II) (abbreviation: Znq).

Alternatively, a metal complex having an oxazole-based or thiazole-based ligand, such as bis[2-(2-benzoxazolyl)phenolato]zinc(II) (abbreviation: ZnPBO) or bis[2-(2-benzothiazolyl)phenolato]zinc(II) (abbreviation: ZnBTZ), or the like can also be used.

Specific examples of the oxadiazole derivative, the triazole derivative, the benzimidazole derivative, the benzimidazole derivative, the benzimidazole derivative, the quinoxaline derivative, the dibenzoquinoxaline derivative, and the phenanthroline derivative, which are organic compounds having a high electron-transport property, include 2-(4-biphenylyl)-5-(4-tert-butylphenyl)-1,3,4-oxadiazole (abbreviation: PBD), 1,3-bis[5-(p-tert-butylphenyl)-1,3,4-oxadiazol-2-yl]benzene (abbreviation: OXD-7), 9-[4-(5-phenyl-1,3,4-oxadiazol-2-yl)phenyl]-9H-carbazole (abbreviation: CO11), 3-(4-biphenylyl)-4-phenyl-5-(4-tert-butylphenyl)-1,2,4-triazole (abbreviation: TAZ), 3-(4-tert-butylphenyl)-4-(4-ethylphenyl)-5-(4-biphenylyl)-1,2,4-triazole (abbreviation: p-EtTAZ), 2,2′,2″-(1,3,5-benzenetriyl)tris(1-phenyl-1H-benzimidazole)2-[3-(dibenzothiophen-4-yl)phenyl]-1-phenyl-1H-benzimidazole (abbreviation: TPBI), (abbreviation: mDBTBIm-II), 4,4′-bis(5-methylbenzoxazol-2-yl)stilbene (abbreviation: BzOs), bathophenanthroline (abbreviation: Bphen), bathocuproine (abbreviation: BCP), 2,9-bis(naphthalen-2-yl)-4,7-diphenyl-1,10-phenanthroline (abbreviation: NBphen), 2-[3-(dibenzothiophen-4-yl)phenyl]dibenzo[f,h]quinoxaline (abbreviation: 2mDBTPDBq-II), 2-[3′-(dibenzothiophen-4-yl)biphenyl-3-yl]dibenzo[f,h]quinoxaline (abbreviation: 2mDBTBPDBq-II), 2-[3′-(9H-carbazol-9-yl)biphenyl-3-yl]dibenzo[f,h]quinoxaline (abbreviation: 2mCzBPDBq), 2-[4-(3,6-diphenyl-9H-carbazol-9-yl)phenyl]dibenzo[f,h]quinoxaline (abbreviation: 2CzPDBq-III), 7-[3-(dibenzothiophen-4-yl)phenyl]dibenzo[f,h]quinoxaline (abbreviation: 7mDBTPDBq-II), and 6-[3-(dibenzothiophen-4-yl)phenyl]dibenzo[f,h]quinoxaline (abbreviation: 6mDBTPDBq-II).

Specific examples of a heterocyclic compound having a diazine skeleton, a heterocyclic compound having a triazine skeleton, and a heterocyclic compound having a pyridine skeleton, which are organic compounds having a high electron-transport property, include 4,6-bis[3-(phenanthren-9-yl)phenyl]pyrimidine (abbreviation: 4,6mPnP2Pm), 4,6-bis[3-(4-dibenzothienyl)phenyl]pyrimidine (abbreviation: 4,6mDBTP2Pm-II), 4,6-bis[3-(9H-carbazol-9-yl)phenyl]pyrimidine (abbreviation: 4,6mCzP2Pm), 2-{4-[3-(N-phenyl-9H-carbazol-3-yl)-9H-carbazol-9-yl]phenyl}-4,6-diphenyl-1,3,5-triazine (abbreviation: PCCzPTzn), 9-[3-(4,6-diphenyl-1,3,5-triazin-2-yl)phenyl]-9′-phenyl-2,3′-bi-9H-carbazole (abbreviation: mPCCzPTzn-02), 3,5-bis[3-(9H-carbazol-9-yl)phenyl]pyridine (abbreviation: 35DCzPPy), and 1,3,5-tri[3-(3-pyridyl)phenyl]benzene (abbreviation: TmPyPB).

As the organic compound having a high electron-transport property, a high molecular compound such as poly (2,5-pyridinediyl) (abbreviation: PPy), poly[(9,9-dihexylfluorene-2,7-diyl)-co-(pyridine-3,5-diyl)] (abbreviation: PF-Py), or poly[(9,9-dioctylfluorene-2,7-diyl)-co-(2,2′-bipyridine-6,6′-diyl)] (abbreviation: PF-BPy) can also be used.

The TADF material is a material that can up-convert a triplet excited state into a singlet excited state (reverse intersystem crossing) using a little thermal energy and efficiently exhibits light emission (fluorescence) from the singlet excited state. Thermally activated delayed fluorescence is efficiently obtained under the condition where the difference in energy between the triplet excited level and the singlet excited level is greater than or equal to 0 eV and less than or equal to 0.2 eV, preferably greater than or equal to 0 eV and less than or equal to 0.1 eV. Delayed fluorescence by the TADF material refers to light emission having a spectrum similar to that of normal fluorescence and an extremely long lifetime. The lifetime is 10⁻⁶ seconds or longer, preferably 10⁻³ seconds or longer.

Examples of the TADF material include fullerene, a derivative thereof, an acridine derivative such as proflavine, and eosin. Other examples include a metal-containing porphyrin such as a porphyrin containing magnesium (Mg), zinc (Zn), cadmium (Cd), tin (Sn), platinum (Pt), indium (In), or palladium (Pd). Examples of the metal-containing porphyrin include a protoporphyrin-tin fluoride complex (abbreviation: SnF₂(Proto IX)), a mesoporphyrin-tin fluoride complex (abbreviation: SnF₂(Meso IX)), a hematoporphyrin-tin fluoride complex (abbreviation: SnF₂(Hemato IX)), a coproporphyrin tetramethyl ester-tin fluoride complex (abbreviation: SnF₂(Copro III-4Me)), an octaethylporphyrin-tin fluoride complex (abbreviation: SnF₂(OEP)), an etioporphyrin-tin fluoride complex (abbreviation: SnF₂(Etio I)), and an octaethylporphyrin-platinum chloride complex (abbreviation: PtCl₂OEP).

Alternatively, it is possible to use a heterocyclic compound having a π-electron rich heteroaromatic ring and a x-electron deficient heteroaromatic ring, such as 2-(biphenyl-4-yl)-4,6-bis(12-phenylindolo[2,3-a]carbazol-11-yl)-1,3,5-triazine (abbreviation: PIC-TRZ), PCCzPTzn, 2-[4-(10H-phenoxazin-10-yl)phenyl]-4,6-diphenyl-1,3,5-triazine (abbreviation: PXZ-TRZ), 3-[4-(5-phenyl-5,10-dihydrophenazin-10-yl)phenyl]-4,5-diphenyl-1,2,4-triazole (abbreviation: PPZ-3TPT), 3-(9,9-dimethyl-9H-acridin-10-yl)-9H-xanthen-9-one (abbreviation: ACRXTN), bis[4-(9,9-dimethyl-9, 10-dihydroacridine)phenyl]sulfone (abbreviation: DMAC-DPS), or 10-phenyl-10H,10′H-spiro[acridin-9,9′-anthracen]-10′-one (abbreviation: ACRSA). Note that a substance in which a π-electron rich heteroaromatic ring is directly bonded to a π-electron deficient heteroaromatic ring is particularly preferable because both the donor property of the π-electron rich heteroaromatic ring and the acceptor property of the x-electron deficient heteroaromatic ring are improved and the energy difference between the singlet excited state and the triplet excited state becomes small.

Note that the TADF material can also be used in combination with another organic compound. In particular, the TADF material can be used in combination with the host material, the hole-transport material, and the electron-transport material described above.

Furthermore, when used in combination with a low molecular material or a high molecular material, the above materials can be used to form the light-emitting layer 113. For the deposition, a known method (e.g., an evaporation method, a coating method, or a printing method) can be used as appropriate.

In the light-emitting device illustrated in FIG. 1C, the electron-transport layer 114 is formed over the light-emitting layer 113.

### Electron-Transport Layer

The electron-transport layer 114 is a layer that transports electrons, which are injected from the second electrode 102 by the electron-injection layer 115, to the light-emitting layer 113. Note that the electron-transport layer 114 is a layer containing an electron-transport material. As the electron-transport material used in the electron-transport layer 114, a substance having an electron mobility of greater than or equal to 1×10⁻⁶ cm²/Vs is preferable. Note that other substances can also be used as long as they have a property of transporting more electrons than holes.

As the electron-transport material, it is possible to use a material having a high electron-transport property, such as a metal complex having a quinoline skeleton, a metal complex having a benzoquinoline skeleton, a metal complex having an oxazole skeleton, a metal complex having a thiazole skeleton, an oxadiazole derivative, a triazole derivative, an imidazole derivative, an oxazole derivative, a thiazole derivative, a phenanthroline derivative, a quinoline derivative having a quinoline ligand, a benzoquinoline derivative, a quinoxaline derivative, a dibenzoquinoxaline derivative, a pyridine derivative, a bipyridine derivative, a pyrimidine derivative, or a T-electron deficient heteroaromatic compound such as a nitrogen-containing heteroaromatic compound.

As specific examples of the electron-transport material, the above-described materials can be used.

Next, in the light-emitting device illustrated in FIG. 1C, the electron-injection layer 115 is formed over the electron-transport layer 114 by a vacuum evaporation method.

### Electron-Injection Layer

The electron-injection layer 115 is a layer that contains a substance having a high electron-injection property. For the electron-injection layer 115, an alkali metal, an alkaline earth metal, or a compound thereof such as lithium fluoride (LiF), cesium fluoride (CsF), calcium fluoride (CaF₂), or lithium oxide (LiOₓ) can be used. A rare earth metal compound like erbium fluoride (ErF₃) can also be used. In addition, an electride may be used for the electron-injection layer 115. An example of the electride is a substance in which electrons are added at high concentration to calcium oxide-aluminum oxide. Any of the above-described substances for forming the electron-transport layer 114 can also be used.

Alternatively, for the electron-injection layer 115, a composite material containing an electron-transport material and a donor material (an electron-donating material) may be used. Such a composite material is excellent in an electron-injection property and an electron-transport property because electrons are generated in the organic compound by the electron donor. The organic compound here is preferably a material excellent in transporting the generated electrons; specifically, for example, the above-described electron-transport materials used in the electron-transport layer 114 (e.g., a metal complex or a heteroaromatic compound) can be used. As the electron donor, a substance showing an electron-donating property with respect to an organic compound is used. Specifically, an alkali metal, an alkaline earth metal, and a rare earth metal are preferable, and lithium, cesium, magnesium, calcium, erbium, ytterbium, and the like are given. In addition, an alkali metal oxide and an alkaline earth metal oxide are preferable, and lithium oxide, calcium oxide, barium oxide, and the like are given. Alternatively, a Lewis base such as magnesium oxide can be used. Further alternatively, an organic compound such as tetrathiafulvalene (abbreviation: TTF) can be used.

### Charge-Generation Layer

In the light-emitting device illustrated in FIG. 1B, the charge-generation layer 104 has a function of injecting electrons into the EL layer 103a and injecting holes into the EL layer 103b when voltage is applied between the first electrode 101 (the anode) and the second electrode 102 (the cathode).

The charge-generation layer 104 may contain a hole-transport material and an acceptor material (an electron-accepting material) or may contain an electron-transport material and a donor material. Forming the charge-generation layer 104 with such a structure can suppress an increase in the driving voltage that would be caused by stacking EL layers.

As the hole-transport material, the acceptor material, the electron-transport material, and the donor material, the above-described materials can be used.

For fabrication of the light-emitting device in this embodiment, a vacuum process such as an evaporation method or a solution process such as a spin coating method or an ink-jet method can be used. In the case of using an evaporation method, a physical vapor deposition method (PVD method) such as a sputtering method, an ion plating method, an ion beam evaporation method, a molecular beam evaporation method, or a vacuum evaporation method, a chemical vapor deposition method (CVD method), or the like can be used. Specifically, the functional layers (the hole-injection layer, the hole-transport layer, the light-emitting layer, the electron-transport layer, and the electron-injection layer) included in the EL layer and the charge-generation layer can be formed by an evaporation method (e.g., a vacuum evaporation method), a coating method (e.g., a dip coating method, a die coating method, a bar coating method, a spin coating method, or a spray coating method), a printing method (e.g., an ink-jet method, screen printing (stencil), offset printing (planography), flexography (relief printing), gravure printing, or micro-contact printing), or the like.

Materials of the functional layers included in the EL layer 103 and the charge-generation layer are not limited to the above-described corresponding materials. For example, as the materials of the functional layers, a high molecular compound (e.g., an oligomer, a dendrimer, and a polymer), a middle molecular compound (a compound between a low molecular compound and a high molecular compound with a molecular weight of 400 to 4000), or an inorganic compound (e.g., a quantum dot material) may be used. As the quantum dot material, a colloidal quantum dot material, an alloyed quantum dot material, a core-shell quantum dot material, a core quantum dot material, or the like can be used.

### Structure Example 1 of Light-Emitting Apparatus

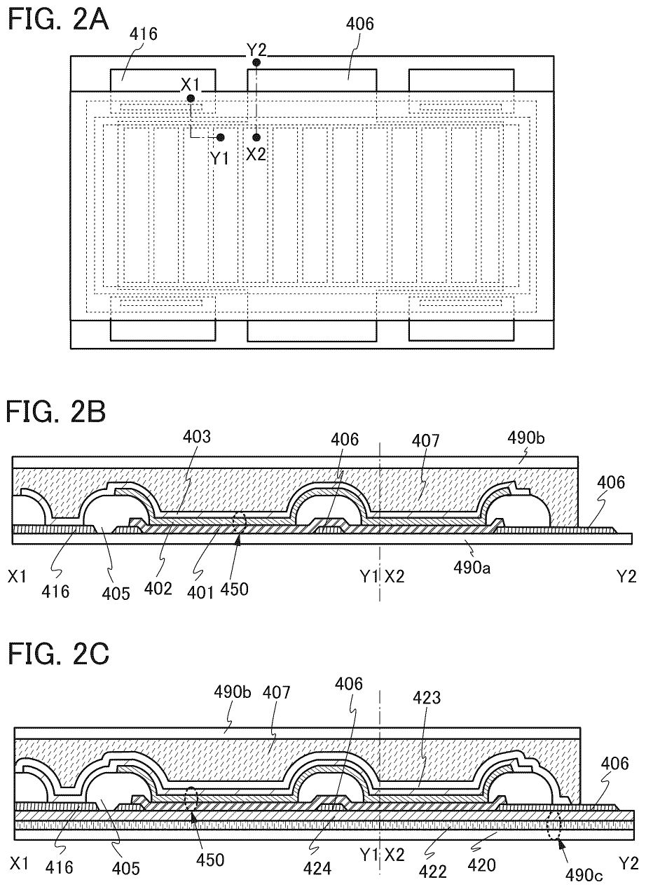

FIG. 2A is a top view of a light-emitting apparatus, and FIG. 2B and FIG. 2C are cross-sectional views along the dashed-dotted lines X1-Y1 and X2-Y2 in FIG. 2A. The light-emitting apparatus illustrated in FIG. 2A to FIG. 2C can be used as a lighting apparatus, for example. The light-emitting apparatus can have a bottom-emission, top-emission, or dual-emission structure.

The light-emitting apparatus illustrated in FIG. 2B includes a substrate 490a, a substrate 490b, a conductive layer 406, a conductive layer 416, an insulating layer 405, an organic EL device 450 (a first electrode 401, an EL layer 402, and a second electrode 403), and an adhesive layer 407. The organic EL device 450 can also be referred to as a light-emitting element, an organic EL element, a light-emitting device, or the like. In the EL layer 402, a light-emitting layer preferably contains any of the organometallic complexes described in Embodiment 1 as a light-emitting organic compound.

The organic EL device 450 includes the first electrode 401 over the substrate 490a, the EL layer 402 over the first electrode 401, and the second electrode 403 over the EL layer 402. The organic EL device 450 is sealed by the substrate 490a, the adhesive layer 407, and the substrate 490b.

End portions of the first electrode 401, the conductive layer 406, and the conductive layer 416 are covered with the insulating layer 405. The conductive layer 406 is electrically connected to the first electrode 401, and the conductive layer 416 is electrically connected to the second electrode 403. The conductive layer 406 covered with the insulating layer 405 with the first electrode 401 positioned therebetween functions as an auxiliary wiring and is electrically connected to the first electrode 401. It is preferable that the auxiliary wiring electrically connected to the electrode of the organic EL device 450 be provided, in which case a voltage drop due to the resistance of the electrode can be inhibited. The conductive layer 406 may be provided over the first electrode 401. An auxiliary wiring that is electrically connected to the second electrode 403 may be provided, for example, over the insulating layer 405.

For each of the substrate 490a and the substrate 490b, glass, quartz, ceramic, sapphire, an organic resin, or the like can be used. When a flexible material is used for the substrate 490a and the substrate 490b, the flexibility of the display apparatus can be increased.

A light-emitting surface of the light-emitting apparatus may be provided with a light extraction structure for increasing the light extraction efficiency, an antistatic film preventing the attachment of a foreign substance, a water repellent film suppressing the attachment of stain, a hard coat film suppressing generation of a scratch in use, an impact absorption layer, or the like.

Examples of an insulating material that can be used for the insulating layer 405 include a resin such as an acrylic resin and an epoxy resin, and an inorganic insulating material such as silicon oxide, silicon oxynitride, silicon nitride oxide, silicon nitride, and aluminum oxide.

For the adhesive layer 407, a variety of curable adhesives, e.g., a photocurable adhesive such as an ultraviolet curable adhesive, a reactive curable adhesive, a thermosetting adhesive, and an anaerobic adhesive can be used. Examples of these adhesives include an epoxy resin, an acrylic resin, a silicone resin, a phenol resin, a polyimide resin, an imide resin, a PVC (polyvinyl chloride) resin, a PVB (polyvinyl butyral) resin, and an EVA (ethylene vinyl acetate) resin. In particular, a material with low moisture permeability, such as an epoxy resin, is preferred. Alternatively, a two-component resin may be used. Alternatively, an adhesive sheet or the like may be used.

The light-emitting apparatus illustrated in FIG. 2C includes a barrier layer 490c, the conductive layer 406, the conductive layer 416, the insulating layer 405, the organic EL device 450, the adhesive layer 407, a barrier layer 423, and the substrate 490b.

The barrier layer 490c illustrated in FIG. 2C includes a substrate 420, an adhesive layer 422, and an insulating layer 424 having a high barrier property.

In the light-emitting apparatus illustrated in FIG. 2C, the organic EL device 450 is provided between the insulating layer 424 having a high barrier property and the barrier layer 423. Thus, even when resin films with relatively low water resistance or the like are used as the substrate 420 and the substrate 490b, a reduction in lifetime due to entry of impurities such as water into the organic EL device can be suppressed.

For each of the substrate 420 and the substrate 490b, for example, a polyester resin such as polyethylene terephthalate (PET) or polyethylene naphthalate (PEN), a polyacrylonitrile resin, an acrylic resin, a polyimide resin, a polymethyl methacrylate resin, a polycarbonate (PC) resin, a polyethersulfone (PES) resin, a polyamide resin (e.g., nylon or aramid), a polysiloxane resin, a cycloolefin resin, a polystyrene resin, a polyamide-imide resin, a polyurethane resin, a polyvinyl chloride resin, a polyvinylidene chloride resin, a polypropylene resin, a polytetrafluoroethylene (PTFE) resin, an ABS resin, cellulose nanofiber, or the like can be used. Glass that is thin enough to have flexibility may be used for the substrate 420 and the substrate 490b.

An inorganic insulating film is preferably used as the insulating layer 424 having a high barrier property. As the inorganic insulating film, a silicon nitride film, a silicon oxynitride film, a silicon oxide film, a silicon nitride oxide film, an aluminum oxide film, or an aluminum nitride film can be used, for example. A hafnium oxide film, an yttrium oxide film, a zirconium oxide film, a gallium oxide film, a tantalum oxide film, a magnesium oxide film, a lanthanum oxide film, a cerium oxide film, a neodymium oxide film, or the like may also be used. A stack including two or more of the above insulating films may also be used.

The barrier layer 423 preferably includes at least a single-layer inorganic film. For example, the barrier layer 423 can have a single-layer structure of an inorganic film or a stacked-layer structure of an inorganic film and an organic film. As the inorganic film, the above-described inorganic insulating film is preferable. An example of the stacked-layer structure is a structure in which a silicon oxynitride film, a silicon oxide film, an organic film, a silicon oxide film, and a silicon nitride film are formed in this order. When the protective layer has a stacked-layer structure of an inorganic film and an organic film, entry of impurities that can enter the organic EL device 450 (typically, hydrogen, water, and the like) can be suitably suppressed.

The insulating layer 424 having a high barrier property and the organic EL device 450 can be directly formed on the substrate 420 having flexibility. In that case, the adhesive layer 422 is not necessary. Alternatively, the insulating layer 424 and the organic EL device 450 can be formed over a rigid substrate with a separation layer provided therebetween and then transferred to the substrate 420. For example, the insulating layer 424 and the organic EL device 450 may be transferred to the substrate 420 in the following manner: the insulating layer 424 and the organic EL device 450 are separated from the rigid substrate by applying heat, force, laser light, or the like to the separation layer, and then the insulating layer 424 and the organic EL device 450 are bonded to the substrate 420 with the use of the adhesive layer 422. For the separation layer, a stacked-layer structure of inorganic films including a tungsten film and a silicon oxide film, or an organic resin film of polyimide or the like can be used, for example. In the case where a rigid substrate is used, the insulating layer 424 can be formed at high temperature as compared to the case where a resin substrate or the like is used; thus, the insulating layer 424 can have high density and an excellent barrier property.

### Structure Example 2 of Light-Emitting Apparatus

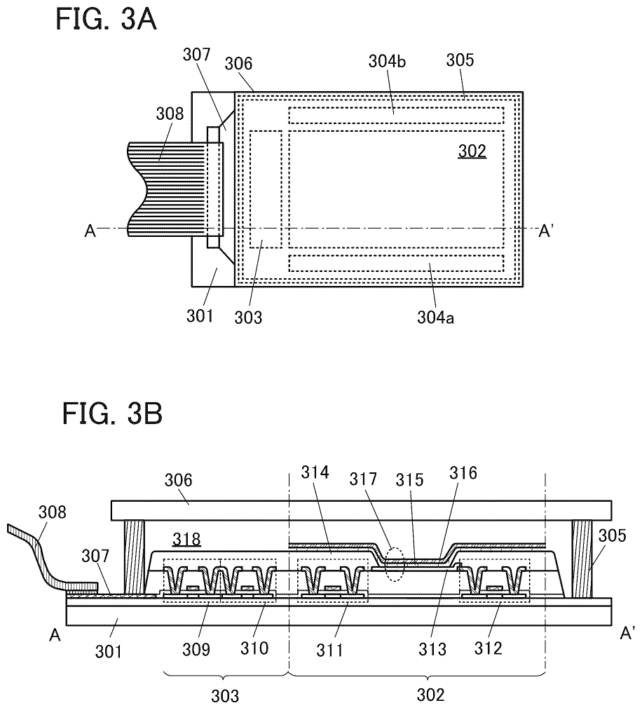

The light-emitting apparatus of one embodiment of the present invention can be of passive matrix type or active matrix type. An active-matrix light-emitting apparatus will be described with reference to FIG. 3.

FIG. 3A is a top view of the light-emitting apparatus. FIG. 3B is a cross-sectional view along the dashed-dotted line A-A′ in FIG. 3A.

The active-matrix light-emitting apparatus illustrated in FIG. 3A and FIG. 3B includes a pixel portion 302, a circuit portion 303, a circuit portion 304a, and a circuit portion 304b.

Each of the circuit portion 303, the circuit portion 304a, and the circuit portion 304b can function as a scan line driver circuit (a gate driver) or a signal line driver circuit (a source driver), or may be a circuit that electrically connects the pixel portion 302 to an external gate driver or source driver.

A lead wiring 307 is provided over a first substrate 301. The lead wiring 307 is electrically connected to an FPC 308 that is an external input terminal. The FPC 308 transmits signals (e.g., a video signal, a clock signal, a start signal, and a reset signal) and a potential from the outside to the circuit portion 303, the circuit portion 304a, and the circuit portion 304b. The FPC 308 may be provided with a printed wiring board (PWB). The structure illustrated in FIG. 3A and FIG. 3B can also be referred to as a light-emitting module including a light-emitting device (or a light-emitting apparatus) and an FPC.

The pixel portion 302 includes a plurality of pixels each including an organic EL device 317, a transistor 311, and a transistor 312. The transistor 312 is electrically connected to a first electrode 313 included in the organic EL device 317. The transistor 311 functions as a switching transistor. The transistor 312 functions as a current control transistor. Note that the number of transistors included in each pixel is not particularly limited and can be set appropriately as needed.

The circuit portion 303 includes a plurality of transistors, such as a transistor 309 and a transistor 310. The circuit portion 303 may be configured with a circuit including transistors having the same conductivity type (either n-channel transistors or p-channel transistors), or may be configured with a CMOS circuit including an n-channel transistor and a p-channel transistor. Furthermore, a driver circuit may be provided outside.

There is no particular limitation on the structure of the transistor included in the light-emitting apparatus of this embodiment. For example, a planar transistor, a staggered transistor, or an inverted staggered transistor can be used. A top-gate or a bottom-gate transistor structure may be used. Alternatively, gates may be provided above and below a semiconductor layer where a channel is formed.

There is no particular limitation on the crystallinity of a semiconductor material used for the transistor, and an amorphous semiconductor or a semiconductor having crystallinity (a microcrystalline semiconductor, a polycrystalline semiconductor, a single crystal semiconductor, or a semiconductor partly including crystal regions) can be used. A semiconductor having crystallinity is preferably used, in which case deterioration of the transistor characteristics can be suppressed.

It is preferable that the semiconductor layer of the transistor contain a metal oxide (also referred to as an oxide semiconductor). Alternatively, the semiconductor layer of the transistor may contain silicon. Examples of silicon include amorphous silicon and crystalline silicon (e.g., low-temperature polysilicon and single crystal silicon).

The semiconductor layer preferably contains indium, M (M is one or more kinds selected from gallium, aluminum, silicon, boron, yttrium, tin, copper, vanadium, beryllium, titanium, iron, nickel, germanium, zirconium, molybdenum, lanthanum, cerium, neodymium, hafnium, tantalum, tungsten, and magnesium), and zinc, for example. Specifically, M is preferably one or more kinds selected from aluminum, gallium, yttrium, and tin.

It is particularly preferable to use an oxide containing indium (In), gallium (Ga), and zinc (Zn) (also referred to as IGZO) for the semiconductor layer.

In the case where the semiconductor layer is an In-M-Zn oxide, a sputtering target used for depositing the In-M-Zn oxide preferably has the atomic proportion of In higher than or equal to the atomic proportion of M. Examples of the atomic ratio of the metal elements in such a sputtering target include In:M:Zn=1:1:1, In:M:Zn=1:1:1.2, In:M:Zn=2:1:3, In:M:Zn=3:1:2, In:M:Zn=4:2:3, In:M:Zn=4:2:4.1, In:M:Zn=5:1:6, In:M:Zn=5:1:7, In:M:Zn=5:1:8, In:M:Zn=6:1:6, and In:M:Zn=5:2:5.

The transistors included in the circuit portion 303, the circuit portion 304a, and the circuit portion 304b and the transistors included in the pixel portion 302 may have the same structure or different structures. A plurality of transistors included in the circuit portion 303, the circuit portion 304a, and the circuit portion 304b may have the same structure or two or more kinds of structures. Similarly, a plurality of transistors included in the pixel portion 302 may have the same structure or two or more kinds of structures.

An end portion of the first electrode 313 is covered with an insulating layer 314. For the insulating layer 314, an organic compound such as a negative photosensitive resin or a positive photosensitive resin (acrylic resin), or an inorganic compound such as silicon oxide, silicon oxynitride, or silicon nitride can be used. An upper end portion or a lower end portion of the insulating layer 314 preferably has a curved surface with curvature. In that case, favorable coverage with a film formed over the insulating layer 314 can be obtained.

An EL layer 315 is provided over the first electrode 313, and a second electrode 316 is provided over the EL layer 315. The EL layer 315 includes a light-emitting layer, a hole-injection layer, a hole-transport layer, an electron-transport layer, an electron-injection layer, a charge-generation layer, and the like. In the EL layer 315, the light-emitting layer preferably contains any of the organometallic complexes described in Embodiment 1 as a light-emitting organic compound.

The plurality of transistors and the plurality of organic EL devices 317 are sealed by the first substrate 301, a second substrate 306, and a sealant 305. A space 318 surrounded by the first substrate 301, the second substrate 306, and the sealant 305 may be filled with an inert gas (e.g., nitrogen or argon) or an organic substance (including the sealant 305).

An epoxy resin or glass frit can be used for the sealant 305. A material that transmits moisture and oxygen as little as possible is preferably used for the sealant 305. In the case where glass frit is used for the sealant, the first substrate 301 and the second substrate 306 are preferably glass substrates in terms of adhesion.

This embodiment can be combined with the other embodiments as appropriate.

### Embodiment 3

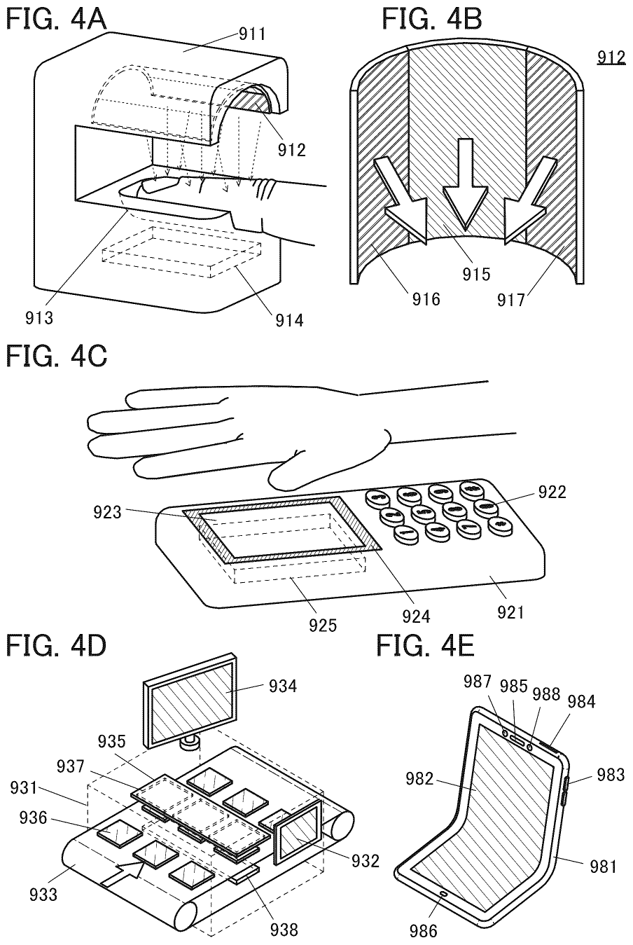

In this embodiment, electronic apparatuses in which the light-emitting device of one embodiment of the present invention can be used will be described with reference to FIG. 4.

FIG. 4A illustrates a biometric authentication apparatus for sensing a finger vein which includes a housing 911, a light source 912, a sensing stage 913, and the like. By putting a finger on the sensing stage 913, an image of a vein pattern can be captured. The light source 912 that emits near-infrared light is provided above the sensing stage 913, and an imaging device 914 is provided under the sensing stage 913. The sensing stage 913 is formed of a material that transmits near-infrared light, and near-infrared light that is emitted from the light source 912 and passes through the finger can be captured by the imaging device 914. Note that an optical system may be provided between the sensing stage 913 and the imaging device 914. The structure of the apparatus described above can also be applied to a biometric authentication apparatus for sensing a palm vein.

The light-emitting device of one embodiment of the present invention can be used for the light source 912. The light-emitting device of one embodiment of the present invention can be provided to be curved and can emit light uniformly toward a target. In particular, the light-emitting device preferably emits near-infrared light with the maximum peak intensity at a wavelength from 760 nm to 900 nm. An image is formed from received light that has passed through the finger, palm, or the like, whereby the position of the vein can be detected. This action can be utilized for biometric authentication. A combination with a global shutter system enables highly accurate sensing even for a moving target.

The light source 912 can include a plurality of light-emitting portions, such as light-emitting portions 915, 916, and 917 illustrated in FIG. 4B. The light-emitting portions 915, 916, and 917 may emit light having different wavelengths, or can emit light at different timings. Thus, by changing wavelengths and angles of light to be delivered, different images can be taken successively; hence, high level of security can be achieved using a plurality of images for the authentication.

FIG. 4C illustrates a biometric authentication apparatus for sensing a palm vein which includes a housing 921, operation buttons 922, a sensing portion 923, a light source 924 that emits near-infrared light, and the like. By holding a hand over the sensing portion 923, a palm vein pattern can be recognized. Furthermore, a security code or the like can be input with the operation buttons. The light source 924 is provided around the sensing portion 923 and irradiates a target (a hand) with light. Then, light reflected by the target enters the sensing portion 923. The light-emitting device of one embodiment of the present invention can be used for the light source 924. An imaging device 925 is provided directly under the sensing portion 923 and can capture an image of the target (an image of the whole hand). Note that an optical system may be provided between the sensing portion 923 and the imaging device 925. The structure of the apparatus described above can also be applied to a biometric authentication apparatus for sensing a finger vein.

FIG. 4D illustrates a non-destructive testing apparatus that includes a housing 931, an operation panel 932, a transport mechanism 933, a monitor 934, a sensing unit 935, a light source 938 that emits near-infrared light, and the like. The light-emitting device of one embodiment of the present invention can be used for the light source 938. Test specimens 936 are transported by the transport mechanism 933 to be located directly beneath the sensing unit 935. The test specimen 936 is irradiated with near-infrared light from the light source 938, and the light passing therethrough is captured by an imaging device 937 provided in the sensing unit 935. The captured image is displayed on the monitor 934. After that, the test specimens 936 are transported to the exit of the housing 931, and defective pieces are sorted and collected. Imaging with the use of near-infrared light enables non-destructive and high-speed sensing of defective elements inside the test specimen, such as defects and foreign substances.

FIG. 4E illustrates a mobile phone that includes a housing 981, a display portion 982, an operation button 983, an external connection port 984, a speaker 985, a microphone 986, a first camera 987, a second camera 988, and the like. The display portion 982 of the mobile phone includes a touch sensor. The housing 981 and the display portion 982 have flexibility. All operations including making a call and inputting text can be performed by touch on the display portion 982 with a finger, a stylus, or the like. The first camera 987 can take a visible light image, and the second camera 988 can take an infrared light image (a near-infrared light image). The mobile phone or the display portion 982 illustrated in FIG. 4E may include the light-emitting device of one embodiment of the present invention.

This embodiment can be combined with the other embodiments as appropriate.

### EXAMPLE 1

### Synthesis Example 1

In this example, a method for synthesizing an organometallic complex of one embodiment of the present invention will be described. In this example, the description is made on a method for synthesizing bis{4,6-dimethyl-2-[3-(3,5-dimethylphenyl)-2-benzo[g]quinoxalinyl-κN]phenyl-κC}(2,2,6,6-tetramethyl-3,5-heptanedionato-κ²O,O′)iridium(III) (abbreviation: [Ir(dmdpbq)₂(dpm)]) that is represented by Structural Formula (100) in Embodiment 1.

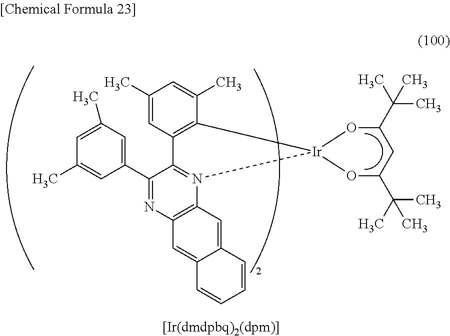

Step 1: Synthesis of 2,3-bis-(3,5-dimethylphenyl)-2-benzo[g]quinoxaline (Abbreviation: Hdmdpbq)

First, in Step 1, Hdmdpbq that is an organic compound of one embodiment of the present invention and is represented by Structural Formula (200) was synthesized. Into a three-necked flask equipped with a reflux pipe, 3.20 g of 3,3′,5,5′-tetramethylbenzyl, 1.97 g of 2,3-diaminonaphthalene, and 60 mL of ethanol were put, the air in the flask was replaced with nitrogen, and then the mixture was stirred at 90° C. for 7.5 hours. After a predetermined time elapsed, the solvent was distilled off. Then, purification by silica gel column chromatography using toluene as a developing solvent was performed, whereby the target substance was obtained (a yellow solid, yield: 3.73 g, percent yield: 79%). The synthesis scheme of Step 1 is shown in (a-1).

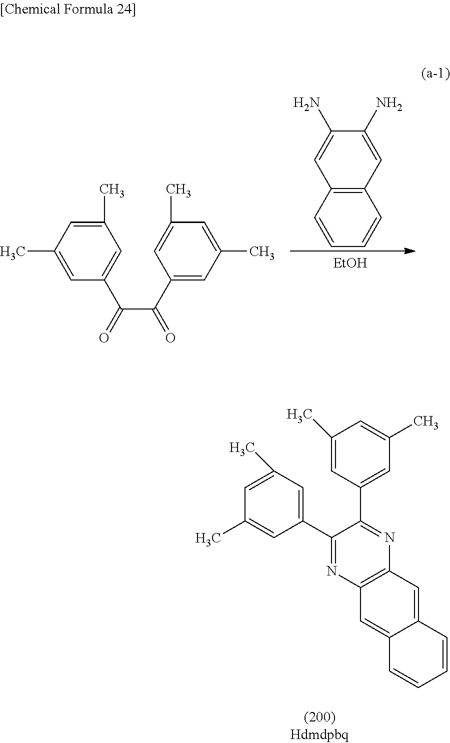

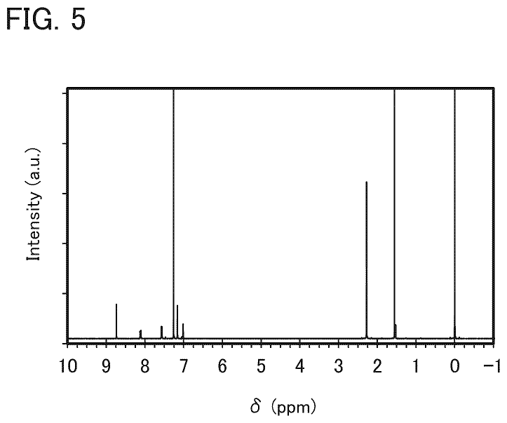

Analysis results by nuclear magnetic resonance spectroscopy (¹H-NMR) of the yellow solid obtained in Step 1 are shown below. FIG. 5 is the ¹H-NMR chart. These revealed that Hdmdpbq, which is represented by Structural Formula (200), was obtained in this example.

Given below is ¹H NMR data of the obtained substance.
¹H-NMR. δ (CD₂Cl₂): 2.28 (s, 12H), 7.01 (s, 2H), 7.16 (s, 4H), 7.56-7.58 (m, 2H), 8.11-8.13 (m, 2H), 8.74 (s, 2H).
   Step 2: Synthesis of di-μ-chloro-tetrakis{4,6-dimethyl-2-[3-(3,5-dimethylphenyl)-2-benzo[g]quinoxalinyl-κN] phenyl-κC}diiridium(III) (Abbreviation: [Ir(dmdpbq)₂Cl]₂)

Next, in Step 2, [Ir(dmdpbq)₂Cl]₂ that is a dinuclear complex of one embodiment of the present invention and is represented by Structural Formula (210) was synthesized. Into a recovery flask equipped with a reflux pipe, 15 mL of 2-ethoxyethanol, 5 mL of water, 1.81 g of Hdmdpbq obtained in Step 1, and 0.66 g of iridium chloride hydrate (IrCl₃.H₂O) (produced by Furuya Metal Co., Ltd.) were put, and the air in the flask was replaced with argon. Then, microwave irradiation (2.45 GHz, 100 W) was performed for 2 hours to cause reaction. After a predetermined time elapsed, the obtained residue was suction-filtered and washed with methanol, whereby the target substance was obtained (a black solid, yield: 1.76 g, percent yield: 81%). The synthesis scheme of Step 2 is shown in (a-2).

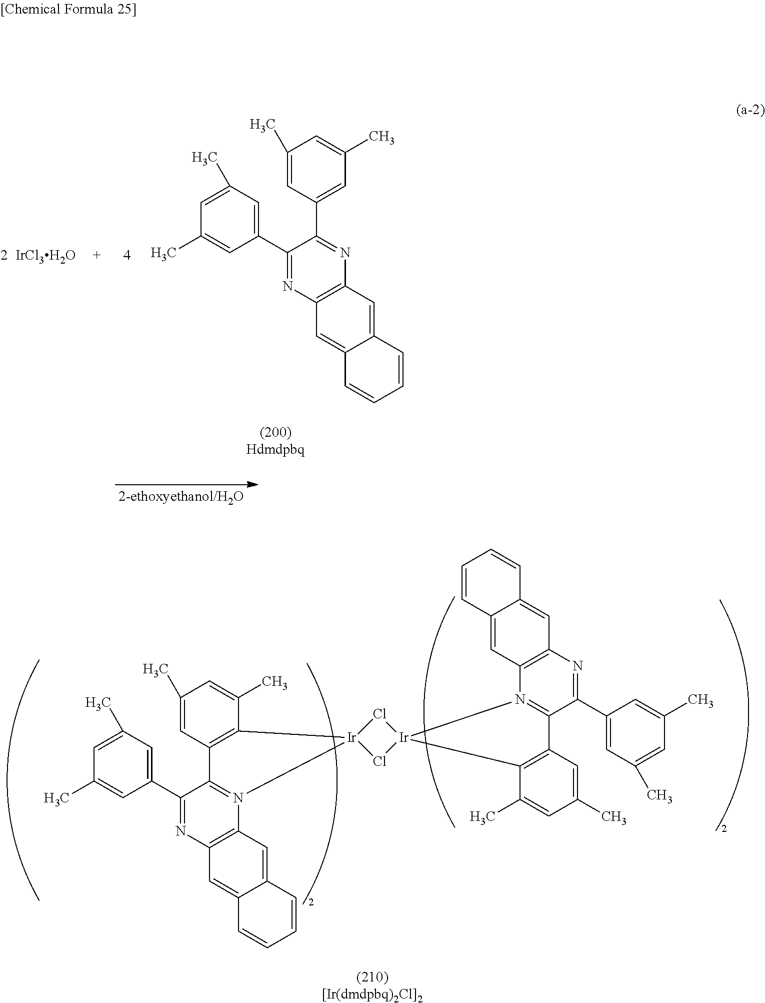

### Step 3: Synthesis of [Ir(dmdpbq)₂(dpm)]

Then, in Step 3, [Ir(dmdpbq)₂(dpm)], which is the organometallic complex of one embodiment of the present invention and is represented by Structural Formula (100), was synthesized. Into a recovery flask equipped with a reflux pipe, 20 mL of 2-ethoxyethanol, 1.75 g of [Ir(dmdpbq)₂Cl]₂ obtained in Step 2, 0.50 g of dipivaloylmethane (abbreviation: Hdpm), and 0.95 g of sodium carbonate were put, and the air in the flask was replaced with argon. Then, microwave irradiation (2.45 GHz, 100 W) was performed for 3 hours. The obtained residue was suction-filtered with methanol and then washed with water and methanol. The obtained solid was purified by silica gel column chromatography using dichloromethane as a developing solvent, and then recrystallization was performed with a mixed solvent of dichloromethane and methanol, whereby the target substance was obtained (a dark green solid, yield: 0.42 g, percent yield: 21%). With a train sublimation method, 0.41 g of the obtained dark green solid was purified by sublimation. The conditions of the sublimation purification were such that the dark green solid was heated under a pressure of 2.7 Pa at 300° C. while the argon gas flowed at a flow rate of 10.5 mL/min. After the sublimation purification, a dark green solid was obtained in a yield of 78%. The synthesis scheme of Step 3 is shown in (a-3).

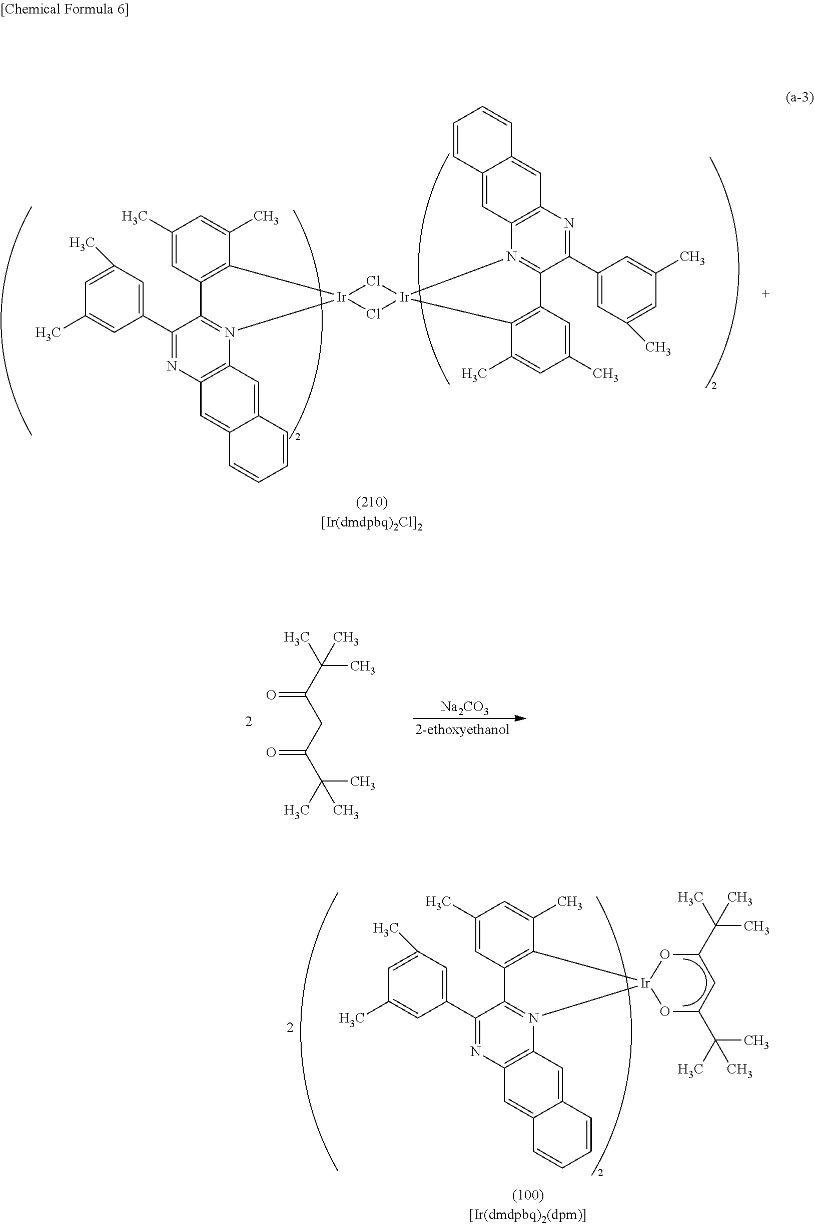

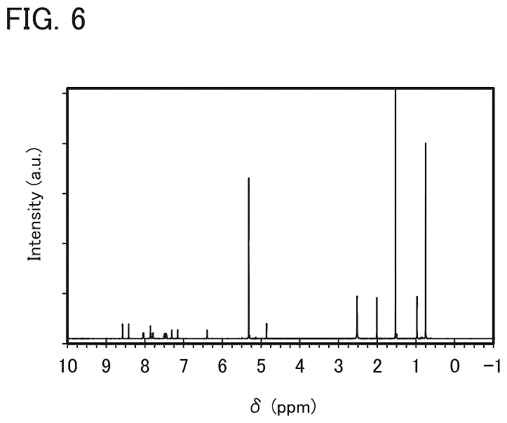

Results of analysis by nuclear magnetic resonance spectroscopy (¹H-NMR) of the dark green solid obtained in Step 3 are shown below. FIG. 6 shows the ¹H-NMR chart. These revealed that [Ir(dmdpbq)₂(dpm)] represented by Structural Formula (100) was obtained in this example.

Given below is ¹H NMR data of the obtained substance.
¹H-NMR. δ (CD₂Cl₂): 0.75 (s, 18H), 0.97 (s, 6H), 2.01 (s, 6H), 2.52 (s, 12H), 4.86 (s, 1H), 6.39 (s, 2H), 7.15 (s, 2H), 7.31 (s, 2H), 7.44-7.51 (m, 4H), 7.80 (d, 2H), 7.86 (s, 4H), 8.04 (d, 2H), 8.42 (s, 2H), 8.58 (s, 2H).

Next, an ultraviolet-visible absorption spectrum (hereinafter simply referred to as an “absorption spectrum”) and an emission spectrum of a dichloromethane solution of [Ir(dmdpbq)₂(dpm)] were measured.

The measurement of the absorption spectrum was conducted at room temperature, for which an ultraviolet-visible spectrophotometer (V550 type, manufactured by JASCO Corporation) was used and the dichloromethane solution (0.010 mmol/L) was put into a quartz cell. The measurement of the emission spectrum was conducted at room temperature, for which a fluorescence spectrophotometer (FS920, manufactured by Hamamatsu Photonics K.K.) was used and the deoxygenated dichloromethane solution (0.010 mmol/L) was put and hermetically sealed into a quartz cell in a nitrogen atmosphere.

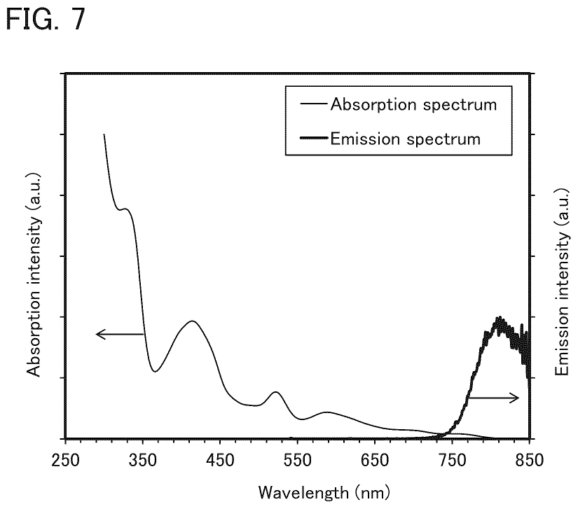

FIG. 7 shows measurement results of the obtained absorption spectrum and emission spectrum. The horizontal axis represents the wavelength, and the vertical axes represent the absorption intensity and the emission intensity. The thin solid line in FIG. 7 represents the absorption spectrum, and the thick solid line represents the emission spectrum. The absorption spectrum shown in FIG. 7 is the results obtained in such a way that the absorption spectrum measured by putting only dichloromethane in a quartz cell was subtracted from the absorption spectrum measured by putting the dichloromethane solution (0.010 mmol/L) in a quartz cell.

As shown in FIG. 7, [Ir(dmdpbq)₂(dpm)], which is the organometallic complex of one embodiment of the present invention, has an emission peak at 807 nm, and near-infrared light was observed from the dichloromethane solution.

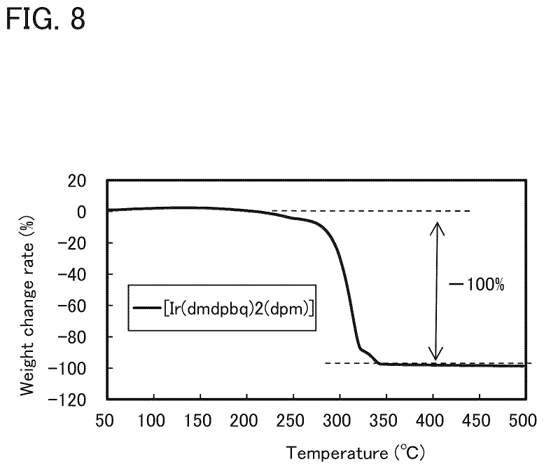

In addition, the weight loss percentage of the obtained [Ir(dmdpbq)₂(dpm)] was measured by a high-vacuum differential type differential thermal balance (TG-DTA 2410SA, manufactured by Bruker AXS K.K.). The temperature was increased at a rate of 10° C./min under a degree of vacuum of 10 Pa; as a result, as shown in FIG. 8, the weight loss percentage of [Ir(dmdpbq)₂(dpm)], which is the organometallic complex of one embodiment of the present invention, was 100% (corresponding to a decrease rate of −100% in FIG. 8), which demonstrates favorable sublimability.

As demonstrated above, [Ir(dmdpbq)₂(dpm)], which is the organometallic complex of one embodiment of the present invention, was synthesized in this example. In addition, [Ir(dmdpbq)₂(dpm)] was found to be capable of emitting near-infrared light and have favorable sublimability.

Note that [Ir(dmdpbq)₂(dpm)] is an example of the case where X in General Formula (G1) is an unsubstituted benzene ring. It is probable that X being a benzene ring can extend a π-conjugated system, deepen the LUMO level, and provide energetic stability, and thus emission of near-infrared light was obtained. Moreover, [Ir(dmdpbq)₂(dpm)] is an example of the case where all of R¹, R³, R⁶, and R⁸ in General Formula (G1) are methyl groups. It is probable that all of R¹, R³, R⁶, and R⁸ being methyl groups can suppress a decrease in sublimability even when X is a condensed ring (a benzene ring), and thus the organometallic complex that has favorable sublimability and emits near-infrared light was obtained.

### EXAMPLE 2

In this example, the results of fabricating a light-emitting device of one embodiment of the present invention will be described. Specifically, the description is made on a structure, a fabrication method, and characteristics of a light-emitting device 1 using bis{4,6-dimethyl-2-[3-(3,5-dimethylphenyl)-2-benzo[g]quinoxalinyl-κN]phenyl-κC}(2,2,6,6-tetramethyl-3,5-heptanedionato-κ²O,O′)iridium(III) abbreviation: [Ir(dmdpbq)₂(dpm)]) (Structural Formula (100)), which is described in Example 1, in a light-emitting layer.

The light-emitting device 1 fabricated in this example is an example of a light-emitting device in which the light-emitting layer contains a light-emitting organic compound and the maximum peak wavelength of light emitted from the light-emitting organic compound is greater than or equal to 760 nm and less than or equal to 900 nm. Moreover, the light-emitting device 1 is an example of a light-emitting device using an organometallic complex having a metal-carbon bond as the light-emitting organic compound.

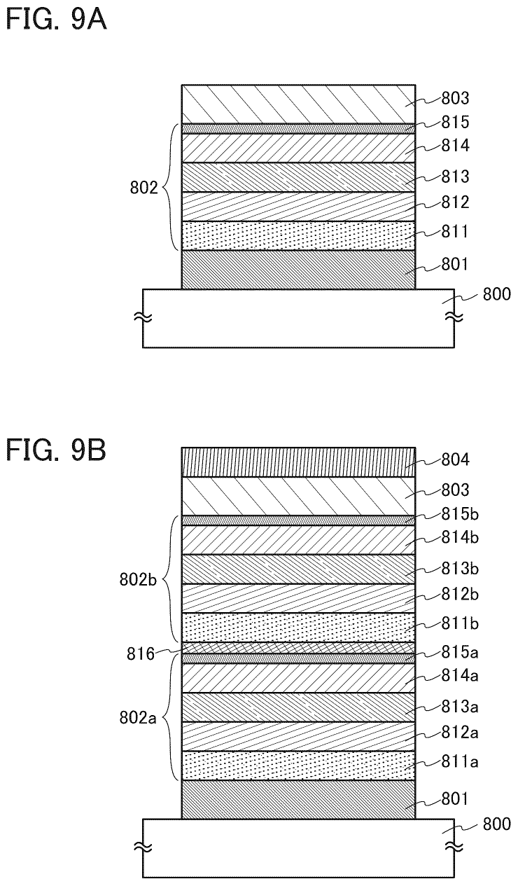

FIG. 9A illustrates the structure of the light-emitting device 1 used in this example, and Table 1 shows specific components. The chemical formulae of the materials used in this example are shown below.

TABLE 1

| | | | Hole- | Light- | | | Electron- | |
|---|---|---|---|---|---|---|---|---|
| | First | Hole-injection | transport | emitting | | | injection | Second |
| | electrode | layer | layer | layer | Electron-transport layer | | layer | electrode |
| | 801 | 811 | 812 | 813 | 814 | | 815 | 803 |
| Light- | ITSO | DBT3P-II:MoOx | BPAFLP | * | 2mDBTBPDBq-II | NBphen | LiF | Al |
| emitting | (110 nm) | (2:1 60 nm) | (20 nm) | | (20 nm) | (70 nm) | (1 nm) | (200 nm) |
| device 1 | | | | | | | | |

*2mDBTBPDBq-II:PCBBiF:[Ir(dmdpbq)₂(dpm)](0.7:0.3:0.1 40 nm)

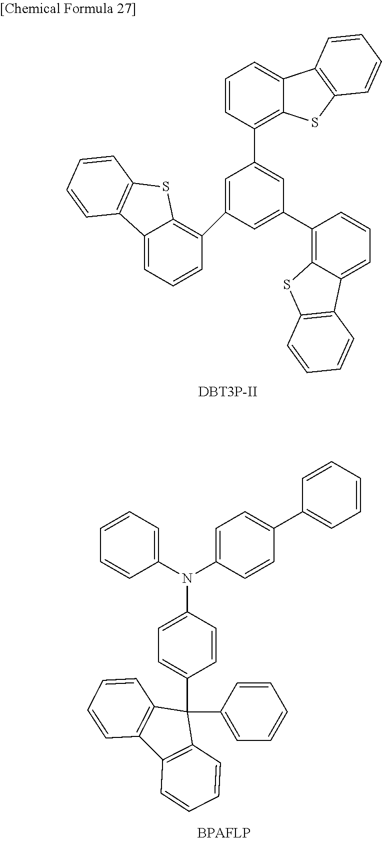 [SMILES: c1ccc2c(c1)sc1c(-c3cc(-c4cccc5c4sc4ccccc45)cc(-c4cccc5c4sc4ccccc45)c3)cccc12 ; "DBT3P-II" ; c1ccc(-c2ccc(N(c3ccccc3)c3ccc(C4(c5ccccc5)c5ccccc5-c5ccccc54)cc3)cc2)cc1]

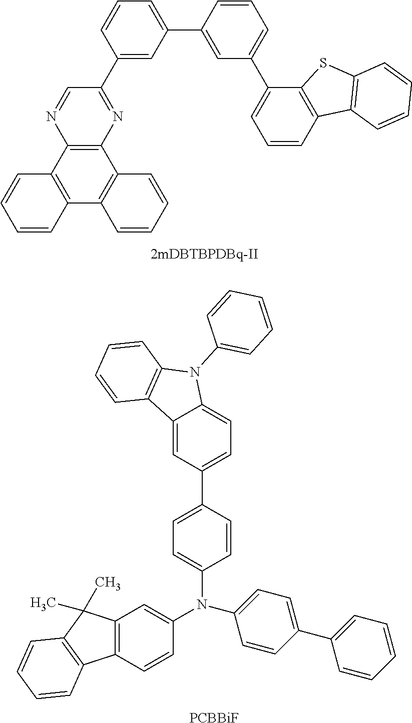

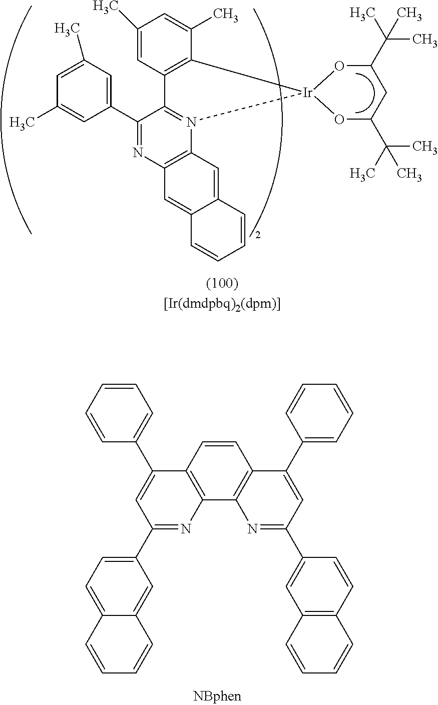

### Fabrication of Light-Emitting Device 1

The light-emitting device 1 described in this example has a structure in which a first electrode 801 is formed over a substrate 800; a hole-injection layer 811, a hole-transport layer 812, a light-emitting layer 813, an electron-transport layer 814, and an electron-injection layer 815 are stacked in this order over the first electrode 801; and a second electrode 803 is stacked over the electron-injection layer 815, as illustrated in FIG. 9A.

First, the first electrode 801 was formed over the substrate 800. The electrode area was set to 4 mm2 (2 mm×2 mm). A glass substrate was used as the substrate 800. The first electrode 801 was formed to a thickness of 110 nm using indium tin oxide containing silicon oxide (ITSO) by a sputtering method. In this example, the first electrode 801 functions as an anode.

As pretreatment, a surface of the substrate was washed with water, baking was performed at 200° C. for one hour, and then UV ozone treatment was performed for 370 seconds. After that, the substrate was transferred into a vacuum evaporation apparatus in which the pressure was reduced to approximately 10⁻⁴ Pa, vacuum baking at 170° C. for 30 minutes was performed in a heating chamber in the vacuum evaporation apparatus, and then the substrate was cooled down for approximately 30 minutes.

Next, the hole-injection layer 811 was formed over the first electrode 801. For the formation of the hole-injection layer 811, the pressure in the vacuum evaporation apparatus was reduced to 10⁻⁴ Pa, and then 1,3,5-tri(dibenzothiophen-4-yl)benzene (abbreviation: DBT3P-II) and molybdenum oxide were co-evaporated such that DBT3P-II: molybdenum oxide=2:1 (weight ratio) and the thickness was 60 nm.

Then, the hole-transport layer 812 was formed over the hole-injection layer 811. The hole-transport layer 812 was formed to a thickness of 20 nm by evaporation of 4-phenyl-4′-(9-phenylfluoren-9-yl)triphenylamine (abbreviation: BPAFLP).

Next, the light-emitting layer 813 was formed over the hole-transport layer 812. Using 2-[3′-(dibenzothiophen-4-yl)diphenyl-3-yl]dibenzo[f,h]quinoxaline (abbreviation: 2mDBTBPDBq-II) as a host material, N-(1,1′-biphenyl-4-yl)-N-[4-(9-phenyl-9H-carbazol-3-yl)phenyl]-9,9-dimethyl-9H-fluoren-2-amine (abbreviation: PCBBiF) as an assist material, and [Ir(dmdpbq)₂(dpm)], which is the organometallic complex of one embodiment of the present invention, as a guest material (a phosphorescent material), co-evaporation was performed so that the weight ratio was 2mDBTBPDBq-II: PCBBiF: [Ir(dmdpbq)₂(dpm)]=0.7:0.3:0.1. The thickness was set to 40 nm.

Next, the electron-transport layer 814 was formed over the light-emitting layer 813. The electron-transport layer 814 was formed by sequential deposition by evaporation so that the thickness of 2mDBTBPDBq-II was 20 nm and the thickness of 2,9-bis(naphthalen-2-yl)-4,7-diphenyl-1,10-phenanthroline (abbreviation: NBphen) was 70 nm.

Then, the electron-injection layer 815 was formed over the electron-transport layer 814. The electron-injection layer 815 was formed to a thickness of 1 nm by evaporation of lithium fluoride (LiF).

Next, the second electrode 803 was formed over the electron-injection layer 815. The second electrode 803 was formed to a thickness of 200 nm by an evaporation method using aluminum. In this example, the second electrode 803 functions as a cathode.

Through the above steps, the light-emitting device in which an EL layer 802 was provided between the pair of electrodes over the substrate 800 were fabricated. The hole-injection layer 811, the hole-transport layer 812, the light-emitting layer 813, the electron-transport layer 814, and the electron-injection layer 815 described in the above steps are functional layers forming the EL layer in one embodiment of the present invention. Furthermore, in all the evaporation steps in the above fabrication method, an evaporation method by a resistance-heating method was used.

The light-emitting device fabricated as described above was sealed using a different substrate (not illustrated). At the time of the sealing using the different substrate (not illustrated), the different substrate (not illustrated) on which an adhesive that is solidified by ultraviolet light was applied was fixed onto the substrate 800 in a glove box containing a nitrogen atmosphere, and the substrates were bonded to each other such that the adhesive was attached to the periphery of the light-emitting device formed over the substrate 800. At the time of the sealing, the adhesive was irradiated with 365-nm ultraviolet light at 6 J/cm² to be solidified, and the adhesive was subjected to heat treatment at 80° C. for one hour to be stabilized.

### Operating Characteristics of Light-Emitting Device 1

The operating characteristics of the light-emitting device 1 were measured. Note that the measurement was carried out at room temperature (an atmosphere maintained at 25° C.).

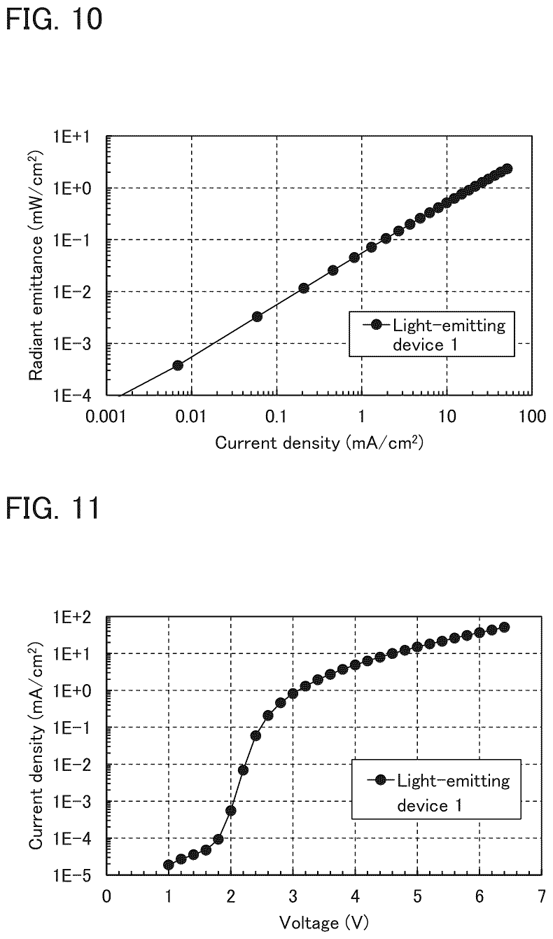

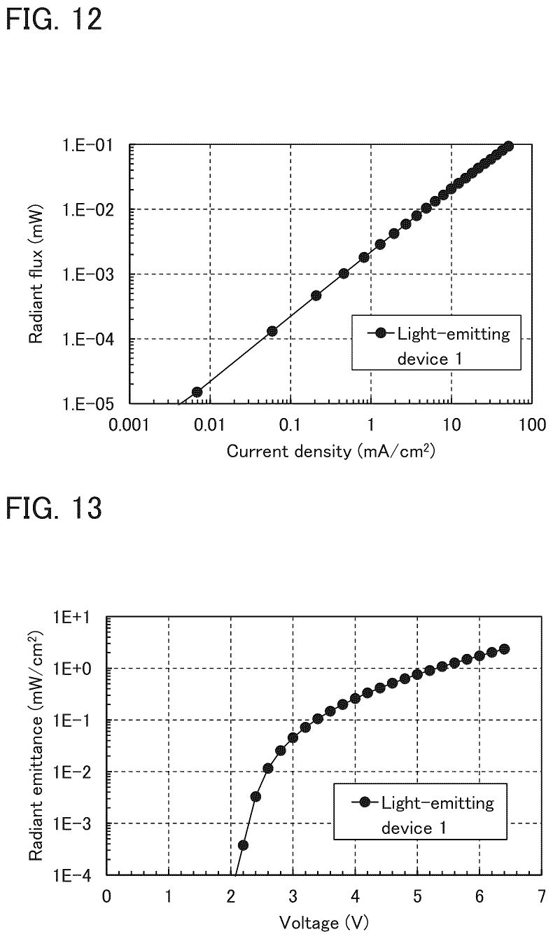

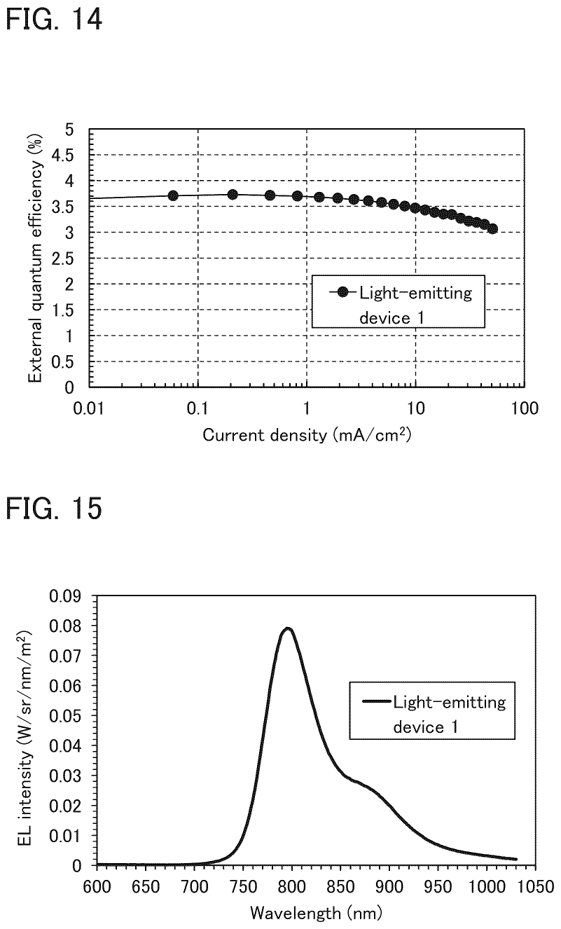

FIG. 10 shows the current density-radiant emittance characteristics of the light-emitting device 1. FIG. 11 shows the voltage-current density characteristics of the light-emitting device 1. FIG. 12 shows the current density-radiant flux characteristics of the light-emitting device 1. FIG. 13 shows the voltage-radiant emittance characteristics of the light-emitting device 1. FIG. 14 shows the current density-external quantum efficiency characteristics of the light-emitting device 1. Note that radiant emittance, radiant flux, and external quantum efficiency were calculated using radiance, assuming that the light-emitting device had Lambertian light-distribution characteristics.

Table 2 lists the initial values of main characteristics of the light-emitting device 1 at around 7.4 W/sr/m².

TABLE 2

| | | | Current | | | External |
|---|---|---|---|---|---|---|
| | | | density | | Radiant | quantum |
| | Voltage | Current | (mA/ | Radiance | flux | efficiency |
| | (V) | (mA) | cm²) | (W/sr/m²) | (mW) | (%) |
| Light- | 6.4 | 2.00 | 51 | 7.4 | 0.09 | 3.2 |
| emitting | | | | | | |
| device 1 | | | | | | |

The light-emitting device 1 was found to exhibit favorable characteristics, as shown in FIG. 10 to FIG. 14 and Table 2.

FIG. 15 shows an emission spectrum when current at a current density of 51 mA/cm² was supplied to the light-emitting device 1. The emission spectrum was measured with a near-infrared spectroradiometer (SR-NIR, manufactured by TOPCON TECHNOHOUSE CORPORATION). As shown in FIG. 15, the light-emitting device 1 exhibited an emission spectrum having a maximum peak at around 796 nm, which was derived from light emitted from [Ir(dmdpbq)₂(dpm)] contained in the light-emitting layer 813.

The half width of the emission spectrum was 67 nm. Energy obtained by conversion of the half width is approximately 0.13 eV, which is considerably narrow for light derived from an organometallic complex. The light-emitting device 1 efficiently emits light from 760 nm to 900 nm (or light from 780 nm to 880 nm) and is said to be highly effective as a light source for a sensor application and the like.

### Reliability Test on Light-Emitting Device 1

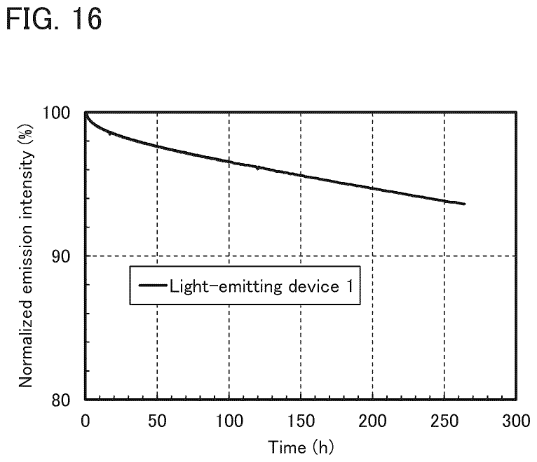

Next, a reliability test was performed on the light-emitting device 1. FIG. 16 shows the results of the reliability test. In FIG. 16, the vertical axis represents a normalized emission intensity (%) given that the initial emission intensity is 100%, and the horizontal axis represents driving time (h). In the reliability test, the light-emitting device 1 was driven at a current density of 75 mA/cm².

The results of the reliability test showed that light-emitting device 1 has high reliability. This can be regarded as the effect of using [Ir(dmdpbq)₂(dpm)] (Structural Formula (100)), which is the organometallic complex of one embodiment of the present invention, in the light-emitting layer of the light-emitting device 1.

### EXAMPLE 3

In this example, the results of fabricating a light-emitting device of one embodiment of the present invention will be described. A light-emitting device 2 fabricated in this example is an example of a light-emitting device in which a light-emitting layer contains a light-emitting organic compound and the maximum peak wavelength of light emitted from the light-emitting organic compound is greater than or equal to 760 nm and less than or equal to 900 nm. The results of fabricating the light-emitting device 2 and measuring its characteristics are described below.

In the light-emitting device 2, bis(dibenzo[a,i]naphtho[2,1-c]phenazine-10-yl-κC¹⁰,κN¹¹)(2,2,6,6-tetramethyl-3,5-heptanedionato-κ²O,O′)iridium(III) (abbreviation: [Ir(dbnphz)₂(dpm)]) was used as the light-emitting organic compound in the light-emitting layer. That is, the light-emitting device 2 is an example of a light-emitting device using an organometallic complex having a metal-carbon bond as the light-emitting organic compound. Note that a synthesis example of [Ir(dbnphz)₂(dpm)] will be described later in Reference Example.

Table 3 shows specific components of the light-emitting device 2 used in this example. Note that the light-emitting device 2 has the same structure as the light-emitting device 1 (FIG. 9A); thus, Example 2 can be referred to for the fabrication method. The chemical formulae of the materials used in this example are shown below.

TABLE 3

| | | | Hole- | Light- | | | Electron- | |
|---|---|---|---|---|---|---|---|---|
| | First | Hole-injection | transport | emitting | | | injection | Second |
| | electrode | layer | layer | layer | Electron-transport layer | | layer | electrode |
| | 801 | 811 | 812 | 813 | 814 | | 815 | 803 |
| Light- | ITSO | DBT3P-II:MoOx | PCBBiF | * | 2mDBTBPDBq-II | NBphen | LiF | Al |
| emitting | (70 nm) | (2:1 120 nm) | (20 nm) | | (20 nm) | (70 nm) | (1 nm) | (200 nm) |
| device 2 | | | | | | | | |

*2mDBTBPDBq-II:PCBBiF:[Ir(dbnphz)₂(dpm)](0.7:0.3:0.1 40 nm)

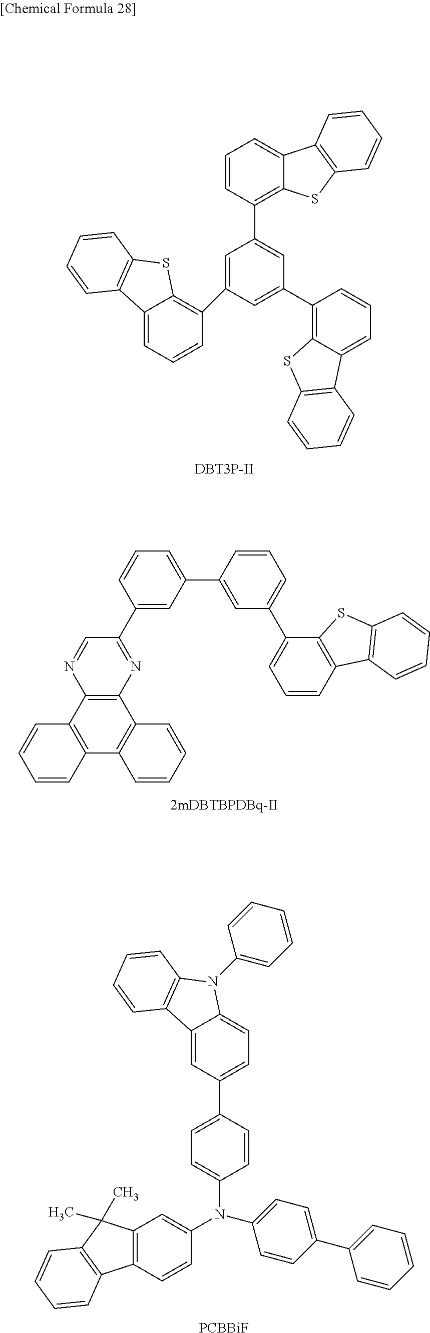

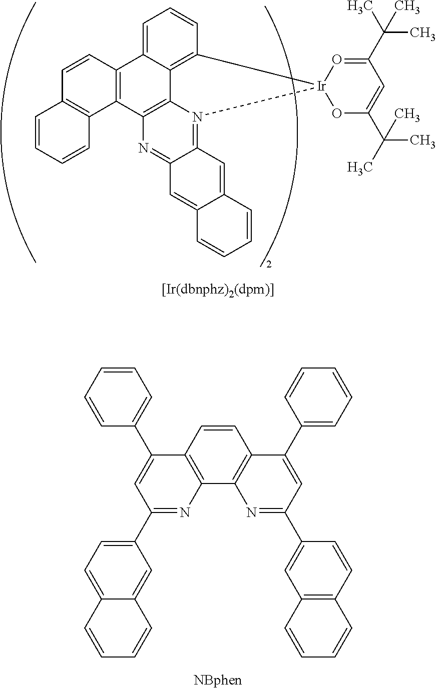

### Operating Characteristics of Light-Emitting Device 2

The operating characteristics of the light-emitting device 2 were measured. Note that the measurement was carried out at room temperature (an atmosphere maintained at 25° C.).

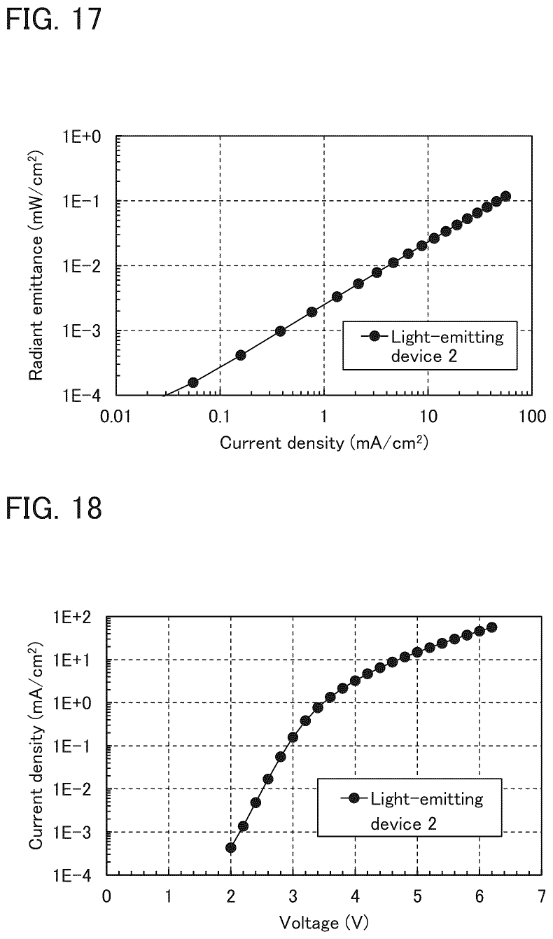

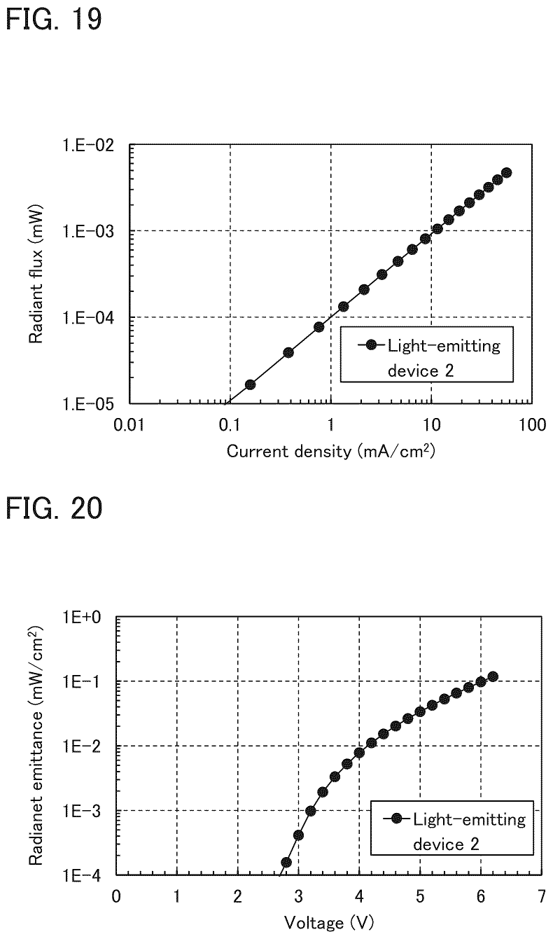

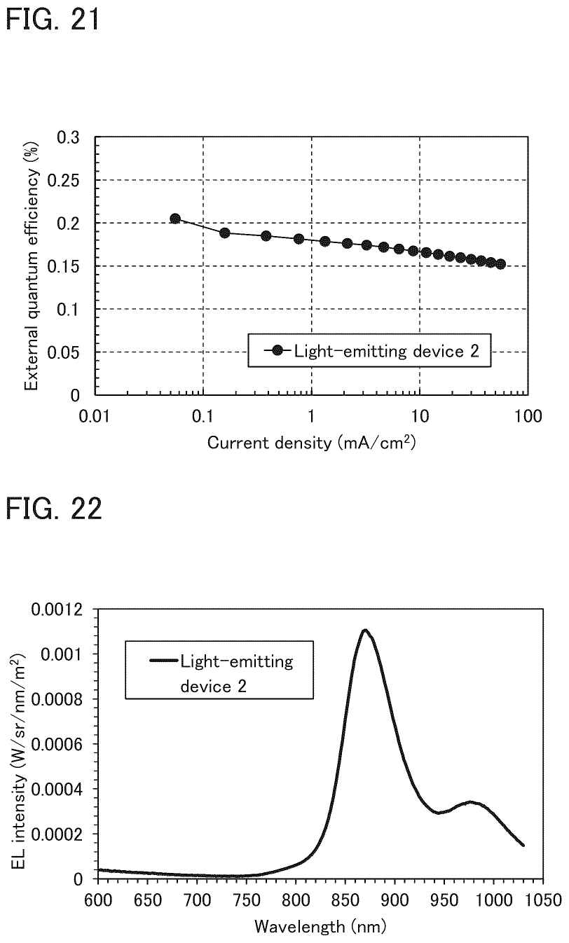

FIG. 17 shows the current density-radiant emittance characteristics of the light-emitting device 2. FIG. 18 shows the voltage-current density characteristics of the light-emitting device 2. FIG. 19 shows the current density-radiant flux characteristics of the light-emitting device 2. FIG. 20 shows the voltage-radiant emittance characteristics of the light-emitting device 2. FIG. 21 shows the current density-external quantum efficiency characteristics of the light-emitting device 2. Note that radiant emittance, radiant flux, and external quantum efficiency were calculated using radiance, assuming that the light-emitting device had Lambertian light-distribution characteristics.

Table 4 lists the initial values of main characteristics of the light-emitting device 2 at around 0.11 W/sr/m².

TABLE 4

| | | | Current | | | External |
|---|---|---|---|---|---|---|
| | | | density | | Radiant | quantum |
| | Voltage | Current | (mA/ | Radiance | flux | efficiency |
| | (V) | (mA) | cm²) | (W/sr/m²) | (mW) | (%) |
| Light- | 5.0 | 0.59 | 15 | 0.11 | 0.0013 | 0.16 |
| emitting | | | | | | |
| device 2 | | | | | | |

The light-emitting device 2 was found to exhibit favorable element characteristics, as shown in FIG. 17 to FIG. 21 and Table 4.

FIG. 22 shows an emission spectrum when current at a current density of 15 mA/cm² was supplied to the light-emitting device 2. The emission spectrum in FIG. 22 was measured with a near-infrared spectroradiometer (SR-NIR, manufactured by TOPCON TECHNOHOUSE CORPORATION). The light-emitting device 2 exhibits an emission spectrum having a maximum peak at around 870 nm, which was derived from light emitted from [Ir(dbnphz)₂(dpm)] contained in the light-emitting layer 813.

The half width of the emission spectrum was 63 nm. Energy obtained by conversion of the half width is approximately 0.10 eV, which is considerably narrow for light derived from an organometallic complex. The light-emitting device 2 efficiently emits light from 760 nm to 900 nm (or light from 780 nm to 880 nm) and is said to be highly effective as a light source for a sensor application and the like.

### Reliability Test on Light-Emitting Device 2

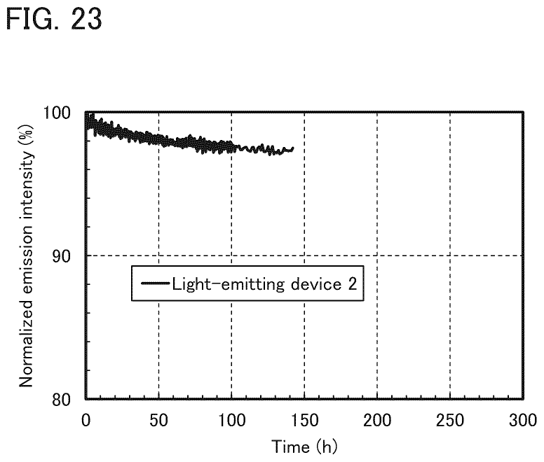

Next, a reliability test was performed on the light-emitting device 2. FIG. 23 shows the results of the reliability test. In FIG. 23, the vertical axis represents a normalized emission intensity (%) given that the initial emission intensity is 100%, and the horizontal axis represents driving time (h) of the element. In the reliability test, the light-emitting device 2 was driven at a current density of 75 mA/cm².

The results of the reliability test showed that the light-emitting device 2 has high reliability.

### EXAMPLE 4

In this example, the results of fabricating a light-emitting device of one embodiment of the present invention will be described. Specifically, the description is made on a structure, a fabrication method, and characteristics of a light-emitting device 3 using [Ir(dmdpbq)₂(dpm)], which is described in Example 1, in a light-emitting layer.

The light-emitting device 3 fabricated in this example is an example of a light-emitting device in which the light-emitting layer contains a light-emitting organic compound and the maximum peak wavelength of light emitted from the light-emitting organic compound is greater than or equal to 760 nm and less than or equal to 900 nm. Moreover, the light-emitting device 3 is an example of a light-emitting device using an organometallic complex having a metal-carbon bond as the light-emitting organic compound.

FIG. 9B illustrates a structure of the light-emitting device 3 used in this example. The light-emitting device 3 used in this example is a light-emitting device having a tandem structure in which two EL layers (an EL layer 802a and an EL layer 802b) are provided between a pair of electrodes (the first electrode 801 and the second electrode 803) and a charge-generation layer 816 is provided between the two EL layers.

Table 5 shows specific components of the light-emitting device 3 used in this example. The chemical formula of the material used in this example is shown below.

TABLE 5

| | First electrode | | → | | | | | |
|---|---|---|---|---|---|---|---|---|
| | 801 | | | | | | | |
| Light- | APC | ITSO | | | | | | |
| emitting | (100 nm) | (10 nm) | | | | | | |
| device 3 | | | | | | | | |
| → | | Hole- | Light- | | | Electron- | Charge- | → |
| | Hole-injection | transport | emitting | | | injection | generation | |
| | layer | layer | layer | Electron-transport layer | | layer | layer | |
| | 811a | 812a | 813a | 814a | | 815a | 816 | |
| | DBT3P-II: | PCBBiF | * | 2mDBTBPDBq-II | NBphen | Li₂O | CuPc | |
| | MoOx | (30 nm) | | (20 nm) | (90 nm) | (0.1 nm) | (2 nm) | |
| | (2:1 10 nm) | | | | | | | |
| → | | Hole- | Light- | | | Electron | → | |
| | Hole-injection | transport | emitting | | | injection | | |
| | layer | layer | layer | Electron-transport layer | | layer | | |
| | 811b | 812b | 813b | 814b | | 815b | | |
| | DBT3P-II: | PCBBiF | * | 2mDBTBPDBq-II | NBphen | LiF | | |
| | MoOx | (60 nm) | | (20 nm) | (65 nm) | (1 nm) | | |
| | (2:1 10 nm) | | | | | | | |
| → | Second | Buffer | | | | | | |
| | electrode | layer | | | | | | |
| | 803 | 804 | | | | | | |
| | Ag:Mg | DBT3P-II | | | | | | |
| | (10:1 | (110 nm) | | | | | | |
| | 20 nm) | | | | | | | |

*2mDBTBPDBq-II:PCBBiF:[Ir(dmdpbq)₂(dpm)](0.7:0.3:0.1 40 nm)

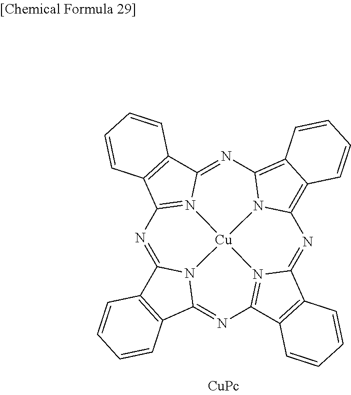

### Fabrication of Light-Emitting Device 3

The light-emitting device 3 described in this example has a structure in which the first electrode 801 is formed over the substrate 800; the EL layer 802a (a hole-injection layer 811a, a hole-transport layer 812a, a light-emitting layer 813a, an electron-transport layer 814a, and an electron-injection layer 815a), the charge-generation layer 816, and the EL layer 802b (a hole-injection layer 811b, a hole-transport layer 812b, a light-emitting layer 813b, an electron-transport layer 814b, and an electron-injection layer 815b) are stacked in this order over the first electrode 801; and the second electrode 803 is stacked over the EL layer 802b, as illustrated in FIG. 9B.

First, the first electrode 801 was formed over the substrate 800. The electrode area was set to 4 mm² (2 mm×2 mm). A glass substrate was used as the substrate 800. The first electrode 801 was formed in such a manner that an alloy film of silver (Ag), palladium (Pd), and copper (Cu) (Ag—Pd—Cu (APC)) was formed to a thickness of 100 nm by a sputtering method, and an ITSO film was formed to a thickness of 10 nm by a sputtering method. In this example, the first electrode 801 functions as an anode.

As pretreatment, a surface of the substrate was washed with water, baking was performed at 200° C. for one hour, and then UV ozone treatment was performed for 370 seconds. After that, the substrate was transferred into a vacuum evaporation apparatus in which the pressure was reduced to approximately 10⁻⁴ Pa, vacuum baking at 170° C. for 30 minutes was performed in a heating chamber in the vacuum evaporation apparatus, and then the substrate was cooled down for approximately 30 minutes.

Next, the hole-injection layer 811a was formed over the first electrode 801. For the formation of the hole-injection layer 811a, the pressure in the vacuum evaporation apparatus was reduced to 10⁻⁴ Pa, and then DBT3P-II and molybdenum oxide were co-evaporated such that DBT3P-II: molybdenum oxide=2:1 (weight ratio) and the thickness was 10 nm.

Then, the hole-transport layer 812a was formed over the hole-injection layer 811a. The hole-transport layer 812a was formed to a thickness of 30 nm by evaporation of PCBBIF.

Next, the light-emitting layer 813a was formed over the hole-transport layer 812a. Using 2mDBTBPDBq-II as a host material, PCBBiF as an assist material, and [Ir(dmdpbq)₂(dpm)], which is the organometallic complex of one embodiment of the present invention, as a guest material (a phosphorescent material), co-evaporation was performed so that the weight ratio was 2mDBTBPDBq-II: PCBBiF: [Ir(dmdpbq)₂(dpm)]=0.7:0.3:0.1. The thickness was set to 40 nm.

Next, the electron-transport layer 814a was formed over the light-emitting layer 813a. The electron-transport layer 814a was formed by sequential deposition by evaporation so that the thickness of 2mDBTBPDBq-II was 20 nm and the thickness of NBphen was 90 nm.

Then, the electron-injection layer 815a was formed over the electron-transport layer 814a. The electron-injection layer 815a was formed to a thickness of 0.1 nm by evaporation of lithium oxide (Li₂O).

Then, the charge-generation layer 816 was formed over the electron-injection layer 815a. The charge-generation layer 816 was formed to a thickness of 2 nm by evaporation of copper(II) phthalocyanine (CuPc).

Next, the hole-injection layer 811b was formed over the charge-generation layer 816. The hole-injection layer 811b was formed by co-evaporation of DBT3P-II and molybdenum oxide such that DBT3P-II: molybdenum oxide=2:1 (weight ratio) and the thickness was 10 nm.

Then, the hole-transport layer 812b was formed over the hole-injection layer 811b. The hole-transport layer 812b was formed to a thickness of 60 nm by evaporation of PCBBiF.

Next, the light-emitting layer 813b was formed over the hole-transport layer 812b. Using 2mDBTBPDBq-II as a host material, PCBBiF as an assist material, and [Ir(dmdpbq)₂(dpm)], which is the organometallic complex of one embodiment of the present invention, as a guest material (a phosphorescent material), co-evaporation was performed so that the weight ratio was 2mDBTBPDBq-II:PCBBiF:[Ir(dmdpbq)₂(dpm)]=0.7:0.3:0.1. The thickness was set to 40 nm.

Next, the electron-transport layer 814b was formed over the light-emitting layer 813b. The electron-transport layer 814b was formed by sequential deposition by evaporation so that the thickness of 2mDBTBPDBq-II was 20 nm and the thickness of NBphen was 65 nm.

Then, the electron-injection layer 815b was formed over the electron-transport layer 814b. The electron-injection layer 815b was formed to a thickness of 1 nm by evaporation of lithium fluoride (LiF).

After that, the second electrode 803 was formed over the electron-injection layer 815b. The second electrode 803 was formed by co-evaporation of silver (Ag) and magnesium (Mg) such that Ag:Mg=10:1 (volume ratio) and the thickness was 20 nm. In this example, the second electrode 803 functions as a cathode.

Next, a buffer layer 804 was formed over the second electrode 803. The buffer layer 804 was formed to a thickness of 110 nm by evaporation of DBT3P-II.

Through the above steps, the light-emitting device 3 was formed over the substrate 800. In all the evaporation steps in the above fabrication method, an evaporation method by a resistance-heating method was used.

The light-emitting device 3 was sealed using another substrate (not illustrated). The sealing method is the same as that for the light-emitting device 1; hence, Example 2 can be referred to for the sealing method.

Note that a microcavity structure is applied to the light-emitting device 3. The light-emitting device 3 was fabricated so that the optical distance between the pair of reflective electrodes (the APC film and the Ag:Mg film) was approximately one-wave of the maximum peak wavelength of light emitted from the guest material.

### Operating Characteristics of Light-Emitting Device 3

The operating characteristics of the light-emitting device 3 were measured. Note that the measurement was carried out at room temperature (an atmosphere maintained at 25° C.).

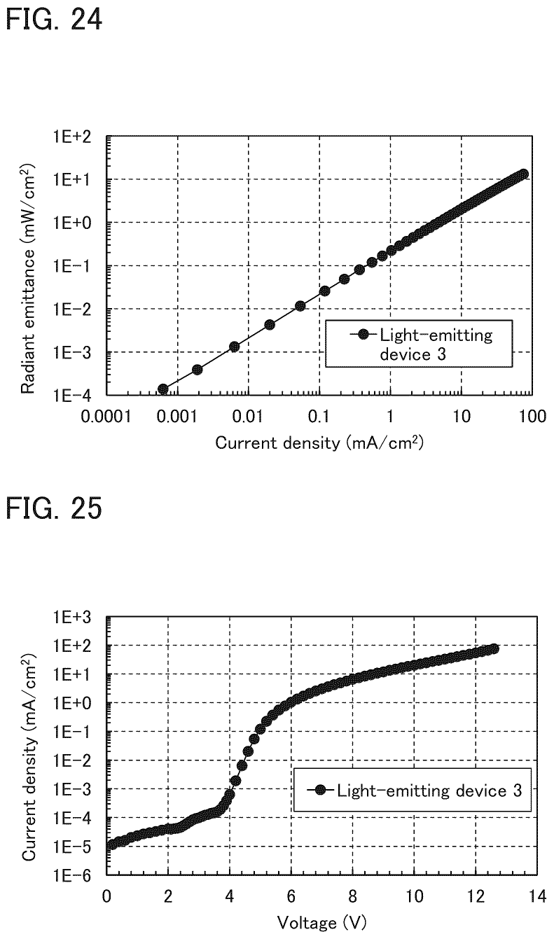

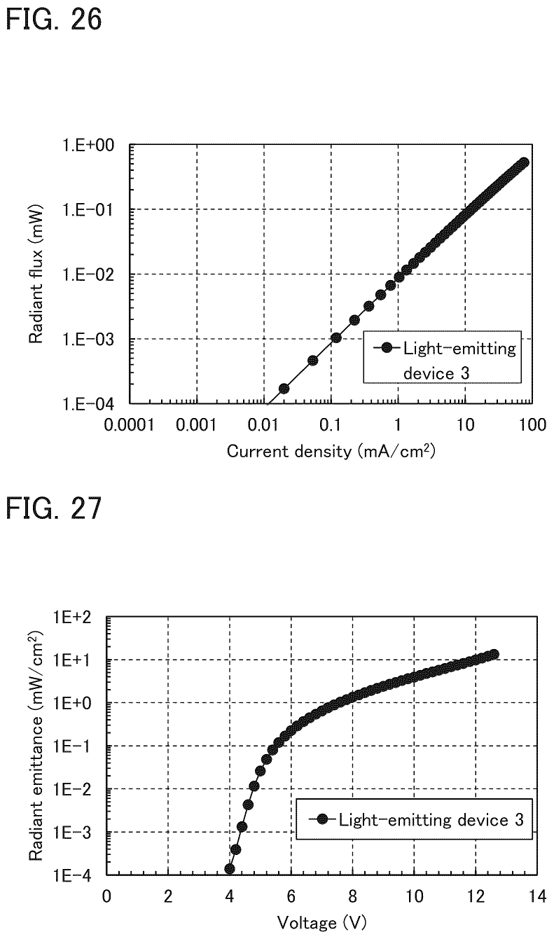

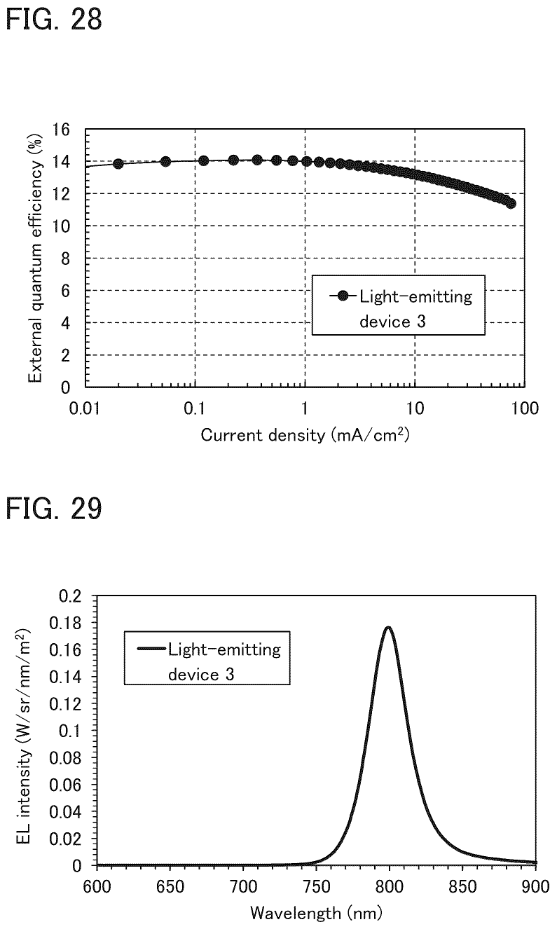

FIG. 24 shows the current density-radiant emittance characteristics of the light-emitting device 3. FIG. 25 shows the voltage-current density characteristics of the light-emitting device 3. FIG. 26 shows the current density-radiant flux characteristics of the light-emitting device 3. FIG. 27 shows the voltage-radiant emittance characteristics of the light-emitting device 3. FIG. 28 shows the current density-external quantum efficiency characteristics of the light-emitting device 3. Note that radiant emittance, radiant flux, and external quantum efficiency were calculated using radiance, assuming that the light-emitting device had Lambertian light-distribution characteristics.

Table 6 lists the initial values of main characteristics of the light-emitting device 3 at around 6.8 W/sr/m².

TABLE 6

| | | | Current | | | External |
|---|---|---|---|---|---|---|
| | | | density | | Radiant | quantum |
| | Voltage | Current | (mA/ | Radiance | flux | efficiency |
| | (V) | (mA) | cm²) | (W/sr/m²) | (mW) | (%) |
| Light- | 8.8 | 0.42 | 11 | 6.8 | 0.09 | 13 |
| emitting | | | | | | |
| device 3 | | | | | | |

The light-emitting device 3 was found to exhibit favorable characteristics, as shown in FIG. 24 to FIG. 28 and Table 6. Since the light-emitting device 3 has a tandem structure, the results with a high peak value of the EL intensity and high quantum efficiency can be obtained.

FIG. 29 shows an emission spectrum when current at a current density of 10 mA/cm² was supplied to the light-emitting device 3. The emission spectrum was measured with a near-infrared spectroradiometer (SR-NIR, manufactured by TOPCON TECHNOHOUSE CORPORATION). As shown in FIG. 29, the light-emitting device 3 exhibited an emission spectrum having a maximum peak at around 799 nm, which was derived from light emitted from [Ir(dmdpbq)₂(dpm)] contained in the light-emitting layer 813a and the light-emitting layer 813b.

Employing the microcavity structure narrowed the emission spectrum, and the half width was 32 nm. The light-emitting device 3 efficiently emits light from 760 nm to 900 nm (or light from 780 nm to 880 nm) and is said to be highly effective as a light source for a sensor application and the like.

### Reliability Test on Light-Emitting Device 3

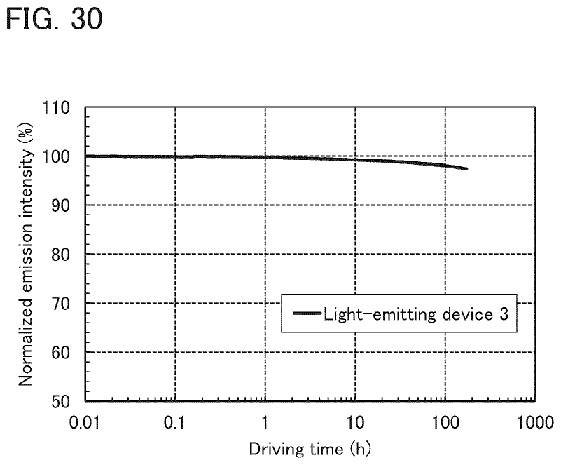

Next, a reliability test was performed on the light-emitting device 3. FIG. 30 shows the results of the reliability test. In FIG. 30, the vertical axis represents a normalized emission intensity (%) given that the initial emission intensity is 100%, and the horizontal axis represents driving time (h). In the reliability test, the light-emitting device 3 was driven at a current density of 75 mA/cm².

The results of the reliability test showed that the light-emitting device 3 has high reliability. This can be regarded as the effect of using [Ir(dmdpbq)₂(dpm)] (Structural Formula (100)), which is the organometallic complex of one embodiment of the present invention, in the light-emitting layer of the light-emitting device 3.

### Viewing Angle Characteristics of Light-Emitting Device 3

Next, the viewing angle characteristics of the EL spectra of the light-emitting device 3 were examined.

First, the EL spectra in the front direction and the EL spectra in oblique directions of the light-emitting element were measured. Specifically, given that the direction perpendicular to a light-emitting surface of the light-emitting device 3 was 0°, emission spectra were measured at a total of 17 points at every 10° from −80° to 80°. For the measurement, a multi-channel spectrometer (Hamamatsu Photonics K.K., PMA-12) was used. From the measurement results, the EL spectra and the photon intensity ratio of the light-emitting element at each angle were obtained.

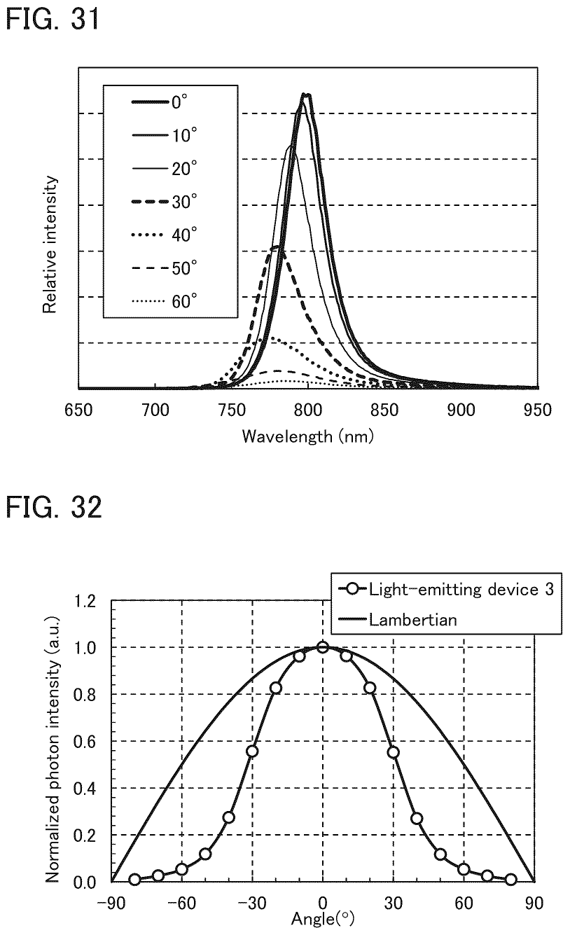

FIG. 31 shows the EL spectra of the light-emitting device 3 from 0° to 60°.

FIG. 32 shows the photon intensity (Normalized photon intensity) of the light-emitting device 3 at each angle relative to the photon intensity at the front. FIG. 32 also shows Lambertian characteristics.

As shown in FIG. 31 and FIG. 32, the light-emitting device 3 was found to have large viewing angle dependence and emit intense light in the front direction. This is because employing the microcavity structure reduces light emission in the oblique directions while intensifying light emission in the front direction. Such viewing angle characteristics with intense light emission in the front direction are suitable for a light source for a sensor such as a vein sensor.

### Reference Example

A method for synthesizing bis(dibenzo[a,i]naphtho[2, 1-c]phenazine-10-yl-κC¹⁰,κN¹¹)(2,2,6,6-tetramethyl-3,5-heptanedionato-κ²O,O′)iridium(III) (abbreviation: [Ir(dbnphz)2(dpm)]), which is used in Example 3 described above, will be specifically described. The structure of [Ir(dbnphz)2(dpm)] is shown below.

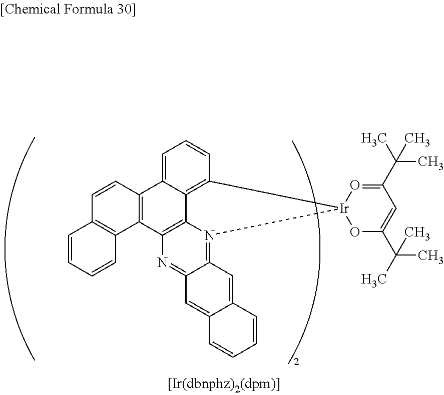

Step 1: Synthesis of dibenzo[a,i]naphtho[2,1-c]phenazine (Abbreviation: Hdbnphz)

First, 1.0 g (4.0 mmol) of chrysene-5,6-dione, 0.67 g (4.3 mmol) of 2,3-diaminonaphthalene, and 20 mL of ethanol were put into a reaction container, and heated and refluxed for 5 hours. After a predetermined time elapsed, the obtained mixture was subjected to suction filtration, and the solid was washed with ethanol. This solid was dissolved in heated toluene, followed by suction filtration through a filter medium in which Celite, alumina, and Celite are stacked in this order. The obtained filtrate was concentrated and recrystallization was performed with a mixed solvent of toluene and ethanol, whereby the target substance was obtained (1.1 g, percent yield: 74%). The synthesis scheme of Step 1 is shown in (b-1).

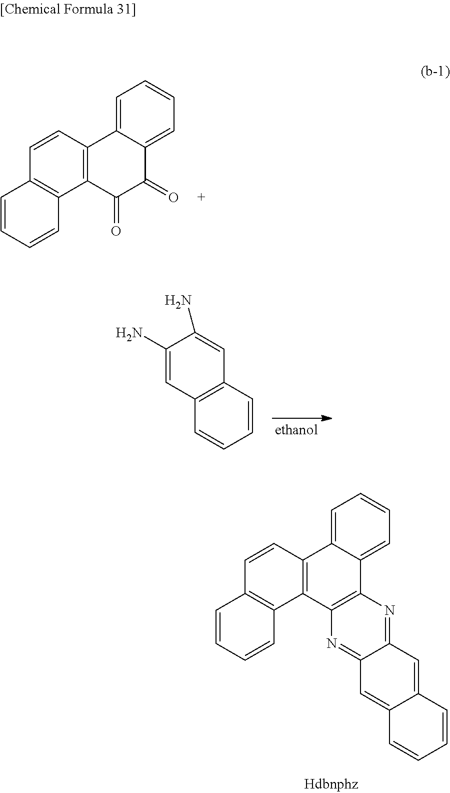 [SMILES: O=C1C(=O)c2c(ccc3ccccc23)-c2ccccc21 ; Nc1cc2ccccc2cc1N ; c1ccc2cc3nc4c(nc3cc2c1)c1ccccc1c1ccc2ccccc2c14]

### Step 2: Synthesis of [Ir(dbnphz)₂(dpm)]

Next, 1.1 g (2.9 mmol) of Hdbnphz obtained in Step 1, 0.39 g (1.3 mmol) of iridium chloride hydrate, and 30 mL of dimethylformamide (DMF) were added to a reaction container, the air in the container was replaced with nitrogen, and the mixture was heated and stirred at 160° C. for 7.5 hours. After a predetermined time elapsed, 0.55 g (5.2 mmol) of sodium carbonate and 0.72 g (3.9 mmol) of dipivaloylmethane were added, and the mixture was heated and stirred at 140° C. for 14 hours. Then, this mixture was suction-filtered, and the obtained solid was washed with water and ethanol.

Subsequently, this solid was purified by silica gel column chromatography using dichloromethane as a developing solvent, and the obtained fraction was concentrated to give a solid. This solid was washed with heated toluene to give 133 mg of the target substance.

Then, the obtained filtrate was concentrated and purified by silica gel column chromatography using toluene as a developing solvent. Next, the solid obtained by concentrating the obtained fraction was recrystallized using a mixed solvent of toluene and ethanol, whereby the target substance (80 mg) was obtained (total yield: 213 mg, percent yield: 14%). The synthesis scheme of Step 2 is shown in Formula (b-2).

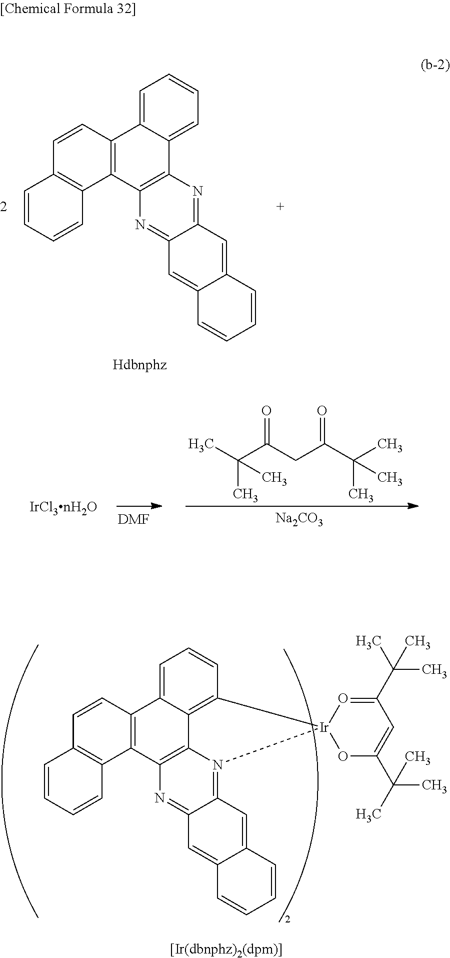

Protons (¹H) of the black solid obtained in Step 2 were measured by nuclear magnetic resonance spectroscopy (NMR). The obtained values are shown below. The measurement results show that [Ir(dbnphz)₂(dpm)]) was obtained.

¹H-NMR δ (CDCl₃): 0.52 (s, 18H), 5.04 (s, 1H), 6.80 (d, 2H), 6.97 (t, 2H), 7.48 (d, 2H), 7.59 (t, 2H), 7.74 (t, 2H), 7.88 (d, 2H), 7.98 (t, 2H), 8.06 (d, 2H), 8.10 (d, 2H), 8.26 (d, 4H), 8.64 (d, 2H), 9.13 (s, 2H), 9.19 (s, 2H), 11.21 (d, 2H).

### REFERENCE NUMERALS

101: first electrode, 102: second electrode, 103: EL layer, 103a: EL layer, 103b: EL layer, 104: charge-generation layer, 111: hole-injection layer, 112: hole-transport layer, 113: light-emitting layer, 114: electron-transport layer, 115: electron-injection layer, 301: first substrate, 302: pixel portion, 303: circuit portion, 304a: circuit portion, 304b: circuit portion, 305: sealant, 306: second substrate, 307: wiring, 308: FPC, 309: transistor, 310: transistor, 311: transistor, 312: transistor, 313: first electrode, 314: insulating layer, 315: EL layer, 316: second electrode, 317: organic EL device, 318: space, 401: first electrode, 402: EL layer, 403: second electrode, 405: insulating layer, 406: conductive layer, 407: adhesive layer, 416: conductive layer, 420: substrate, 422: adhesive layer, 423: barrier layer, 424: insulating layer, 450: organic EL device, 490a: substrate, 490b: substrate, 490c: barrier layer, 800: substrate, 801: first electrode, 802: EL layer, 802a: EL layer, 802b: EL layer, 803: second electrode, 804: buffer layer, 811: hole-injection layer, 811a: hole-injection layer, 811b: hole-injection layer, 812: hole-transport layer, 812a: hole-transport layer, 812b: hole-transport layer, 813: light-emitting layer, 813a: light-emitting layer, 813b: light-emitting layer, 814: electron-transport layer, 814a: electron-transport layer, 814b: electron-transport layer, 815: electron-injection layer, 815a: electron-injection layer, 815b: electron-injection layer, 816: charge-generation layer, 911: housing, 912: light source, 913: sensing stage, 914: imaging device, 915: light-emitting portion, 916: light-emitting portion, 917: light-emitting portion, 921: housing, 922: operation button, 923: sensing portion, 924: light source, 925: imaging device, 931: housing, 932: operation panel, 933: transport mechanism, 934: monitor, 935: sensing unit, 936: test specimen, 937: imaging device, 938: light source, 981: housing, 982: display portion, 983: operation button, 984: external connection port, 985: speaker, 986: microphone, 987: camera, 988: camera

1. An organometallic complex represented by General Formula (G1) 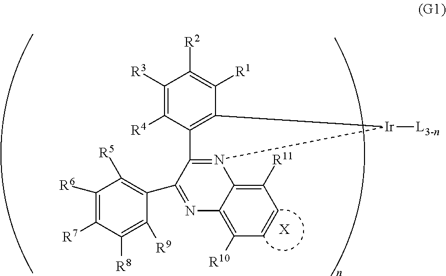
wherein each of R¹ to R¹¹ independently represents hydrogen or an alkyl group having 1 to 6 carbon atoms,
wherein at least two of R¹ to R⁴ represent an alkyl group having 1 to 6 carbon atoms,
wherein at least two of R⁵ to R⁹ represent an alkyl group having 1 to 6 carbon atoms,
wherein X represents a substituted or unsubstituted benzene ring or naphthalene ring,
wherein n is 2 or 3,
wherein L represents a monoanionic ligand,
wherein a maximum peak wavelength of light emitted from the organometallic complex is greater than or equal to 760 nm and less than or equal to 900 nm, and
wherein an energy of a half width of a maximum peak of the light emitted from the organometallic complex is less than or equal to 0.13 eV.

2. The organometallic complex according to claim 1,
wherein the energy of the half width of the maximum peak of the light emitted from the organometallic complex is more than or equal to 0.10 eV and less than or equal to 0.13 eV.

3. The organometallic complex according to claim 1,
wherein X represents a substituted or unsubstituted benzene ring.

4. The organometallic complex according to claim 1,
wherein each of R¹, R³, R⁶, and R⁸ represents a methyl group.

5. The organometallic complex according to claim 1,
wherein the organometallic complex is represented by General Formula (G2).

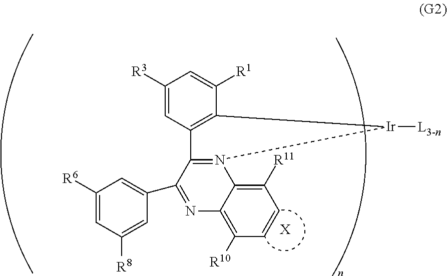

6. The organometallic complex according to claim 1,
wherein the maximum peak wavelength of light emitted from the organometallic complex is greater than or equal to 780 nm and less than or equal to 880 nm.

7. A light-emitting device comprising:
a light-emitting layer between a pair of electrodes,
wherein the light-emitting layer comprises the organometallic complex according to claim

1.

8. An organometallic complex represented by General Formula (G1), 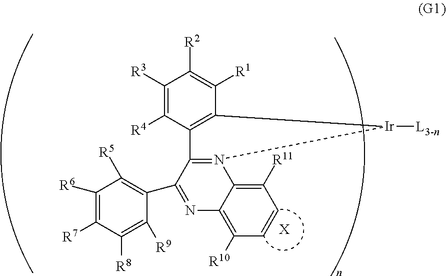
wherein each of R¹ to R¹¹ independently represents hydrogen or an alkyl group having 1 to 6 carbon atoms,
wherein at least two of R¹ to R⁴ represent an alkyl group having 1 to 6 carbon atoms,
wherein at least two of R⁵ to R⁹ represent an alkyl group having 1 to 6 carbon atoms,
wherein X represents a substituted or unsubstituted benzene ring or naphthalene ring,
wherein n is 2 or 3,
wherein L represents a monoanionic ligand,
wherein a maximum peak wavelength of light emitted from the organometallic complex is greater than or equal to 760 nm and less than or equal to 900 nm, and
wherein a half width of a maximum peak of the light emitted from the organometallic complex is narrower than or equal to 67 nm.

9. The organometallic complex according to claim 8,
wherein the half width of the maximum peak of the light emitted from the organometallic complex is wider than or equal to 32 nm and narrower than or equal to 67 nm.

10. The organometallic complex according to claim 9,
wherein the half width of the maximum peak of the light emitted from the organometallic complex is wider than or equal to 63 nm and narrower than or equal to 67 nm.

11. The organometallic complex according to claim 8,
wherein X represents a substituted or unsubstituted benzene ring.

12. The organometallic complex according to claim 8,
wherein each of R¹, R³, R⁶, and R⁸ represents a methyl group.

13. The organometallic complex according to claim 8,
wherein the organometallic complex is represented by General Formula (G2).

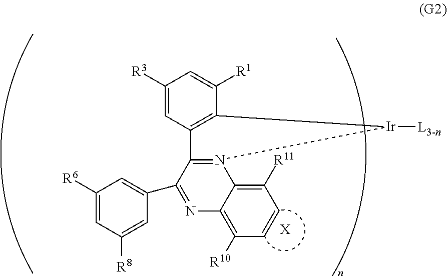

14. The organometallic complex according to claim 8,
wherein the maximum peak wavelength of the light emitted from the organometallic complex is greater than or equal to 780 nm and less than or equal to 880 nm.

15. A light-emitting device comprising:
a light-emitting layer between a pair of electrodes,
wherein the light-emitting layer comprises the organometallic complex according to claim 8.

16. An organometallic complex comprising a dibenzo[a,i]naphtho[2,1-c]phenazine skeleton as a ligand,
wherein a maximum peak wavelength of light emitted from the organometallic complex is greater than or equal to 760 nm and less than or equal to 900 nm, and
wherein a half width of a maximum peak of the light emitted from the organometallic complex is narrower than or equal to 67 nm.

17. The organometallic complex according to claim 16,
wherein the organometallic complex is represented by Chemical Structure (112).

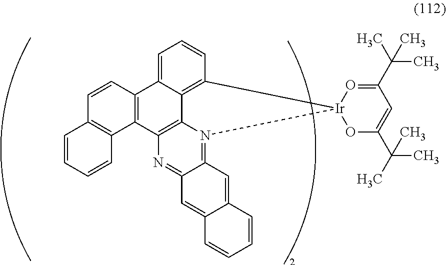

18. A light-emitting device comprising:
a light-emitting layer between a pair of electrodes,
wherein the light-emitting layer comprises the organometallic complex according to claim 16.
